(19) United States
(12) Patent Application Publication
(10) Pub. No.: US 2025/0161360 A1
(43) Pub. Date: May 22, 2025

(54) COMPOSITIONS AND METHODS OF SYNTHETIC CTLA-4 TAILS FOR REPROGRAMMING OF CAR-T CELLS AND ENHANCEMENT OF ANTI-TUMOR EFFICACY

(71) Applicant: Yale University, New Haven, CT (US)

(72) Inventors: Sidi Chen, Milford, CT (US); Xiaoyu Zhou, Hamden, CT (US)

(21) Appl. No.: 18/864,862

(22) PCT Filed: May 10, 2023
(86) PCT No.: PCT/US2023/066838

§ 371 Date: Nov. 11, 2024

Related U.S. Application Data
(60) Provisional application No. 63/340,296, filed on May 10, 2022.

(51) Int. Cl. A61K 35/17; A61K 40/11; A61K 40/31; A61K 40/42; A61P 35/02; C07K 14/705; C07K 16/28; C12N 15/86

(52) U.S. Cl. CPC A61K 35/17; A61K 40/11; A61K 40/31; A61K 40/4211; A61K 40/4212; A61P 35/02; C07K 14/70521; C07K 16/2803; C12N 15/86; A61K 2239/13; A61K 2239/22; C12N 2740/15043

(57) ABSTRACT

Compositions and methods for improved ACT are provided. Compositions of CAR-T cells including CAR modified by fusion with one or more copies of a polypeptide including from 20 to 44 amino acids from the transmembrane and/or cytosolic domain of CTLA-4 (CT) are described. CAR-CT-T cells have reduced trogocytosis, reduced T-cell fratricide, enhanced tumor antigen presentation and overall enhanced anti-tumor efficacy as compared with CAR-T cells lacking the CT domain(s). In preferred embodiments, CAR-TC fusion peptides include a CT domain having 2 copies of the YVKM motif. The compositions and methods provide enhanced CAR-T cell therapy for cancer, auto-immune disease as well as other disease and disorders. Also disclosed are genetically modified cells and pharmaceutical compositions and methods of use thereof for treating subjects having diseases or disorders.

### CROSS-REFERENCE TO RELATED APPLICATIONS

This application claims the benefit of and priority to U.S. Provisional Application No. 63/340,296 filed May 10, 2022, which is hereby incorporated by reference in its entirety.

### STATEMENT REGARDING FEDERALLY SPONSORED RESEARCH

This invention was made with government support under CA238295 and CA231112 awarded by the National Institutes of Health and under W81XWH-20-1-0072 awarded by the US Department of Defense (DoD). The Government has certain rights in the invention.

### REFERENCE TO SEQUENCE LISTING

The Sequence Listing submitted May 10, 2023 as a text file named “YU_8403_ST26,” created on May 10, 2023, and having a size of 129,906 bytes is hereby incorporated by reference pursuant to 37 C.F.R. § 1.52(e)(5).

### FIELD OF THE INVENTION

The invention is generally related to the fields of immunotherapy, and more particularly to improved chimeric antigen receptor (CAR) T cells with enhanced anti-tumor efficacy, and methods of making and using thereof.

### BACKGROUND OF THE INVENTION

Adoptive immunotherapy, in which T cells that are specific for tumor-associated antigens are expanded to generate large numbers of cells and transferred into tumor-bearing hosts, is a promising strategy to treat cancer.

Cellular immunotherapy involves the administration of “living drugs”: genetically modified immune cells that can proliferate, adapt to their environment, engage surrounding cells, and elicit dynamic responses that directly or indirectly target tumor cells for destruction (Hayes, Ir J Med Sci 190, 41-57, doi:10.1007/s11845-020-02264-w (2021).

Adoptive cell transfer (ACT) is one type of cellular immunotherapy which involves the transfer of cells that directly target tumor cells in the patient (Laskowski & Rezvani, J Exp Med 217, doi:10.1084/jem.20200377 (2020)). One notable ACT approach is chimeric antigen receptor (CAR) T cell therapy, in which T cells are engineered to express a synthetic membrane receptor specific for a tumor antigen. CAR-T therapy has had a remarkable effect in patients with certain hematological malignancies (June, et al. Science 359, 1361-1365, doi:10.1126/science. aar6711 (2018); Majzner, et al. Nat Med 25, 1341-1355, doi:10.1038/s41591-019-0564-6 (2019)), with five CAR-T products currently approved by the U.S. Food and Drug Administration (FDA) for the treatment of multiple myeloma and B-cell malignancies (Upadhaya, et al. Nat Rev Drug Discov 20, 503-504, doi:10.1038/d41573-021-00100-z (2021)).

Engineered CAR T cell treatments of patients with cancer have shown promising clinical results. For example, genetically modified T cells expressing anti-CD19 CARs have recently been approved by the FDA for the treatment of patients with relapsed or refractory diffuse large B-cell lymphoma and B-cell acute lymphoblastic leukemia.

Despite the high response rates, relapses are observed in a large portion of patients due to a number of factors such as tumor antigen loss, T cell exhaustion, T cell dysfunction, and poor in vivo persistence.

Adoptive cellular immunotherapy with engineered chimeric antigen receptor (CAR) T cells has led to durable clinical responses in hematologic malignancies. Despite the high response rates, relapse occurs in a large proportion of patients due to a number of factors, including tumor antigen loss, T cell exhaustion, T cell dysfunction, and poor in vivo persistence of CAR-T cells (Orlando et al., Nat Med 24, 1504-1506 (2018), Majzner, Cancer Discov 8, 1219-1226 (2018)) of CAR-T cells. These are among the series of major challenges with CAR-T therapy, urging for better designs of CARs to overcome the therapeutic hurdles. Currently, the typical structure of CAR is composed of: (i) a single-chain variable fragment (scFv) for target recognition; (ii) a hinge for flexibility to access the target antigen; (iii) a transmembrane domain for cell surface anchoring; (iv) an intracellular domain containing costimulatory signaling domain(s) (which vary between the 1st, 2nd and 3rd generation of CARs); (v) and a T-cell receptor signaling domain (e.g., CD3zeta) for signal transduction (Jayaraman et al., EBioMedicine 58, 102931 (2020)). Compared with the 1st generation CAR, better clinical performances have been achieved in subsequent generations by incorporating CD28 or 4-1BB costimulatory signaling domains (Guedan et al., JCI Insight 3 (2018)). This demonstrates the importance of optimal CAR design. Weakening the signal strength of CAR by approaches like transient cession of CAR (Weber, et al., Science 372 (2021)) or adoption of low-affinity CAR (Ghorashian, et al., Nat Med 25, 1408-1414 (2019)) has been shown to greatly improve the efficacy of CAR-T cells. However, these approaches do not allow self-regulation of CAR thus lack flexibility.

To tackle the myriad challenges that different tumors and tumor environments present, multiple cell-based therapies besides CAR Ts have been generated, such as tumor infiltrating lymphocytes (TILs) (Verdegaal, et al. Nature 536, 91 95, doi:10.1038/nature18945 (2016)), T cell receptor T (TCR T) cells (Johnson, et al., Blood 114, 535 546, doi:10.1182/blood-2009-03-211714 (2009); Thomas, et al. Front Immunol 9, 947, doi:10.3389/fimmu.2018.00947 (2018)), CAR NK cells (Chu, et al. Leukemia 28, 917 927, doi:10.1038/leu.2013.279 (2014); Genssler, et al. Oncoimmunology 5, e1119354,(2016); Zhang, et al. J Immunol Res 2017, U.S. Pat. No. 6,915,912, (2017); Kruschinski, et al. Proc Natl Acad Sci USA 105, 17481-17486, doi:10.1073/pnas.0804788105 (2008); Liu, et al. Leukemia 32, 520 531, doi:10.1038/leu.2017.226 (2018); Chu, et al., Cancer Immunol Res 3, 333 344, doi:10.1158/2326-6066.CIR 14 0114 (2015); and Li, et al., Cell Stem Cell 23, 181 192 e185, doi:10.1016/j.stem.2018.06.002 (2018)), CAR macrophages (CAR Ms) (Klichinsky, et al., Nat Biotechnol 38, 947 953, doi:10.1038/s41587-020-0462-y (2020); Zhang, et al., J Hematol Oncol 13, 153, doi:10.1186/s13045-020-00983-2 (2020)), and human induced pluripotent stem cell (iPSC) derived therapeutic immune cells (Themeli, et al,. Nat Biotechnol 31, 928 933, doi:10.1038/nbt.2678 (2013)).

Additional components have been added to CAR constructs to enhance therapeutic efficacy or safety, e.g., kill switch CARs that can be depleted upon administration of a drug in the case of deleterious CAR toxicity (Di Stasi, et al. N Engl J Med 365, 1673 1683, doi:10.1056/NEJMoa1106152 (2011)), or tandem CARs that can target two different antigens (Shah, et al., Nat Med 26, 1569 1575, doi:10.1038/s41591-020-1081-3 (2020)).

Trogocytosis is a process by which lymphocytes such as T cells (Huang, et al., Science 286, 952-954 (1999)), B cells (Schriek, et al., Science 375, eabf7470 (2022)) and NK cells (Lu et al., Cancer Immunol Res 9, 1229-1241 (2021)) extract surface molecules from antigen presenting cells (APCs) through the immune synapse, and present those molecules on their own surface. Consequences of trogocytosis are context dependent. It has been reported that trogocytosis promotes the selection of high affinity cytotoxic T lymphocytes (CTL) by removing major histocompatibility complex (MHC)-peptide complex from antigen presenting cells (APCs) (Kedl, et al, Nat Immunol 3, 27-32 (2002)). However, it also leads to the elimination of reactive CTLs though fratricide when high amounts of MHC-peptide complexes are acquired and presented on their own surface. Recently, CAR-T cells have also been found to undergo trogocytosis, which causes tumor antigen loss and fratricide among themselves, compromising their therapeutic efficacy against cancer (Hamieh et al., Nature 568, 112-116 (2019); Olson et al., bioRxiv, 2021.2012.2005.471117 (2021); Rurik et al., Science 375, 91-96 (2022)). It is therefore important to develop new approaches to overcome CAR mediated trogocytosis for durable CAR-T therapeutic effect. Current approaches have shown that the combined targeting of tumor antigens or low affinity CAR is able to rescue CAR-T anti-tumor responses from trogocytosis-induced immune escape. However, these approaches are not adaptable or flexible, as they require generating entirely new scFVs for a broader range of each cancer types, when tumor associated antigens or choices of CAR are limited most of the time.

Therefore, there is a need for modified CAR-T cells that have improved retention of Tumor antigen and enhanced anti-cancer therapeutic efficacy.

Therefore, it is an object of the invention to provide enhanced CAR T cells having improved anti-tumor efficacy and methods thereof for treating proliferative diseases.

It is another object of the invention to provide compositions and methods for reducing trogocytosis, T cell exhaustion, and loss of tumor antigen among CAR T cells.

It is another object of the invention to provide CAR-T cells that retain a strong central memory phenotype.

It is a further object of the invention to provide CAR T cells that exhibit effective anti-tumor activity against a wide range of cancer types.

It is yet another object of the invention to provide systems for enhancing therapeutic activity of engineered cells of various immune cell types, including T cell, natural killer (NK) cell and cells in the myeloid lineage.

### SUMMARY OF THE INVENTION

Compositions and methods for improving the efficiency of CAR-T cells through engineering CARs for enhanced recycling at the cell surface have been developed. The disclosed compositions and methods are especially applicable to development of enhanced chimeric antigen receptor engineered T cell therapy (CAR-T).

Polypeptides including an amino acid sequence from the cytosolic domain of CTLA-4 and a heterologous amino acid sequence that is heterologous to CTLA-4 are provided. Typically, the polypeptides include between 25 and 41 contiguous amino acids of SEQ ID NO:3, or a functional fragment or variant thereof; and a heterologous amino acid sequence encoding a polypeptide that is heterologous to CTLA-4. Polypeptides including an amino acid sequence including at least 70% and less than 100% sequence identity to SEQ ID NO:3 or functional fragment thereof; and optionally a heterologous amino acid sequence that is heterologous to CTLA4 are also described. In some embodiments, the polypeptide further includes the amino acid sequence of SEQ ID NO:5, or a functional fragment or variant thereof. In some embodiments, the polypeptide includes the amino acid sequence of SEQ ID NO:7 or a functional fragment or variant thereof. In some embodiments, the polypeptide includes one or more additional copies of the amino acid sequence of SEQ ID NO:5, or a functional fragment or variant thereof. In some embodiments, the polypeptide includes the amino acid sequence of SEQ ID NO:51 or a functional fragment or variant thereof. In preferred embodiments, the amino acid sequence including the cytosolic domain of CTLA-4, is SEQ ID NO:3, or SEQ ID NO:7. A most preferred amino acid sequence including the cytosolic domain of CTLA-4 is SEQ ID NO:7. In some embodiments, the polypeptide also includes an additional amino acid sequence corresponding to any of SEQ ID NOs:5, or 9-50.

In preferred embodiments, the polypeptide can interact with clathrin adaptor activating protein 2 (AP-2). For example, in some embodiments, the polypeptide can co-immuno-precipitate with AP-2. In some embodiments, the polypeptide includes two YVKM amino acid motif(s).

Exemplary heterologous amino acid sequences further include one or more molecules such as a chimeric antigen receptor (CAR), programmed death protein 1 (PD1), protein transduction domain, fusogenic polypeptide, targeting signal, expression and/or purification tag. In some embodiments, the heterologous sequence includes a chimeric antigen receptor (CAR), and the polypeptide is present within the intracellular region of the CAR. In some embodiments, the heterologous sequence includes a chimeric antigen receptor (CAR), and the polypeptide is contiguous with the carboxyl terminus of the CAR. In some embodiments, the heterologous sequence includes a chimeric antigen receptor (CAR) having an intracellular component of CD3 zeta, and the polypeptide is contiguous with the intracellular component of CD3 zeta.

In some embodiments, the CAR is specific for an antigen selected from a cancer antigen, an inflammatory disease antigen, a neuronal disorder antigen, HIV/AIDS, a diabetes antigen, a cardiovascular disease antigen, an infectious disease antigen (such as a viral antigen, a protozoan antigen, a bacterial antigen, an allergen, etc.), an autoimmune disease antigen or an autoimmune disease antigen, or a combination thereof. Exemplary CAR targets are selected from AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR (3, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE. In some embodiments, the antigen is a cancer antigen selected from 4 1BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin 501, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin. In exemplary embodiments, the CAR is an anti-CD19 CAR or anti-CD22 CAR, or both. For example in particular embodiments, the CAR is CD19BBz-CAR or CD22(m971)-CAR.

Nucleic acids including a nucleic acid encoding the polypeptides are also described. Exemplary nucleic acids include a nucleic acid encoding a polypeptide including a chimeric antigen receptor (CAR) and one or more of SEQ ID NO:3, or SEQ ID NO:7, or SEQ ID NO:51. In some embodiments, the nucleic acid is RNA or DNA. In some embodiments, the nucleic acid is mRNA, and/or includes an expression control sequence(s), and/or is, or is encoded by a vector or a transposon. An exemplary vector is a viral vector, such as a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector including a combination of any two or more of a Adeno-associated virus (AAV) vector, Herpes Simplex virus (HSV) vector, vesicular stomatitis (VSV) vector, or a human Bocavirus vector (hBoV). In some embodiments, the vector is a nucleic acid expression vector selected from a plasmid, a cosmid, and a replicon. In some embodiments, the nucleic acid includes one or more of a promotor, a protein transduction domain, fusogenic polypeptide, or targeting signal conjugated thereto.

Isolated cells including the polypeptides and/or nucleic acids are also provided. Exemplary cells include a T cell, hematopoietic stem cell (HSC), macrophage, natural killer cell (NK), or dendritic cell (DC). For example, in some embodiments, the T cell is a CD8+ T cell selected from effector T cells, memory T cells, central memory T cells, and effector memory T cells. In other embodiments, the T cell is a CD4+ T cell selected from Th1 cells, Th2 cells, Th17 cells, and Treg cells. Isolated cells including a polypeptide encoding (i) an amino acid sequence encoding a CAR; and (ii) an amino acid sequence of one or more of SEQ ID NO:3, or SEQ ID NO:7, or SEQ ID NO:51, or a variant having at least 75% identity to SEQ ID NO:3, or at least 75% identity to SEQ ID NO:7, or at least 75% identity to SEQ ID NO:51, are also described. In preferred embodiments, the isolated cell includes a polypeptide including (i) an amino acid sequence encoding a CAR; and (ii) an amino acid sequence of SEQ ID NO:7, or a variant having at least 75% identity to SEQ ID NO:7. In some embodiments, the amino acid sequence of SEQ ID NO:7 is contiguous with the residue at the carboxyl terminus of the CAR.

Populations of cells derived by expanding an isolated cell including a polypeptide including: (i) an amino acid sequence encoding a CAR; and (ii) an amino acid sequence of SEQ ID NO: 7, or a variant having at least 75% identity to SEQ ID NO:7 are also provided. In some embodiments, the amino acid sequence of SEQ ID NO:7 is contiguous with the residue at the carboxyl terminus of the CAR.

Pharmaceutical compositions including (a) a population of cells expanded from an isolated cell including or expressing a polypeptide including: (i) an amino acid sequence encoding a CAR; and (ii) an amino acid sequence of SEQ ID NO:7, or a variant having at least 75% identity to SEQ ID NO:7; and (b) a pharmaceutically acceptable buffer, carrier, diluent or excipient are also provided.

Methods of treating a subject having a disease, disorder, or condition including administering to the subject an effective amount of a pharmaceutical composition of cells are also provided. Typically, the methods treat a subject having a disease, disorder, or condition associated with an elevated expression or specific expression of an antigen, by administering to the subject an effective amount of a T cell modified to include or express a polypeptide including: (i) an amino acid sequence encoding a CAR that targets the antigen; and (ii) an amino acid sequence of SEQ ID NO:7, or a variant having at least 75% identity to SEQ ID NO:7. In some embodiments, the cell was isolated from the subject having the disease, disorder, or condition. In other embodiments, the cell was isolated from a healthy donor.

Typically, the subject is a human, such as a human with a disease selected from cancer, an inflammatory disease, a neuronal disorder, HIV/AIDS, a diabetes, a cardiovascular disease, an infectious disease (including a viral, a protozoan, a bacterial disease, and an allergy), an autoimmune disease, or a genetic disorder.

In some embodiments, the subject has cancer or has been identified as being at increased risk of developing cancer.

Methods of introducing a fusion peptide including a CT domain having one or more YVKM motifs into a cell, are also provided. Typically, the method includes the steps of introducing to the cell: (i) a vector or transposon or mRNA encoding a polypeptide encoding a fusion peptide including a CT domain having one or more YVKM motifs; and (ii) causing the polypeptide to be expressed in the cell.

Chimeric antigen receptors (CARs), including one or more CT polypeptides (CAR-CT or CAR-CCT) are described. In certain embodiments, the CAR-CT targets CD22 and includes the amino acid sequence of any one of SEQ ID NOs:55, 57, 59. In a preferred embodiment, the chimeric antigen receptor (CAR) including a CT polypeptide (CAR-CT or CAR-CCT), targets CD22 and includes the amino acid sequence of SEQ ID NO:57. In other embodiments, the chimeric antigen receptor (CAR) including a CT polypeptide (CAR-CT), targets CD19 and includes the amino acid sequence of any one of SEQ ID NOs:61 63, 65. In a preferred embodiment, the chimeric antigen receptor (CAR) including a CT polypeptide (CAR-CT), targets CD19 and includes the amino acid sequence of SEQ ID NO:63. Nucleic acids, including a nucleic acid sequence encoding the chimeric antigen receptor including a CT polypeptide (CAR-CT), that targets CD19 and/or CD22 are also described. In some embodiments, the nucleic acid is a vector or a transposon. Isolated cells including a CAR including a CT polypeptide (CAR-CT) that targets CD19 and/or CD22, or a nucleic acid encoding the polypeptide are also described. Populations of cells derived from expanding the isolated cells including the CAR-CTs that target CD19 and/or CD22 are also provided, and pharmaceutical compositions including the populations of cells and a pharmaceutically acceptable buffer, carrier, diluent, or excipient are also described. Methods of treating a subject having a disease, disorder, or condition include administering to the subject an effective amount of the pharmaceutical composition. In some embodiments, the subject has cancer.

### BRIEF DESCRIPTION OF THE DRAWINGS

The accompanying drawings, which are incorporated in and constitute a part of this specification, illustrate several embodiments of the disclosed method and compositions and together with the description, explain the principles of the disclosed method and compositions.

FIG. 1 is a schematic representation of in vitro co-culture assay to assess trogocytosis between donor NALM6GL-PDL1-BFP cells (NALM6GL overexpressing PDL1-GGGS-BFP) and different groups of recipient NALM6 cells. Different experimental constructs depicted as inserted into NALM6 recipient cells each include: flanking (LTR) regions; EFS sequence (EFS); Flag sequence for detection of surface expression (Flag); protein construct (full length PD1 “fPD1”, PD1-Extracellular domain (PD1-ECD), PD1-Transmembrane “tPD1” (PD1-TM)); T2A sequences (2A) and optionally the cytoplasmic tail of CTLA-4 “PD1-CT” (CT); and fluorescent reporter (mScarlet), respectively. The amino acid sequence of the CT region from CTLA4 used in the constructs is: AVSLSKMLKKRSPLTTGVYVKMPPTEPECEKQFQPYFIPIN (SEQ ID NO:3)

FIG. 2 is a bar graph of quantification of PDL1 transfer from giver cells to receiver (recipient) cells measured by flow cytometry analysis, as indicated by BFP signal, showing % PDL1-BFP(0-100%) for each of fPD1(●), tPD1(▪) and PD1-CT(▴), respectively, in isotype, anti-PD1 and anti-PDL1, respectively.

FIG. 3 is a bar graph of quantification of surface PDL1 staining of receiver cells that have performed ligand uptake (gated on BFP+ mScarlet+ cells) PDL1-APC expression, showing % surface PDL1 (0-100%) for each of fPD1, tPD1 and PD1-CT, respectively. ****=p<0.0001.

FIG. 4 is a Schematic of in vitro co-culture assay to assess trogocytosis between donor NALM6GL-PDL1-BFP cells and different groups of recipient NALM6 cells, as in FIG. 1, but with recipient NALM6 cells infected by lentivirus carrying chimeric PD1 with intracellular domain replaced by one (PD1-CT), two (PD1-2CT) or three CTLA-4 cytoplasmic tails (PD1-3CT), followed by a fluorescent protein mScarlet.

FIG. 5 is a bar graph of quantification of PDL1 transfer, indicated by BFP signal, from donor cells to recipient cells, showing % PDL1-BFP (0-60%) for each of PD1-CT(●), PD1-2CT(Δ) and PD1-3CT(□), respectively, for each of isotype, anti-PD1 and anti-PDL1, respectively. Data are shown as mean±s.e.m. signal.

FIGS. 6A-6D are graphs showing representative flow cytometry results showing expression patterns of chimeric PD1 receptors PD1 receptors at the cell surface (Surface), and also cycling within the cell (Cycling), respectively, for each of samples including uninfected control (FIG. 6A), PD1-CT (FIG. 6B), tPD1 (FIG. 6C), and fPD1 (FIG. 6D), respectively.

FIG. 7 is a Schematic of staining procedures used in FIG. 6 for the detection of cycling and surface PD1 variants, showing antibody stain for PD1 using primary antibody labelled with a first label (some of which is then internalized into the cells), and a secondary antibody which is then used to detect any remaining primary antibody remaining at the cell surface.

FIG. 8 shows representative flow cytometry results expression patterns of chimeric PD1 in each of uninfected cells, fPD1, tPD1, PD1-CT, PD1-2CT, and PD1-3CT respectively, showing secondary antibody (4C/1 hr) over Primary Ab (37C/1 hr) for each receptor.

FIGS. 9A-9C are Schematics of the workflow used to generate CAR-T cells for effector function assessment in vitro, showing: constructs of CARs engineered with either single (22CAR-1CT), tandem (22CAR-2CT) or triplex CTs (22CAR-3CT) fused to the C-terminal of human CD22 CAR (22CAR), followed by a fluorescent reporter mScarlet, separated by T2A sequences with Flag sequences at N-terminal of the CAR for surface expression detection; and generation of CAR-T cells, with human CD3 T cells infected by lentiviruses carrying CAR targeting human CD22 (M971-BBz) (FIG. 9A); Round 1 coculture with those four groups of CAR-T cells generated in FIG. 9A cocultured with NALM6GL cells at variable E/T ratios for 24 hours (FIG. 9B); and Round 2 coculture, with an additional round of NALM6GL cells were supplemented into the round 1 coculture for 24 hours, then FACS analysis with cells from both round1 and 2 cocultures were quantified by fluorescence-activated single cell sorting (FACS) (FIG. 9C).

FIGS. 10A-10W are graphs of the of function CAR T cells with each of CD22 CAR (22CAR), CD22 CARs engineered with either single (22CAR-1CT), tandem (22CAR-2CT) or triplex CTs (22CAR-3CT) fused to the C-terminal, respectively, showing: Quantification of flow cytometry analysis for surface expression level indicated by Flag staining in medium fluorescent intensity (0-6,000 Flag MFI) (FIG. 10A); Quantification of the increases in CAR T cell counts after one round of NALM6GL stimulation, showing % CAR T cells increase (normalized with input) (FIG. 10B); Quantification of NALM6GL percentage (0-100 NALM6(%)) after 2 rounds of NALM6GL stimulation of 22CAR(●), 22CAR-1CT(▪), 22CAR-2CT(Δ) or 22CAR-3CT( [symbol] ) (FIG. 10C); Quantification of NALM6GL counts (0-200 NALM6 counts (×1000)) after 2 rounds of NALM6GL stimulation of 22CAR(●), 22CAR-1CT(▪), 22CAR-2CT(Δ) or 22CAR-3CT( [symbol] ) (FIG. 10D); Quantification of 22CAR-T cell percentage (0-100 CAR T (%)) after 2 rounds of NALM6GL stimulation of 22CAR(●), 22CAR-1CT(▪), 22CAR-2CT(Δ) or 22CAR-3CT( [symbol] ) (FIG. 10E); Quantification of 22CAR-T cell counts (0-100 CAR T counts (×1,000)) after 2 rounds of NALM6GL stimulation of 22CAR(●), 22CAR-1CT(▪), 22CAR-2CT(Δ) or 22CAR-3CT( [symbol] ) (FIG. 10F); Quantification of representative flow cytometry results showing % NALM6 killing by CAR T cells (FIG. 10G); Quantification of flow cytometry results showing the expression level of LAG3 on CAR T cells (0-80% LAG3+) after being cocultured with one round of NALM6GL cells (FIG. 10H); and Quantification of input CAR-T cell counts (0-150 CAR T counts (×1,000)) without NALMG6GL stimulation (FIG. 10I), respectively. ns=p>0.05, *=p<0.05, **=p<0.01, ***=p<0.001, ****=p<0.0001. FIGS. 10J-10L are graphs showing % pERK1/2 (pT202/pY204) (FIG. 10J); % IFNg (FIG. 10K); and % TNFa (FIG. 10L), for each of CAR (●), CAR-1CT (1-CCT; ▴), CAR-2CT (2-CCT; ▪) or CAR-3CT (3-CCT; ∘), respectively. FIG. 10M is a graph showing Percentage (%) for each of CAR (●), CAR-1CT (1-CCT; ▴), CAR-2CT (2-CCT; ▪) or CAR-3CT (3-CCT; ∘), respectively, for each of Surface, Endocytic and CAR-cells, respectively. FIGS. 10N-10Q are line graphs showing Relative recycling CAR over Staining duration of 2° Ab at 37° C. (min) (FIG. 10N): inhibition of protein synthesis (50 μg/ml cyclohexamide) for relative total CAR over time (0-4 hr) (FIG. 10O); Inhibition of lysosome degradation (10 nM BafA1) for relative CAR over time (0-4 hr) (FIG. 10P); and Inhibition of proteasome degradation (Bortezomib 10 μM) for relative CAR over time (0-4 hr) (FIG. 10Q) for each of CAR, CAR-1CT (1CCT), CAR-2CT (2CCT) or CAR-3CT (3-CCT), respectively.

FIG. 10R is a graph showing % CAR-T population (0-60%) of each of CD4 and CD8 cells for each of CAR (●), CAR-1CT (1-CCT; ▴), CAR-2CT (2-CCT; ▪) or CAR-3CT (3-CCT; ∘), respectively. FIGS. 10S-10V are graphs showing % LAG-3 (FIG. 10S); % PD-1 (FIG. 10T); % TIGIT (FIG. 10U); % cycling CTLA-4 (FIG. 1V), respectively, for each of CAR (●), CAR-1CT (1-CCT; ▴), CAR-2CT (2-CCT; ▪) or CAR-3CT (3-CCT; ∘), respectively. FIG. 10W is a graph showing Relative recycling CTLA-4 over time (0-1 hr) for each of CAR, CAR-1CT (1CCT), CAR-2CT (2CCT) or CAR-3CT (3-CCT), respectively.FIGS. 11A-11L show trogocytosis and fratricide of CAR T cells with CTLA-4 cytoplasmic tail fusion.

FIG. 11A is a schematic demonstrating trogocytosis activity and presence of tumor antigen amongst 22CAR-T cells (22CAR), CD22 CARs engineered with either single (22CAR-1CT), tandem (22CAR-2CT) or triplex CTs (22CAR-3CT) fused to the C-terminal, respectively, and NALM6GL cells); FIGS. 11B-11E are bar graphs showing: Quantification of flow cytometry results of CD22 expression (CD22 (0-50%)) on CAR T cells after 1 hour (1 hr), 2 hr and 4 hr, for each of 22CAR(●), 22CAR-1CT(▪), 22CAR-2CT(Δ) or 22CAR-3CT( [symbol] ), respectively (FIG. 11B); Quantification of flow cytometry results of CD22 expression (CD22^{low} (0-50%)) on NALM6GL cells after 1 hour (1 hr), 2 hr and 4 hr, for each of 22CAR(●), 22CAR-1CT(▪), 22CAR-2CT(Δ) or 22CAR-3CT( [symbol] ), respectively (FIG. 11C); and Quantification of flow cytometry results showing the differentiation stage, indicating by the expression of CD45RO and CD62L, of CAR-T cells (CD45RO+ CD62L+(0-60%)) (FIG. 11D); and Quantification of flow cytometry results showing the expression level of LAG3 on CAR-T (LAG3+(0-80%)) cells after being cocultured with two rounds of NALM6GL stimulation (FIG. 11E), for each of 22CAR, 22CAR-1CT, 22CAR-2CT, and 22CAR-3CT, respectively. ns=p>0.05, *=p<0.05, **=p<0.01, ***=p<0.001, ****=p<0.0001. FIG. 11F is a graph showing quantification of mCD22 on Trog+ CAR T-cells over Membrane CD22 (0-100%), for each of CAR, CAR-1CT (1CCT), CAR-2CT (2CCT) or CAR-3CT (3-CCT), respectively. FIG. 11G is a graph showing % CD107a (0-15%) of CAR, CAR-1CT (1CCT), CAR-2CT (2CCT) or CAR-3CT (3-CCT) CAR-J cells, for groups of CAR-T:Trog⁻ CAR-J=1:1 (●); CAR-T:Trog⁺ CAR-J=1:0.5 (▴); and CAR-T:Trog⁺ CAR-J=1:1 (▪), respectively. FIG. 11H is a graph showing % LAG-3⁺ (0-80%); for each of CAR (●), CAR-1CT (1-CCT; ▴), CAR-2CT (2-CCT; ▪) or CAR-3CT (3-CCT; ∘), respectively. FIG. 11I is a graph CD22 MFI (0-2500); for each of CD22^{high} and CD22^{low} groups, respectively. FIG. 11J is a graph showing NALM6 counts (×1,000) for each of CAR (●), CAR-1CT (1-CCT; ▴), CAR-2CT (2-CCT; ▪) or CAR-3CT (3-CCT; ∘), respectively, for each of CD22^{high} and CD22^{low} groups, respectively. FIG. 11K is a graph showing % GFP+CAR-T (0-40%) for each of CAR (●), CAR-1CT (1-CCT; ▴), CAR-2CT (2-CCT; ▪) or CAR-3CT (3-CCT; ∘), respectively, for each of DMSO and Latrunculin A groups, respectively. FIG. 11L is a graph showing % BFP⁺CAR-J (0-20%) over time (0, 1, 2, and 4 hr, respectively).

FIGS. 12A-12E show transcriptome profiling of engineered CAR T cells with CTLA-4 cytoplasmic tail. FIG. 12A is a schematic of constructs of CD19 CAR (19CAR), engineered with either single (19CAR-1CT), tandem (19CAR-2CT) or triplex CTs (19CAR-3CT) fused to the C-terminal of human CD19 CAR, followed by a fluorescent reporter mScarlet, separated by T2A sequences, with Flag sequences tagged at N-terminal of the CAR for surface expression detection. FIGS. 12B-12E are bar graphs showing: Quantification of 19CAR-T cell percentage (CAR T (%)) (FIG. 12B); Quantification of 19CAR-T cell counts (CAR T counts (×1000)) (FIG. 12C); Quantification of NALM6GL percentage (NALM6(%))(FIG. 12D); and Quantification of NALM6GL counts (NALM6 counts (×1000)) (FIG. 12E) for each of CD19 CAR (19CAR), engineered with either single (19CAR-1CT), tandem (19CAR-2CT) or triplex CTs (19CAR-3CT), respectively. ns=p>0.05, *=p<0.05, **=p<0.01, ***=p<0.001, ****=p<0.0001.

FIG. 13A is a schematic of the work flow of a fratricide assay, showing CAR, CAR-1CT, CAR-2CT or CAR-3CT cells labeled with either 1 μM or 10 μM eflour450 and mixed at 1:1 ratio. These mixed CAR T cells were then cocultured with or without NALM6GL cells (1:1 ratio) at E/T=1/2 for 24 hours. FACS analysis is then used to identify relative percentages of eflour450 high and low populations gated from CAR+ (mScarlet+) cells were used for quantification of relative survival of CAR-T cells. Relative resistance to fratricide (%)=[(lw:hw)−(lwo:hwo)]/(Iwo:hwo)*100%, (in which lw and hw stands for percentage of eflour450 low and high population, respectively, with NALM6GL cells stimulation, while lwo and hwo stands for percentage of eflour450 low and high population, respectively, without NALM6GL cells stimulation). FIGS. 13B-13C are bar graphs showing quantification of relative survival of 19CAR-T cells (% Relative survival Normalized with input) for 19CAR, 19CAR-1CT, 19CAR-2CT or 19CAR-3CT (FIG. 13B); and 22CAR, 22CAR-1CT, 22CAR-2CT or 22CAR-3CT (FIG. 13C), respectively, from fratricide assay described in FIG. 13A. FIG. 13D is a graph showing % divided cells (0-25%) of each of CAR (●), CAR-1CT (1-CCT; ▴), CAR-2CT (2-CCT; ▪) or CAR-3CT (3-CCT; ∘), respectively, in each of division numbers 1, 2, 3, 4, and 5, respectively. FIG. 13E is a graph showing % Annexin V+ (0-60%) for each of CAR (●), CAR-1CT (1-CCT; ▴), CAR-2CT (2-CCT; ▪) or CAR-3CT (3-CCT; ∘), respectively, in each of Trog+ and Trog− groups, respectively. FIG. 13F is a schematic of in vivo experimental workflow used in b-f. NSG mice (female, 6-10 weeks old) were first inoculated with 1 million NALM6GL cells intravenously (i.v.) on day 0. CAR-T cells stained with 1 μM eFluor450 were mixed with CAR-T cells stained with 10 μM eFluor450 at 1:1 ratio. Two million of these labeled cell mixtures were transferred to the leukemia NSG models on day 4. One day post CAR-T transfer, bone marrow samples were collected for flow cytometry analysis to determine the relative percentage of eFluor450^{low} and eFluor450^{high} populations. FIGS. 13G-13H are graphs showing relative survival (FIG. 13G); and % CD22⁺CAR-T (FIG. 13H) for each of CAR, CAR-1CT (1CCT), CAR-2CT (2CCT) or CAR-3CT (3-CCT), respectively. FIG. 13I is a graph showing % CD22+CAR-T cells (0-40%) over % relative survival for each of CAR (●), CAR-1CT (1-CCT; ▪), CAR-2CT (2-CCT; ▴) or CAR-3CT (3-CCT; ▾), respectively. FIG. 13J is a graph showing CAR-T/NALM6GL ratio % for each of CAR (●), CAR-1CT (1-CCT; ▴), CAR-2CT (2-CCT; ▪) or CAR-3CT (3-CCT; ∘), respectively.

FIGS. 14A-14B are graphs of pathway analysis by over representation analysis (ORA) showing top 10 pathways of genes up-regulated in 22CAR-2CT vs. 22CAR (FIG. 14A) and in 22CAR-3CT vs. 22CAR (FIG. 14B). FIG. 14C is a Venn diagram showing overlaps of upregulated genes expressed in 22CAR-1CT vs. 22CAR, 22CAR-2CT vs. 22CAR and 22CAR-3CT vs. 22CAR. Overlapping genes are indicated.

FIG. 15A is a schematic depicting the in vivo experimental workflow, showing NSG mice (6-10 weeks old) inoculated with 0.5 million NALM6GL cells intravenously (0.5 M NALM6GL i.v.) on day 0 and treated with 1 million CAR T cells (1M CAR-T i.v.) on day 4; mice are re-challenged with 0.5 million NALM6GL cells (rechallenge 0.5 M NALM6GL i.v.) on day 12; 2.5 μg human recombinant IL2 is administrated subcutaneously (s.c.) daily starting on day 4 for 24 days; Disease progression is monitored by bioluminescence imaging and survival analysis. FIGS. 15B-15C are graphs showing quantification of bioluminescence imaging, with Tumor burden measured by total radiance (p/sec/cm2/sr) over time (0-50 days post tumor inoculation) (FIG. 15B); and Probability of survival (0-100) over time (0-80 days post inoculation) (FIG. 15C); for each of mock T (●), 22CAR (▪), 22CAR-1TC (▴); 22CAR-2CT (▾); and 2CAR-3CT (♦), respectively.

FIG. 16A is plot of the quantification of 13 granule molecules and cytokines (IL4, IL6, IL2, TNF-a, IL17A, sFASL, IFNg, GranzymeB, sFas, GranzymeA, IL10, Perforin and Granulysin) in CAR T and NALM6GL coculture supernatant measured for each of 22CAR, 22CAR-1TC; 22CAR-2CT; and 2CAR-3CT, respectively. FIG. 16B is a bar graph showing quantification of proliferating cells (%) for each of 22CAR, 22CAR-1CT, 22CAR-2CT or 22CAR-3CT, respectively. Data are shown as mean±s.e.m. One-way ANOVA with Dunnett's multiple-comparisons test is used to assess significance. ns=p>0.05. FIGS. 16C-16D are graphs showing CAR-T counts (×1,000) (FIG. 16C); and % CD45RO⁺CD62L⁺ (0-15%) (FIG. 16D) for each of CAR (●), CAR-1CT (1-CCT; ▴), CAR-2CT (2-CCT; ▪) or CAR-3CT (3-CCT; ∘), respectively.

FIGS. 17A-17B are graphs showing Principal component analysis (PCA), showing all four groups of 22CAR-T cells (22CAR, 22CAR-1CT, 22CAR-2CT or 22CAR-3CT, respectively) at baseline without stimulation (PC2: 25% variance/PC1:42% Variance) (FIG. 17A); and for all four groups of 22CAR-T cells with 2 rounds of NALM6GL stimulation (PC2: 2% variance/PC1: 94% Variance) (FIG. 17B).

FIGS. 18A-18C are bar graphs of quantification of baseline expression levels on each of 22CAR, 22CAR-1CT, 22CAR-2CT or 22CAR-3CT, respectively, showing LAG3+(%) (FIG. 18A); HHLA2+(%) (FIG. 18B); and CD101+(%) (FIG. 18C) for each group, respectively. Data are shown as mean±s.e.m. One-way ANOVA with Dunnett's multiple-comparisons test is used to assess significance. ns=p>0.05, *=p<0.05, **=p<0.01, ***=p<0.001, ****=p<0.0001.

FIG. 19A is a schematic depicting the in vivo phenotyping of CAR-T cells. NSG mice (6-10 weeks old) were inoculated with 0.5 million NALM6GL cells intravenously (0.5 M NALM6GL i.v.) on day 0 and treated with 1 million CAR T cells (1M CAR-T i.v.) on day 4; mice are re-challenged with 0.5 million NALM6GL cells (rechallenge 0.5 M NALM6GL i.v.) on day 12; 2.5 μg human recombinant IL2 is administrated subcutaneously (s.c.) daily from day 4 to day 14. On day 15, bone marrow and spleen of each mouse were collected for flow cytometry analysis. FIGS. 19B-19C are graphs showing quantification of flow cytometry results showing abundance of CAR-T cells and NALM6GL cells in Spleen (FIG. 19B); and quantification of flow cytometry results showing CD45RO and CD62L expression (CD45RO+CD62L+%) of CAR-T cells in bone marrow (FIG. 19C) for each of 22CAR, 22CAR-1TC, 22CAR-2CT, and 2CAR-3CT, respectively. ns=p>0.05, *=p<0.05, **=p<0.01, ***=p<0.001, ****=p<0.0001.

FIG. 20 is a schematic of the proposed model of titrating optimal CAR-T function with CCT fusion, showing CAR fused with duplex CCTs (CAR-2CCT) demonstrated decreased surface expression, which is tightly regulated by its endocytosis, recycling, degradation in both lysosome and proteasome. Compared with the control CAR, CAR-2CCT has significant decreases in surface CAR expression, T cell activation, CAR-mediated trogocytosis, and causes less tumor antigen loss on NALM6 cells. This engineering approach effectively increases the persistence and proportion of Tcm cells in CAR-2CCT cells, leading to a remarkable improvement in their anti-tumor in vivo.

### DETAILED DESCRIPTION OF THE INVENTION

The disclosed method and compositions can be understood more readily by reference to the following detailed description of embodiments and the Examples included therein and to the Figures and their previous and following description.

Utilizing the endocytic feature of cytotoxic T-lymphocyte-associated antigen-4 (CTLA-4) cytoplasmic tails (CTs), CAR function was reprogramed to substantially enhance CAR-T efficacy in vivo. CARs with monomeric, duplex, or triplex CTs fused to the C-terminus demonstrated progressively reduced trogocytosis, fratricide among CAR-T cells, and tumor antigen loss. CAR with duplex CTs (CAR-2CT) showed superior anti-tumor efficacy in vivo in a relapsed leukemia model. Transcriptome profiling revealed unbiased gene expression programs of CAR-CT T cells, such as lower tonic signaling at baseline and durable responsiveness to repeated stimulations. Immunological characterization demonstrated that CAR-2CT cells retain a stronger central memory phenotype and were more persistent. This system provides a distinct approach for engineering therapeutic T cells and enhancing CAR-T function by synthetic CT fusion, which is orthogonal to other cell engineering systems.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

As used herein, “homologous” means derived from a common ancestor. For example, a homologous trait is any characteristic of organisms that is inherited by two or more species from a common ancestor species. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

“Heterologous” means having a different relation, relative position, or structure. Thus, unless otherwise specified, heterologous includes joining or linking of two or more amino acid or nucleic acid sequences from that organism (e.g., species) that are not normally found joined or linked (e.g., together) as well as joining or linking of two or more amino acid or nucleic acid sequences from different species.

“Endogenous” refers to any material from or produced inside an organism, cell, tissue or system.

“Exogenous” refers to any material introduced from or produced outside an organism, cell, tissue or system.

The term “Chimeric Antigen Receptor”, or alternatively “CAR”, refers to a set of polypeptides, typically two in the simplest embodiments, which when in an immune effector cell, provides the cell with specificity for a cancer cell, or other specific cell, and with intracellular signal generation. In exemplary embodiments, a CAR includes at least an antigen binding domain such as an extracellular binding domain, a transmembrane domain and a cytoplasmic signaling domain (also referred to as “an intracellular signaling domain”) including a functional signaling domain derived from a stimulatory molecule and/or costimulatory molecule. The term “antigen binding domain” is used in the context of a CAR to refer to the portion of a CAR that specifically recognizes and binds to an antigen of interest. The “antigen binding domain” of a CAR may be derived from a binding protein such as an antibody or fragment thereof. In some embodiments, the “binding domain” of a CAR is a single-chain variable fragment (scFv). In certain embodiments, the “binding domain” of a CAR includes the complementarity determining regions of a binding protein disclosed herein. In some embodiments, the stimulatory molecule is, or is derived from, the CD3ƒ ((zeta), also known as “zeta stimulatory domain”, associated with a T cell receptor complex. In some embodiments, the cytoplasmic signaling domain of the CAR further includes one or more functional signaling domains derived from at least one costimulatory molecule (e.g., 4-1BB (i.e., CD137), CD27 and/or CD28). In some embodiments, the CAR includes a chimeric fusion protein including an extracellular antigen binding domain, a transmembrane domain and an intracellular signaling domain including a functional signaling domain derived from a stimulatory molecule. In various embodiments, CARs are fusion proteins of single-chain variable fragments (scFv) fused to a CD3-zeta transmembrane domain. However, other intracellular signaling domains such as CD28, 41-BB and O×40 may be used in various combinations to give the desired intracellular signal. Exemplary CAR-T cells include Axicabtagene ciloleucel (KTE-C19, Axi-cel), Tisagenlecleucel, Lisocabtagene Maraleucel (liso-cel; JCAR017).

The term “antigen” as used herein is defined as a molecule capable of being bound by an antibody or T-cell receptor. An antigen can additionally be capable of provoking an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which includes a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the disclosed compositions and methods includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. In the context of cancer, “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are tumor specific antigens (TSA) which are present only on tumor cells and not on healthy cells, as well as tumor associated antigens (TAA) which are present in tumor cells and on some normal cells. In some forms, the chimeric antigen receptors are specific for tumor specific antigens. In some forms, the chimeric antigen receptors are specific for tumor associated antigens. In some forms, the chimeric antigen receptors are specific both for one or more tumor specific antigens and one or more tumor associated antigens.

The terms “CD3ƒ”, “CD3 zeta” or “CD3 eta” are used interchangeably to define the protein provided as GenBan Acc. No. BAG36664.1, or the equivalent residues from a non-human species. A “zeta stimulatory domain” or alternatively a “CD3-zeta stimulatory domain” is defined as the amino acid residues from the cytoplasmic domain of the zeta chain, or functional derivatives thereof, that are sufficient to functionally transmit an initial signal necessary for T cell activation.

The term “immune effector cell,” is used herein to refer to a cell that is involved in an immune response (e.g. promotion of an immune effector response). Examples of immune effector cells include T cells, e.g., alpha/beta T cells and gamma/delta T cells, B cells, natural killer (NK) cells, natural killer T (NKT) cells, mast cells, and myeloic-derived phagocytes. In some embodiments, the immune effector cell(s) is allogenic. In some embodiments, the immune effector cell(s) is autologous. Immune effector cells such as T cells may be activated and expanded generally using methods previously described, such as for example, as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, all of which are incorporated herein by reference in their entirety.

“Bi-specific chimeric antigen receptor” refers to a CAR that includes two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” or “functional variant” means a fragment or variant of a polypeptide, such as a full-length or native polypeptide, that retains one or more functional properties of the full-length or native polypeptide. For example, in some embodiments, a functional fragment or functional variant of a CT polypeptide is a fragment or variant that retains the function of binding to the Clathrin adaptor activating protein 2 (AP-2).

As used herein, the terms “variant” or “active variant” refers to a polypeptide or polynucleotide that differs from a reference polypeptide or polynucleotide, but retains one or more functional properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the disclosure.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

It has been established that polypeptides from the intracellular domain of the CTLA-4 gene product impart the function of enhanced endosomal cycling at the cell surface. Compositions including isolated polypeptides (“CT” or “CTT” polypeptides) from the intracellular domain of the CTLA-4 (“CTLA-4 tail”), and fusion proteins incorporating the CT polypeptides together with one or more heterologous sequences are provided. Chimeric Antigen Receptors (CAR) including one, two, three or more CT polypeptides are provided. In preferred embodiments, the CAR-CT fusion peptides include 2 copies of CT in tandem. Recombinant constructs including nucleic acids expressing or encoding the polypeptides and fusion proteins are also provided. Viral genomes including the recombinant constructs, recombinant viruses including the constructs, and vaccine formulations formed thereof are also provided.

### A. CTLA4 Cytoplasmic Domain (CTLA-4 Tail) Polypeptides

CT polypeptides that enhance cell cycling are described as components of fusion proteins including the CT polypeptide and heterologous polypeptide sequences.

Compositions including and encoding a polypeptide including a fragment of the cytoplasmic “tail” domain of a CTLA-4 gene product or variant thereof, referred to herein as “CT polypeptides” and alternatively as “CCT polypeptides”, are provided. As discussed in more detail below, the CT polypeptides include between 10 and 100 contiguous amino acids and include all or part of the cytoplasmic tail component of a CTLA-4, e.g., endogenous human CTLA-4, or homolog, or a functional fragment or variant thereof. The CT polypeptides, nucleic acids encoding the same, and delivery vehicles thereof and cells including them can optionally include one or more additional heterologous proteins, polypeptides or other amino acid sequences. Typically, the presence of one or more CT polypeptides within the cytoplasmic domain of a recombinant fusion peptide will impart an increased cell-cycling function to the fusion peptide. Thus, in some embodiments CT domains are described as part of a fusion protein. As discussed in more detail below, the fusion proteins can be or include one or more an extracellular domain(s), transmembrane domain(s), and/or cytoplasmic/intracellular domain(s). One or more CT polypeptides can form a part or all of one or more of one or more the extracellular domain(s), transmembrane domain(s), and/or cytoplasmic/intracellular domain(s). CT polypeptides can also be expressly excluded from one or more the extracellular domain(s), transmembrane domain(s), and/or cytoplasmic/intracellular domain(s). CT polypeptides of each domain can be alone or in combination with one or more heterologous sequences. The portion(s) of the fusion protein heterologous to the CT polypeptide(s) can be or include one or more of extracellular domain(s), transmembrane domain(s), and cytoplasmic/intracellular domain(s). Likewise, heterologous sequence can be expressly excluded from one or more of the extracellular domain(s), transmembrane domain(s), and cytoplasmic/intracellular domain(s).

### 1. Cytotoxic T-Lymphocyte-Associated Antigen-4 (CTLA-4)

Cytotoxic T-lymphocyte-associated antigen-4 (“CTLA-4”, or “CTLA4”, also known as CD152) is an immune checkpoint molecule, which is important for maintaining self-tolerance and homeostasis because the deletion of CTLA-4 leads to systemic autoimmune diseases (Tivol et al., Immunity 3, 541-547 (1995)). CTLA4 is a member of the immunoglobulin superfamily and is a costimulatory molecule expressed by activated T cells. CTLA4 protein is a homodimer interconnected by a disulfide bond in the extracellular domain at cysteine residue 120. Each monomeric polypeptide contains a high affinity binding site for the costimulatory molecules B7-1 and B7-2. CTLA4 is similar to the T-cell costimulatory CD28, and both molecules bind to B7-1 (CD80) and B7-2 (CD86) on antigen-presenting cells. CTLA4 transmits an inhibitory signal to T cells, whereas CD28 transmits a stimulatory signal.

CTLA4 binds to the B7 isoforms with an affinity that is 10 to 100 times that of CD28. The crystal structure of CTLA-4 is described in Ostrov, et al., “Structure of murine CTLA-4 and its role in modulating T cell responsiveness.” Science 290: 816-819, 2000, which is incorporated herein by reference in its entirety). Consistent with its membership in the Ig superfamily, CTLA-4 displays a strand topology similar to V-alpha domains. CTLA-4 has an unusual dimerization mode that places the B7 binding sites distal to the dimerization interface, allowing each CTLA-4 dimer to bind 2 divalent B7 molecules. The periodic rearrangement of these components might explain the role of CTLA4 in the regulation of T-cell responsiveness.

Human CTLA-4 maps to chromosome 2q33; the deduced 223-amino acid human protein has a mass of 24,626 Da and shows high homology (76%) to the corresponding mouse protein. CTLA-4 is a membrane-bound, cell surface receptor having an extracellular domain, transmembrane domain, and intracellular cytoplasmic domain. The extracellular domain includes amino acids at positions 36-161 of 223; the transmembrane domain includes amino acids at positions 162-182 of 223, and the cytoplasmic domain includes amino acids at positions 183-223 of 223. As used herein, a “transmembrane domain” refers to any protein structure that is thermodynamically stable in a cell membrane, preferably a eukaryotic cell membrane. As used herein, the terms “extracellular domain” and “ectodomain” refer to any protein structure that is thermodynamically stable in outside of the cell membrane (i.e., in the extracellular space). As used herein, an “intracellular domain” or “cytoplasmic domain” refers to any protein structure that is thermodynamically stable in inside of the cell membrane (i.e., in the intracellular cytosol).

An exemplary consensus amino acid sequence for the mature human CTLA-4 protein (UniProtKB accession number: Q6GR94 (Q6GR94_HUMAN)) is:

(SEQ ID NO: 1)
MACLGFQRHKAQLNLAARTWPCTLLFFLLFIPVFCKAMHVAQPAVVLAS
SRGIASFVCEYASPGKATEVRVTVLRQADSQVTEVCAATYMMGNELTFL
DDSICTGTSSGNQVNLTIQGLRAMDTGLYICKVELMYPPPYYLGIGNGT
QIYVIDPEPCPDSDFLLWILAAVSSGLFFYSFLLTAVSLSKMLKKRSPL
TTGVYVKMPPTEPECEKQFQPYFIPIN.

Amino acids within the transmembrane domain are represented in bold font and amino acids within the cytoplasmic domain are underlined.

An exemplary mRNA sequence for the human CTLA-4 gene expression product is:

| (SEQ ID NO: 2) | |
|---|---|
| 1 | atggcttgcc ttggatttca gcggcacaag gctcagctga acctggctgc caggacctgg |
| 61 | ccctgcactc tcctgttttt tcttctcttc atccctgtct tctgcaaagc aatgcacgtg |
| 121 | gcccagcctg ctgtggtact ggccagcagc cgaggcatcg ccagctttgt gtgtgagtat |
| 181 | gcatctccag gcaaagccac tgaggtccgg gtgacagtgc ttcggcaggc tgacagccag |
| 241 | gtgactgaag tctgtgcggc aacctacatg acggggaatg agttgacctt cctagatgat |
| 301 | tccatctgca cgggcacctc cagtggaaat caagtgaacc tcactatcca aggactgagg |
| 361 | gccatggaca cgggactcta catctgcaag gtggagctca tgtacccacc gccatactac |
| 421 | ctgggcatag gcaacggaac ccagatttat gtaattgatc cagaaccgtg cccagattct |
| 481 | gacttcctcc tctggatcct tgcagcagst agttcggggt tgttttttta tagctttctc |
| 541 | ctcacagctg tttctttgag caaaatgcta aagaaaagaa gccctcttac aacaggggtc |
| 601 | tatgtgaaaa tgcccccaac agagccagaa tgtgaaaagc aatttcagcc ttattttatt |
| 661 | cccatcaatt ga. |

CTLA-4 shares 2 ligands, CD80 and CD86, with a stimulatory receptor, CD28. Whereas CD28 is found on the membrane of resting T cells, CTLA-4 is detectable only on cells activated after antigen presentation. CTLA-4 can capture its ligands and CD86 from opposing cells by a process of trans-endocytosis (Qureshi, et al. Science 332: 600-603, 2011). After removal, these costimulatory ligands are degraded inside CTLA4-expressing cells, resulting in impaired co-stimulation via CD28. Acquisition of CD86 from antigen-presenting cells is stimulated by T cell receptor engagement and observed in vitro and in vivo.

Mechanistically, it was first reported that the inhibitory signal was transduced by the cytoplasmic tail of CTLA-4, namely by cell-intrinsic inhibition (Walunas, et al., Immunity 1, 405-413 (1994)). However, accumulating evidence demonstrates that the dominant role of CTLA-4 is achieved through cell-extrinsic inhibition, in which CTLA-4 is able to trogocytose costimulatory molecules CD80/CD86 expressed on APCs, thereby inhibiting activation of naïve T cells through CD28 signaling (Bachmann, et al., J Immunol 163, 1128-1131 (1999); Qureshi et al., Science 332, 600-603 (2011); Tekguc, et al., Proc Natl Acad Sci USA 118, (2021); and Corse, et al., J Immunol 189, 1123-1127 (2012)).

a. YVKM Motif

The mechanism of immune regulation in which CTLA-4 acts as an effector molecule to inhibit CD28 co-stimulation by the cell-extrinsic depletion of ligands, accounts for many of the features of the CD28-CTLA4 system. One distinct feature of CTLA-4 is that it is highly endocytic with the majority of expressed CTLA-4 being constantly cycled between cell surface and intracellular compartments. This occurs through the interaction with the Clathrin adaptor activating protein 2 (AP-2) by the amino acid sequence YVKM (SEQ ID NO:86) within the cytoplasmic domain of CTLA-4 (corresponding to amino acids 201-204 of SEQ ID NO:1), resulting in limited surface expression of CTLA-4. Therefore, rather than to direct transduce suppressive signals, the cytoplasmic component of CTLA-4 is believed to regulate the surface availability of CTLA-4 for optimal T cell activation Walker and Sansom, Nat Rev Immunol 11, 852-863 (2011)). The terms “YVKM (SEQ ID NO:86)”, “YVKM sequence”, “YVKM motif” and “YVKM domain” are used interchangeably to refer to the component of the CTLA-4 cytoplasmic domain that interacts with AP-2, corresponding to amino acids 201-204 of SEQ ID NO:1.

### 2. CTLA-4 Tail and CT Sequences

Isolated fragments of the cytoplasmic component of CTLA-4 are disclosed. The complete cytoplasmic domain of CTLA-4 is termed a “CTLA-4 tail”, for example, including all of the amino acids at positions 182-223 of SEQ ID NO:1. The terms “CT sequence”, “CT polypeptide”, and “CT peptide”, are used interchangeably to refer to the polypeptide that is or includes from 5 to 40, inclusive, contiguous amino acids from within the intracellular (cytoplasmic) CTLA-4 tail, and functional fragments and variants thereof.

In some embodiments, the CT polypeptide is fused to additional components of SEQ ID NO:1, for example, some or all of the amino acids at positions 162-181 of SEQ ID NO:1. Therefore, in some embodiments, the CT polypeptide is fused to some or all of the amino acids that form the transmembrane domain of CTLA-4. Typically, the CT polypeptide is not fused to any component of the extracellular domain of CTLA-4.

An exemplary amino acid sequence for the CTLA-4 tail region of human CTLA-4 is 41 amino acids in length and has a sequence: AVSLSKMLKKRSPLTTGVYVKMPPTEPECEKQFQPYFIPIN (SEQ ID NG:3). The YVKM motif is underlined.

An exemplary nucleic acid sequence for the CTLA-4 tail region of human CTLA-4 is:

(SEQ ID NO: 4)
gctgtttctttgagcaaaatgctaaagaaaagaagccctcttacaacag
gggtctatgtgaaaatgcccccaacagagccagaatgtgaaaagcaatt
tcagccttattttattcccatcaat.

In some embodiments, the CT domain is, or is derived from, a human CTLA-4 homologue such as a paralogue or orthologue. CTLA-4 proteins from numerous other organisms are known in the art, and include, but are not limited to, those listed in Table 1, below, the UniProt database entry accession numbers and the sequences provided therein, all of which are specifically incorporated by reference herein in their entireties.

TABLE 1
Exemplary CTLA-4 proteins

| UniProtKB | | | | SEQ |
|---|---|---|---|---|
| Accession | Entry | | Amino Acid Sequence | ID |
| ID No: | name | Organism | of CTLA-4 tail | NO: |
| P09793 | CTLA4_ | Mus musculus | AVSLSKMLKKRSPLTTGVY | 89 |
| | MOUSE | (Mouse) | VKMPPTEPECEKQFQPYFI | |
| | | | PIN | |
| Q9XSI1 | CTLA4_ | Canis lupus | AVSLSKMLKKRSPLTTGVY | 90 |
| | CANLF | familiaris (Dog) | VKMPPTGPECEKQFQPYFI | |
| | | | PIN | |
| Q9MYX7 | CTLA4_ | Sus scrofa (Pig) | AVSLSKMLKKRSPLTTGVY | 91 |
| | PIG | | VKMPPTEPECEKQFQPYFI | |
| | | | PIN | |
| Q28090 | Q28090_ | Bos taurus | AVSLSKMLKKRSPLTTGVY | 92 |
| | BOVIN | (Bovine) | VKMPPTEPECEKQFQPYFI | |
| | | | PIN | |
| Q62859 | Q62859_ | Rattus norvegicus | AVSLNRTLKKRSPLTTGVY | 93 |
| | RAT | (Rat) | VKMPPTEPECEKQFQPYFI | |
| | | | PIN | |
| P42072 | CTLA4_ | Oryctolagus | AVSLSKMLKKRSPLTTGVY | 94 |
| | RABIT | cuniculus (Rabbit) | VKMPPTEPECEKQFQPYFI | |
| | | | PIN | |
| Q9GKP2 | Q9GKP2_ | Canis lupus | AVSLSKMLKKRSPLTTGVY | 95 |
| | CANLF | familiaris (Dog) | VKMPPTEPECEKQFQPYFI | |
| | | (Canis familiaris) | PIN | |
| A0A096MJE4 | A0A096MJE4_ | Rattus norvegicus | AVSLNRTLKKRSPLTTGVY | 96 |
| | RAT | (Rat) | VKMPPTEPECEKQFQPYFI | |
| | | | PIN | |
| O97631 | O97631_ | Ovis aries (Sheep) | AVSLSKMLKKRSPLTTGVY | 97 |
| | SHEEP | | VKMPPTEPECEKQFQPYFI | |
| | | | PIN | |
| Q9BDC4 | Q9BDC4_ | Macaca mulatta | AVSLSKMLKKRSPLTTGVY | 98 |
| | MACMU | (Rhesus macaque) | VKMPPTEPECEKQFQPYFI | |
| | | | PIN | |
| F6ZMI5 | F6ZMI5_ | Ornithorhynchus | MVALSKMIKKRSLLTTGVY | 99 |
| | ORNAN | anatinus (Duckbill | VKMPPPEPEHEKQFQPYFI | |
| | | platypus) | PIN | |
| Q6GTR6 | Q6GTR6_ | Mus musculus | AVSLSKMLKKRSPLTTGVY | 89 |
| | MOUSE | (Mouse) | VKMPPTEPECEKQFQPYFI | |
| | | | PIN | |
| F6WWE3 | F6WWE3_ | Equus caballus | AVSLSRMLKKRSPLTTGVY | 101 |
| | HORSE | (Horse) | VKMPPTEPECEKQFQPYFI | |
| | | | PIN | |
| Q9BDN7 | Q9BDN7_ | Papio anubis (Olive | AVSLSKMLKKRSPLTTGVY | 102 |
| | PAPAN | baboon) | VKMPPTEPECEKQFQPYFI | |
| | | | PIN | |
| G1R5Y1 | G1R5Y1_ | Nomascus | AVSLSKMLKKRSPLTTGVY | 103 |
| | NOMLE | leucogenys | VKMPPTEPECEKQFQPYFI | |
| | | (Northern white- | PIN | |
| | | cheeked gibbon) | | |
| | | (Hylobates | | |
| | | leucogenys) | | |
| G1PE26 | G1PE26_ | Myotis lucifugus | AVSLSKMLKKRSPLTTGVY | 104 |
| | MYOLU | (Little brown bat) | VKMPPTEPECEKQFQPYFI | |
| | | | PIN | |
| M3XMW9 | M3XMW9_ | Mustela putorius | AISLSKMLKKRSPLTTGVY | 105 |
| | MUSPF | furo (European | VKMPPTEPECEKQFQPYFI | |
| | | domestic ferret) | PIN | |
| | | (Mustela furo) | | |
| Q7JHJ0 | Q7JHJ0_ | Macaca nemestrina | AVSLSKMLKKRSPLTTGVY | 106 |
| | MACNE | (Pig-tailed | VKMPPTEPECEKQFQPYFI | |
| | | macaque) | PIN | |
| A0A2I3SUF9 | A0A2I3SUF9_ | Pan troglodytes | AVSLSKMLKKRSPLTTGVY | 124 |
| | PANTR | (Chimpanzee) | VKMPPTEPECEKQFQPYFI | |
| | | | PIN | |
| Q7JHJ2 | Q7JHJ2_ | Cercocebus atys | AVSLSKMLKKRSPLTTGVY | 107 |
| | CERAT | (Sooty mangabey) | VKMPPTEPECEKQFQPYFI | |
| | | (Cercocebus | PIN | |
| | | torquatus atys) | | |

### 3. CTLA-4 Tail Variants and Multimers

Multimers and variants of the CTLA-4 tail and CT polypeptides are provided. Typically, the multimers and variants impart the function of enhancing cell cycling via endosomal trafficking that is associated with the intact CTLA-4 tail.

In some embodiments, the CT polypeptide includes SEQ ID NO:3 alone, or a functional variant or fragment thereof. For example, in some embodiments, the CT domain of a fusion peptide includes a CT polypeptide which has less than 41 amino acids and includes a single YVKM motif. Therefore, in some embodiments, the CT domain of a fusion peptide includes a CT polypeptide having an amino acid sequence of any one of SEQ ID NOs 5, or 9-23. In particular embodiments, the CT domain of a fusion peptide includes a CT polypeptide having 25 amino acids. In a particular embodiment, the CT domain of a fusion peptide includes a CT polypeptide having an amino acid sequence of SEQ ID NO:5.

In other embodiments, the CT polypeptide includes two or more YVKM motifs. For example, in some embodiments, the CT polypeptide includes the entire amino acid sequence of SEQ ID NO:3, or a functional variant or fragment thereof, contiguous with one or more CT polypeptides incorporating one or more additional YVKM motifs. In some embodiments, the CT domain of a fusion peptide includes a first CT polypeptide which has less than 41 amino acids and includes a single YVKM motif, contiguous with one or more additional CT polypeptides incorporating one or more additional YVKM motifs. In particular embodiments, the CT domain of a fusion peptide includes a first CT polypeptide which has an amino acid sequence of SEQ ID NO:5, contiguous with one or more additional CT polypeptides incorporating one or more additional YVKM motifs.

In some embodiments, the CT domain of a fusion peptide includes a CT polypeptide having two YVKM motifs. In other embodiments, the CT domain of a fusion peptide includes a CT polypeptide having three YVKM motifs. Therefore, in some embodiments, the CT domain of a fusion peptide includes a CT polypeptide having a first amino acid sequence of SEQ ID NO:3, or a functional fragment or variant thereof, contiguous with a second amino acid sequence of SEQ ID NO:3, or a functional fragment or variant thereof. Exemplary fragments of SEQ ID NO:3 include the amino acid sequences of any one of SEQ ID NOs 5, or 9-23, as set forth in more detail below.

a. CT Multimers

In some embodiments, the CT domain within a fusion protein includes two or more CT polypeptides that are multiplexed, for example, by contiguous fusion of the two polypeptides together, to provide two or more copies of any functional component of the CTLA-4 tail. As described in the Example, the cycling function of the CT domain that can be imparted to fusion proteins is enhanced by multiplexing. It may be that the cycling function is associated with the YVKM motif in the CT domain. Therefore, multiplexing of CT domains preferably provides at least two copies of the YVKM motif.

In some embodiments, CT domains include two or more copies of all, or at least a portion of SEQ ID NO:3. In some embodiments, CT domains include at least SEQ ID NO:3 and one or more additional copies of SEQ ID NO:3, optionally, but preferably, configured to be immediately contiguous with the first copy of SEQ ID NO:3.

In other embodiments, a CT polypeptide multimer includes SEQ ID NO:3 together with one or more functional fragments or variants of SEQ ID NO:3, optionally, but preferably, configured to be immediately contiguous with SEQ ID NO:3. An exemplary CT polypeptide for including within a CT domain of a fusion peptide having a CT multimer is 25 amino acids in length, having an amino acid sequence: GVYVKMPPTEPECEKQFQPYFIPIN (SEQ ID NO:5). The YVKM amino acid motif is underlined.

An exemplary nucleic acid sequence of a CT polypeptide of SEQ ID NO:5 for including within a fusion peptide having a CT multimer is:

(SEQ ID NO: 6)
ggcgtctacgttaagatgccacccacggagccagaatgcgaaaaacagt
ttcaaccatatttcataccaataaac.

### Double CT (CT(2)) Polypeptides

In some embodiments, the CT domain of a fusion peptide includes a first polypeptide fused to, or contiguous with, a second CT polypeptide, having a first and second YVKM motifs (“double CT polypeptide”; “CT(2)”). In some embodiments, the double CT polypeptide includes two copies of SEQ ID NO:3. For example, in some embodiments, a CT2 polypeptide has the amino acid sequence: AVSLSKMLKKRSPLTTGVYVKMPPTEPECEKQFQPYFIP INAVSLSKMLKKRSPLTTGV YVKMPPTEPECEKQFQPYF IP IN (SEQ ID NO:88). The YVKM amino acid motifs are underlined.

In other embodiments, a CT(2) polypeptide includes one copy of SEQ ID NO:3 and one copy of SEQ ID NO:5 (i.e., CT(2)). Therefore, in some embodiments, the amino acid sequence of the CT(2) polypeptide is: AVSLSKMLKKRSPLTTGVYVKMPPTEPECEKQFQPYFIPINGVYVKMPPTEPECEKQFQ PYFIPIN (SEQ ID NO:7). The YVKM amino acid motifs are underlined.

Therefore, in some embodiments, the nucleic acid sequence encoding the CT(2) polypeptide of SEQ ID NO:7 is:

(SEQ ID NO: 8)
gctgtttctttgagcaaaatgctaaagaaaagaagccctcttacaacag
gggtctatgtgaaaatgcccccaacagagccagaatgtgaaaagcaatt
tcagccttattttattcccatcaatggcgtctacgttaagatgccaccc
acggagccagaatgcgaaaaacagtttcaaccatatttcataccaataa
ac.

### Triple CT (CT(3)) Polypeptides

In some embodiments, the CT domain includes a first and second and third YVKM motifs. For example, in some embodiments, the CT polypeptide includes both SEQ ID NO:3 and two copies of SEQ ID NO:5 (i.e., CT(3)). Therefore, in some embodiments, the amino acid sequence of the CT(3) polypeptide is:

(SEQ ID NO: 51)
AVSLSKMLKKRSPLTTGVYVKMPPTEPECEKQFQPYFIPINGVYVKMPP
TEPECEKQFQPYFIPINGVYVKMPPTEPECEKQFQPYFIPIN.

In some embodiments a nucleic acid sequence encoding the CT(3) polypeptide is:

(SEQ ID NO: 52)
gctgtttctttgagcaaaatgctaaagaaaagaagccctcttacaacag
gggtctatgtgaaaatgcccccaacagagccagaatgtgaaaagcaatt
tcagccttattttattcccatcaatggcgtctacgttaagatgccaccc
acggagccagaatgcgaaaaacagtttcaaccatatttcataccaataa
acggggtctatgttaaaatgccgcctactgagcctgagtgtgaaaaaca
attccagccatattttatacccataaat.

### Quadruple (CT(4)) and Further CT Polypeptides

In some embodiments, the CT domain includes four or more YVKM motifs. For example, in some embodiments, the CT polypeptide includes both SEQ ID NO:3 and three or more copies of SEQ ID NO:5 (i.e., CT(4)). Therefore, in some embodiments, the amino acid sequence of the CT(4) polypeptide is: AVSLSKMLKKRSPLTTGVYVKMPPTEPECEKQFQPYFIPINGVYVKMPPTEPECEKQFQ PYFIPINGVYVKMPPTEPECEKQFQPYFIPINGVYVKMPPTEPECEKQFQPYFIPIN (SEQ ID NO:53). The YVKM amino acid motifs are underlined.

In some embodiments a nucleic acid sequence encoding the CT(4) polypeptide is:

(SEQ ID NO: 54)
gctgtttctttgagcaaaatgctaaagaaaagaagccctcttacaacaggggtctatgt
gaaaatgcccccaacagagccagaatgtgaaaagcaatttcagccttattttattccca
tcaatggcgtctacgttaagatgccacccacggagccagaatgcgaaaaacagtttcaa
ccatatttcataccaataaacggggtctatgttaaaatgccgcctactgagcctgagtg
tgaaaaacaattccagccatattttatacccataaatggcgtctacgttaagatgccac
ccacggagccagaatgcgaaaaacagtttcaaccatatttcataccaataaac.

Further multimers of CT polypeptides, including more copies of the YVKM motif can be constructed, for example, by adding one or more further copies of SEQ ID NO:5 to the polypeptide of SEQ ID NO:53. Therefore, CT domains can include multimers of CT polypeptides, such as contiguous, “tandem” multimers. In some embodiments, CT domains include the polypeptide of SEQ ID NO:3 with the addition of 1 or 2, 3, 4, 5, 6, 7, 8, 9, or 10 or more than 10 contiguous copies of the polypeptide of SEQ ID NO:5, to form CT domains having 2, 3, 4, 5, 6, 7, 8, 9, 10, 11 or more than 11 YVKM motifs, respectively.

A preferred CT domain has 2 copies of YVKM motif, for example, as in SEQ ID NO: 7.

b. CT Variants

It has been discovered that the CT polypeptide is sufficient to drive enhanced endosomal cycling of a recombinant fusion protein including the CT polypeptide and one or more additional domains. Thus, compositions and methods of use of CT peptide and fusion peptides thereof are provided. The compositions typically are, or include, a CT polypeptide (SEQ ID NO:3, 5 or 7) or a functional fragment or variant thereof, or a nucleic acid encoding the same. Functional fragments and variants can be, for example, any number of amino acids sufficient to drive enhanced endosomal cycling. The data below supports the conclusions that CT enhances tumor cell killing by CAR-T cells having a CAR-CT fusion, by increased endosomal cycling through interaction between AP-2 and the YVKM motif in CT.

In some embodiments, the fragment is between about 10 amino acids and about 195 amino acids, inclusive of SEQ ID NO:1 or a homologue such as an orthologue or paralogue thereof; or any combination thereof, or any subrange thereof, or any specific integer number of amino acids therebetween, including, but not limited to 20, 25, 30, 35, 36, 39, 40, 41, 45, 50, 60, 66, 70, 75, 100, 125, 150, or 175 amino acids. Variants can have, for example, at least 65%, 70%, 75%, 80%, 85%, 90%, 95%, 99% or 100% sequence identity to SEQ ID NO:3, 5, or 7, or a functional fragment thereof; or the corresponding sequence of a homologue such as an orthologue or paralogue of any of the foregoing sequences; or any combination thereof. In a particular embodiment, a variant CT polypeptide has at least 75%, 80%, 85%, 90%, 95%, 99% or 100% sequence identity to SEQ ID NO:3. In a particular embodiment, a variant CT polypeptide has at least 75%, 80%, 85%, 90%, 95%, 99% or 100% sequence identity to SEQ ID NO:5. In a particular embodiment, a variant CT polypeptide has at least 75%, 80%, 85%, 90%, 95%, 99% or 100% sequence identity to SEQ ID NO:7. Preferably variants maintain the ability to interact with AP-2, i.e., maintain the YVKM motif in CT. In some embodiments, a CT polypeptide variant is considered to be “functional” if it maintains the ability to interact with AP-2, i.e., maintains the YVKM motif in CT. In some embodiments, CT polypeptide variants are identified as functional if they interact with AP-2 and/or co-immuno-precipitate AP-2. Exemplary variants of CT polypeptides include deletions of amino acids at either end of SEQ ID NO:3, such as:

(SEQ ID NO: 9)
VSLSKMLKKRSPLTTGVYVKMPPTEPECEKQFQPYFIPIN;
(SEQ ID NO: 10)
SLSKMLKKRSPLTTGVYVKMPPTEPECEKQFQPYFIPIN;
(SEQ ID NO: 11)
LSKMLKKRSPLTTGVYVKMPPTEPECEKQFQPYFIPIN;
(SEQ ID NO: 12)
SKMLKKRSPLTTGVYVKMPPTEPECEKQFQPYFIPIN;
(SEQ ID NO: 13)
KMLKKRSPLTTGVYVKMPPTEPECEKQFQPYFIPIN;
(SEQ ID NO: 14)
MLKKRSPLTTGVYVKMPPTEPECEKQFQPYFIPIN;
(SEQ ID NO: 15)
LKKRSPLTTGVYVKMPPTEPECEKQFQPYFIPIN;
(SEQ ID NO: 16)
KKRSPLTTGVYVKMPPTEPECEKQFQPYFIPIN;
(SEQ ID NO: 17)
KRSPLTTGVYVKMPPTEPECEKQFQPYFIPIN;
(SEQ ID NO: 18)
RSPLTTGVYVKMPPTEPECEKQFQPYFIPIN;
(SEQ ID NO: 19)
SPLTTGVYVKMPPTEPECEKQFQPYFIPIN;
(SEQ ID NO: 20)
PLTTGVYVKMPPTEPECEKQFQPYFIPIN;
(SEQ ID NO: 21)
LTTGVYVKMPPTEPECEKQFQPYFIPIN;
(SEQ ID NO: 22)
TTGVYVKMPPTEPECEKQFQPYFIPIN;
(SEQ ID NO: 23)
TGVYVKMPPTEPECEKQFQPYFIPIN;
(SEQ ID NO: 24)
GVYVKMPP;
(SEQ ID NO: 25)
VYVKMPPTEPECEKQFQPYFIPIN;
(SEQ ID NO: 26)
YVKMPPTEPECEKQFQPYFIPIN;
(SEQ ID NO: 27)
VSLSKMLKKRSPLTTGVYVKMPPTEPECEKQFQPYFIPI;
(SEQ ID NO: 28)
VSLSKMLKKRSPLTTGVYVKMPPTEPECEKQFQPYFIP;
(SEQ ID NO: 29)
VSLSKMLKKRSPLTTGVYVKMPPTEPECEKQFQPYFI;
(SEQ ID NO: 30)
VSLSKMLKKRSPLTTGVYVKMPPTEPECEKQFQPYF;
(SEQ ID NO: 31)
VSLSKMLKKRSPLTTGVYVKMPPTEPECEKQFQPY;
(SEQ ID NO: 32)
VSLSKMLKKRSPLTTGVYVKMPPTEPECEKQFQP;
(SEQ ID NO: 33)
VSLSKMLKKRSPLTTGVYVKMPPTEPECEKQFQ;
(SEQ ID NO: 34)
VSLSKMLKKRSPLTTGVYVKMPPTEPECEKQF;
(SEQ ID NO: 35)
VSLSKMLKKRSPLTTGVYVKMPPTEPECEKQ;
(SEQ ID NO: 36)
VSLSKMLKKRSPLTTGVYVKMPPTEPECEK;
(SEQ ID NO: 37)
VSLSKMLKKRSPLTTGVYVKMPPTEPECE;
(SEQ ID NO: 38)
VSLSKMLKKRSPLTTGVYVKMPPTEPEC;
(SEQ ID NO: 39)
VSLSKMLKKRSPLTTGVYVKMPPTEPE;
(SEQ ID NO: 40)
VSLSKMLKKRSPLTTGVYVKMPPTEP;
(SEQ ID NO: 41)
VSLSKMLKKRSPLTTGVYVKMPPTE;
(SEQ ID NO: 42)
VSLSKMLKKRSPLTTGVYVKMPPT;
(SEQ ID NO: 43)
VSLSKMLKKRSPLTTGVYVKMPP;
(SEQ ID NO: 44)
VSLSKMLKKRSPLTTGVYVKMP;
(SEQ ID NO: 45)
SLSKMLKKRSPLTTGVYVKMPPTEPECEKQFQPYFIPI;
(SEQ ID NO: 46)
LSKMLKKRSPLTTGVYVKMPPTEPECEKQFQPYFIPI;
(SEQ ID NO: 47)
SKMLKKRSPLTTGVYVKMPPTEPECEKQFQPYFIP;
(SEQ ID NO: 48)
KMLKKRSPLTTGVYVKMPPTEPECEKQFQPYFI;
(SEQ ID NO: 49)
MLKKRSPLTTGVYVKMPPTEPECEKQFQPYF;
(SEQ ID NO: 50)
LKKRSPLTTGVYVKMPPTEPECEKQFQPY;
(SEQ ID NO: 108)
GVYVKMPPTEPECEKQFQPYFIPI;
(SEQ ID NO: 109)
GVYVKMPPTEPECEKQFQPYFIP;
(SEQ ID NO: 110)
GVYVKMPPTEPECEKQFQPYFI;
(SEQ ID NO: 111)
GVYVKMPPTEPECEKQFQPYF;
(SEQ ID NO: 112)
GVYVKMPPTEPECEKQFQPY;
(SEQ ID NO: 113)
GVYVKMPPTEPECEKQFQP;
(SEQ ID NO: 114)
GVYVKMPPTEPECEKQFQ;
(SEQ ID NO: 115)
GVYVKMPPTEPECEKQF;
(SEQ ID NO: 116)
GVYVKMPPTEPECEKQ;
(SEQ ID NO: 117)
GVYVKMPPTEPECEK;
(SEQ ID NO: 118)
GVYVKMPPTEPECE;
(SEQ ID NO: 119)
GVYVKMPPTEPEC;
(SEQ ID NO: 120)
GVYVKMPPTEPE;
(SEQ ID NO: 121)
GVYVKMPPTEP;
(SEQ ID NO: 122)
GVYVKMPPTE;
(SEQ ID NO: 123)
GVYVKMPPT.
The YVKM motifs are underlined.

Any one or more of SEQ ID NOS:3, 5, 9-50, 108-123 or a homologue or variant thereof can be used as a CT domain, either as a single CT polypeptide, or in combination with SEQ ID NO:3 as double or multiplexed tandem CT polypeptide. For example, a CT domain may include any one of SEQ ID NOs:3, 5 or 9-50, or 108-123 multiplexed with one or more of SEQ ID NOS:3, 5, or 9-50, or 108-123. In an exemplary embodiment, a double CT (CT(2)) polypeptide includes SEQ ID NO:3, and any one of SEQ ID NOS:9-50. For example, in one embodiment, a double CT (CT(2)) polypeptide includes SEQ ID NO:3, and SEQ ID NO:25. In another exemplary embodiment, a triple CT (CT(3)) polypeptide includes SEQ ID NO:7, and any one of SEQ ID NOS:9-50. In an exemplary embodiment, a triple CT (CT(3)) polypeptide includes SEQ ID NO:7, and SEQ ID NO:25. In other embodiments, a triple CT (CT(3)) polypeptide includes SEQ ID NO:3, and any one of SEQ ID NOS:9-50 and SEQ ID NO:25. In some embodiments, any one or more of SEQ ID NOS:3, 9, 10, 11, or 12, or a functional homologue or variant thereof is a CT polypeptide that forms a CT domain of a CT fusion peptide. In other embodiments, any one or more of SEQ ID NOS:3, 9, 10, 11, or 12, or a functional homologue or variant thereof is a CT polypeptide that forms part of a multiplexed CT domain in combination with another functional CT polypeptide, for example, by fusion directly with the second CT polypeptide. In some embodiments, the functional CT domain of a second or further CT polypeptide is the amino acid sequence SEQ ID NO:5. Therefore, in some embodiments, a double or multiplexed tandem CT polypeptide includes the amino acid sequence of any one of SEQ ID NOS:3, 9, 10, 11, or 12, or a functional homologue or variant thereof, contiguous with SEQ ID NO:5. For example, in particular embodiments, a double CT (CT(2)) polypeptide includes SEQ ID NO:10 contiguous with SEQ ID NO:5. In other embodiments, a double CT (CT(2)) polypeptide includes SEQ ID NO:9 contiguous with SEQ ID NO:5. In other embodiments, a double CT (CT(2)) polypeptide includes SEQ ID NO:12 contiguous with SEQ ID NO:5. In other embodiments, a double CT (CT(2)) polypeptide includes SEQ ID NO:11 contiguous with SEQ ID NO:5. In some embodiments, a multiplexed tandem CT polypeptide includes the amino acid sequence of any one of SEQ ID NOS:3, 9, 10, 11, or 12, or a homologue or variant thereof, contiguous with two or more copies of SEQ ID NO:5. For example, in some embodiments, a triple CT (CT(3)) polypeptide includes SEQ ID NO:10 contiguous with two copies of SEQ ID NO:5. In other embodiments, a double CT (CT(2)) polypeptide includes SEQ ID NO:9 contiguous with two copies of SEQ ID NO:5. In other embodiments, a double CT (CT(2)) polypeptide includes SEQ ID NO:12 contiguous with two copies of SEQ ID NO:5. In other embodiments, a double CT (CT(2)) polypeptide includes SEQ ID NO:11 contiguous with two copies of SEQ ID NO:5.

Any of the CT polypeptide sequences including the amino acid sequences of SEQ ID NOs:3, 5, 7, 9-51 or 108-123 can include one or more amino acid substitutions. Typically, the amino acid substitutions do not alter the YVKM motif, or do not impact the ability of the CT domain of a fusion peptide to interact with the AP-2 protein. Thus, in preferred embodiments, the CT polypeptides include at least one YVKM motif. Amino acid substitutions within CT peptides are generally based on the relative similarity of the amino acid side-chain substituents, for example, their hydrophobicity, hydrophilicity, charge, size, and the like. Exemplary substitutions of amino acids within any of SEQ ID NOs:3, 5, 7, or 9-50, or 108-123 can include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a CT polypeptide, as set forth in any one of SEQ ID NOs:3, 5, 7, or 9-50 or 108-123. In particular embodiments, the CT polypeptides can include variants having about 50%, 60%, 70%, 80%, 90%, and 95% sequence identity to the polypeptide of SEQ ID NOs:3, 5, 7, or 9-50. Such variants and fragments can be used alone or in combination with each other and/or SEQ ID NOs:3, 5, 7, or 9-50, or 108-123 or homologues thereof as described above, e.g., for SEQ ID NOs:3, 5, 7, or 9-50, or 108-123.

### B. CT Fusion Peptides

CT-fusion proteins, including one or more heterologous polypeptide sequences fused to one or more CT polypeptides are provided.

The term “CT domain” is used exclusively in the context of fusion peptides that include one or more CT polypeptides, to refer to the component of the fusion peptide that includes the CT polypeptide(s). In some embodiments, the CT domain of a fusion peptide includes all of the amino acids that form the intracellular CTLA-4 tail. Therefore, in some embodiments, a CT domain includes all of the amino acids at positions 182-223 of SEQ ID NO:1. Therefore, in some embodiments, the CT domain includes all of SEQ ID NO:3. In some embodiments, the CT domain includes all of SEQ ID NO:7. In some embodiments, the CT domain includes all of SEQ ID NO:51. In some embodiments, the CT domain includes all or part of SEQ ID NO:5. In some embodiments, the CT domain includes any of SEQ ID NOs:9-50.

The term “CT fusion protein” or “CT fusion peptide” or “CT fusion” refers to a polypeptide that includes a CT domain and one or more heterologous amino acid sequences. A heterologous sequence for inclusion within a CT fusion protein is not a component of a CTLA-4 peptide.

The term “fusion peptide domain” refers to a heterologous sequence that is directly or indirectly fused to a CT domain. Typically a fusion peptide domain includes at least one amino acid that is on the extracellular side of the cell membrane, i.e., constitutes an extracellular domain. In some embodiments, the fusion peptide domain also includes a transmembrane domain.

Since the CT polypeptides are derived from the cytosolic component of the CTLA-4 molecule, CT-fusion proteins are typically engineered to include one or more CT domains as a cytosolic component of the CT fusion protein. Therefore, in preferred embodiments, CT fusion peptides include one or more heterologous polypeptides fused to an intracellular component, including a CT domain that includes SEQ ID NO:3, or SEQ ID NO:7, or SEQ ID NO:51, or SEQ ID NO:5, or any of SEQ ID NOs:9-50, or 108-123, or a functional homologue or variant thereof. An exemplary heterologous polypeptide for inclusion within CT-fusion peptides includes the ectodomain and transmembrane domain of a cell-surface protein, such as an ectodomain of a cell-surface receptor that is not a component of CTLA-4.

In some embodiments, CT-fusion proteins include one or more extracellular polypeptide domains, and one or more transmembrane domains, and optionally an intracellular domain, where the transmembrane domain or optionally the intracellular domain is fused to the CT domain(s). In some embodiments, where a fusion protein includes one or more intracellular domains, the one or more CT domains are fused to the amino (N) or carboxyl (C) terminus of the intracellular domains. In some embodiments, the fusion proteins include an entire endogenous protein fused to one or more CT domains. Exemplary schematics for the domain structure of a fusion protein include:
N-[Fusion peptide domain(s)]-[CT domain]-C; or
N-[CT domain]-[Fusion peptide domain(s)]-C; or
N-[Fusion peptide domain(s)]-[CT domain]-[Fusion peptide domain(s)]-C,
where “N” and “C” refer to the amino (NH2) and Carboxyl (COOH) termini, respectively.

The number of functional domains and CT domains can vary according to the requirements of the fusion peptide. For example, the domain structure of a fusion protein can include:
N-[Fusion peptide domain]_{X}-[CT domain]_{Y}-C; or
N-[CT domain]_{X}-[Fusion peptide domain]_{Y}-C; or
N-[Fusion peptide domain]_{X}-[CT domain]_{Y}-[Fusion peptide domain]_{Z}-C,
Where X, Y and Z are independently integers of between 1 and 10 and “N” and “C” refer to the ammino (NH2) and Carboxyl (COOH) termini, respectively.

In preferred embodiments, Y is 1, 2 or 3.

In some embodiments, the fusion proteins are configured according to the orientation of the different domains relative to the cellular membrane. For example, fusion peptides can be configured to include extracellular, transmembrane and intracellular components. Therefore, in some embodiments, the one or more fusion domains include one or more of an extracellular domain, a transmembrane domain and optionally an intracellular domain (i.e., further intracellular domain in addition to the CT polypeptide). In other embodiments, the one or more fusion domains include a trans-membrane domain and optionally an intracellular domain. In other embodiments the one or more functional domains include a first transmembrane domain, one or more extracellular domain(s), a second trans-membrane domain and optionally an intracellular domain. In some embodiments a fusion domain includes only a trans-membrane domain, or only an intracellular domain. Exemplary schematics for the domain structure of a fusion protein relative to the cell surface include:
N-[extracellular]_{X}-[transmembrane]-[CT]_{Y}-C; or
N-[extracellular]_{X}-[transmembrane]-[CT]_{Y}-[intracellular]_{Z}-C; or
N-[extracellular]_{X}-[transmembrane]-[intracellular]_{Z}-[CT]_{Y}-[intracellular]_{W}-C; or
N-[transmembrane]-[CT]_{Y}-[intracellular]_{Z}-C; or
N-[transmembrane]-[intracellular]_{Z}-[CT]_{Y}-[intracellular]_{W}-C,
Where X, Y, Z and W are independently integers of between 1 and 10, and “N” and “C” refer to the ammino (NH2) and Carboxyl (COOH) termini, respectively.

In preferred embodiments, Y is 1, 2 or 3.

In some embodiments, “CT” is any one or more of SEQ ID NOs:3, 5, 7, 9-51, or 108-123.

Extracellular, transmembrane and intracellular protein domains are known in the art and can be appended to the CT domains as designed by the requirements of the fusion peptide.

### 1. Heterologous Sequences

Heterologous elements that can be associated with, linked, conjugated, or otherwise attached directly or indirectly to the CT polypeptide sequence(s), or nucleic acids expressing the CT polypeptides are disclosed. Such molecules include, but are not limited to, protein domains, such as transduction domains, fusogenic peptides, targeting molecules, and sequences that enhance protein expression and/or isolation.Suitable protein domains include ectodomains, transmembrane domains, cytoplasmic domains of proteins and macromolecular structures including combinations of ectodomains, transmembrane domains, and cytoplasmic domains. Typically the other protein domains are not proteins from a CTLA-4 protein. In some embodiments, the other protein domains have or have potential for one or more molecular functions or activities. Such “functional” domains can be engineered to provide one or more functions or activities, as desired. Exemplary functions include receptor or ligand binding, enzymic activity, and molecular transport, such as active transport of one or more molecules into or out of one or more cellular compartments. In some embodiments, the other protein domains within a CT fusion protein bind to a specific substrate or molecule. An exemplary molecule is an antigen or a cell-surface receptor.

Thus, in some embodiments, CT fusion peptides include one or more heterologous peptide domains, such as receptors at the surface of a cell, optionally including a transmembrane domain that anchors or connects the ectodomain to the cell surface and connects with the intracellular CT domain having one or more YVKM motif(s).

Exemplary cell surface receptors coordinate the activity of cells upon interaction with other cells, such as immune cells, such as T cells. For example, in some embodiments, the heterologous domain is a recombinant or engineered chimeric antigen receptor (CAR). In other embodiments, the heterologous domain is a Programmed death protein 1 (PD1) domain. In some embodiments, the fusion peptides include multiple heterologous domains, such as a CAR or PD1 domain, as well as a T2A sequence that enhances cell expression, and one or more leader sequences, such as a CD8 leader sequence.

a. Chimeric Antigen Receptors (CAR)

In some forms, the fusion protein includes a Chimeric Antigen Receptor (CAR) fused with one or more CT domains (“CAR-CT”). Typically, CARs include a transmembrane domain and one or more intracellular/cytoplasmic domains. Therefore, in some embodiments, a CAR-CT fusion protein includes an entire CAR fused to one or more CT domains at the C terminus. In some embodiments, the CAR includes an intracellular domain that includes a component of endogenous CD3 zeta protein. Therefore, in some embodiments, a CAR-CT fusion protein includes an entire CAR, with one or more CT domains fused directly to the end of the CD3 zeta region, such that both the CD3 zeta and contiguous CT domain(s) are expressed in the cytoplasmic compartment.

In some embodiments, the addition of a CT polypeptide alters the dynamics of CAR molecules by one or more of accelerating CAR endocytosis, degradation, and/or recycling, reduced CAR-T tonic signaling and/or dampened T cell activation and/or inflammatory cytokine production, reduction in trogocytosis and/or cancer antigen loss, reduced potential for CAR-T fratricide, improved survival and/or persistence, enhanced anti-cancer functionality (e.g., upon repeated cancer stimulation), increased in vivo persistence, and/or enrichment for Tcm differentiation (e.g., relative to a control without the CT polypeptide).

### CAR Structure

CARs are engineered receptors that possess both antigen-binding and T-cell-activating functions. Immunotherapy using T cells genetically engineered to express a CAR is rapidly emerging as a promising new treatment for hematological and non-hematological malignancies. Based on the location of the CAR in the membrane of the cell, the CAR can be divided into three main distinct domains, including an extracellular antigen-binding domain, followed by a space region, a transmembrane domain, and the intracellular signaling domain. The antigen-binding domain, most commonly derived from variable regions of immunoglobulins, typically contains VH and VL chains that are joined up by a linker to form the so-called “scFv.” The segment interposing between the antigen-binding domain (e.g., scFv) and the transmembrane domain is a “spacer domain.” The spacer domain can include the constant IgG1 hinge-CH2-CH3 Fc domain. In some cases, the spacer domain and the transmembrane domain are derived from CD8. The intracellular signaling domains mediating T cell activation can include a CD3ƒ co-receptor signaling domain derived from C-region of the TCR α and β chains and one or more costimulatory domains. In some forms, conjugation with one or more CT domains includes addition of the one or more CT domains immediately next to the costimulatory domains of the CAR.

In some forms, the antigen-binding domain is derived from an antibody. The term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen-binding domain of a CAR can contain complementary determining regions (CDR) of an antibody, variable regions of an antibody, and/or antigen binding fragments thereof. For example, the antigen-binding domain for a CD19 CAR can be derived from a human monoclonal antibody to CD19, such as those described in U.S. Pat. No. 7,109,304, which is specifically incorporated by reference herein in its entirety for use in accordance with the disclosed compositions and methods. In some forms, the antigen-binding domain can include an F(ab′)2, Fab′, Fab, Fv or scFv.

In some forms, the CAR includes one or more spacer domain(s) (also referred to as hinge domain) that is located between the extracellular antigen-binding domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (GxS)n linker, wherein x and n, independently can be an integer of 3 or more, including 3, 4, 5, 6, 7, 8, 9, 10, 11, 12, or more.

In some forms, the CAR includes a transmembrane domain that can be directly or indirectly fused to the antigen-binding domain. The transmembrane domain may be derived either from a natural or a synthetic source. In some forms, the transmembrane domain of the CAR includes a transmembrane domain of an alpha, beta or zeta chain of a T-cell receptor, CD8, CD4, CD28, CD137, CD80, CD86, CD152 (CTLA-4) or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

The intracellular signaling domain is responsible for activation of at least one of the normal effector functions of the immune effector cell expressing the CAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma or epsilon), MB1 chain, B29, Fc RIII, Fc RI and combinations of signaling molecules such as CD3ƒ and CD28, 4-1BB, OX40 and combination thereof, as well as other similar molecules and fragments. Intracellular signaling portions of other members of the families of activating proteins can be used, such as FcγRIII and FcεRI.

Many immune effector cells require co-stimulation, in addition to stimulation of an antigen-specific signal, to promote cell proliferation, differentiation and survival, as well as to activate effector functions of the cell. Therefore, in some forms, the CAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from CD27, CD28, CD137, 0X40, CD30, CD40, CD3, LFA-1, ICOS, CD2, CD7, LIGHT, NKG2C, B7-H3, ligands of CD83 and combinations thereof.

CARs can be used in order to generate immuno-responsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO 9215322, each of which is specifically incorporated by reference herein in its entirety). Alternative CAR constructs can be characterized as belonging to successive generations. First-generation CARs typically include a single-chain variable fragment of an antibody specific for an antigen, for example including a VL linked to a VH of a specific antibody, linked by a flexible linker, for example by a CD8α hinge domain and a CD8α transmembrane domain, to the transmembrane and intracellular signaling domains of either CD3ƒ or FcRγ (scFv-CD3ƒ or scFv-FcRγ; see U.S. Pat. Nos. 7,741,465; 5,912,172; 5,906,936, each of which is specifically incorporated by reference herein in its entirety). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ƒ; see U.S. Pat. Nos. 8,911,993; 8,916,381; 8,975,071; 9,101,584; 9,102,760; 9,102,761, each of which is specifically incorporated by reference herein in its entirety). Third-generation CARs include a combination of costimulatory endodomains, such a CD3ƒ-chain, CD97, GDI 1a-CD18, CD2, ICOS, CD27, CD154, CDS, OX40, 4-1BB, or CD28 signaling domains (for example scFv-CD28-4-1BB-CD3ƒ or scFv-CD28-OX40-CD3ƒ; see U.S. Pat. Nos. 8,906,682; 8,399,645; 5,686,281; PCT Publication No. WO2014134165; PCT Publication No. WO2012079000, each of which is specifically incorporated by reference herein in its entirety). Alternatively, co-stimulation can be orchestrated by expressing CARs in antigen-specific T cells, chosen so as to be activated and expanded following engagement of their native αβTCR, for example by antigen on professional antigen-presenting cells, with attendant co-stimulation. Any of the first, second, or third generation CARs described above can be used in accordance with the disclosed compositions and methods.

In some forms, the gene of interest within a transposon encodes a CAR targeting one or more antigens specific for cancer, an inflammatory disease, a neuronal disorder, HIV/AIDS, diabetes, a cardiovascular disease, an infectious disease, an autoimmune disease, or combinations thereof. One of skill in the art, based on general knowledge in the field and/or routine experimentation would be able to determine the appropriate antigen to be targeted by a CAR for a specific disease, disorder or condition.

Exemplary antigens specific for cancer that could be targeted by the CAR include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In preferred forms, the CAR targets CD19, CD22, or both CD19 and CD22.

Exemplary antigens specific for an inflammatory disease that could be targeted by the CAR include, but are not limited to, AOC3 (VAP-1), CAM-3001, CCL11 (eotaxin-1), CD 125, CD 147 (basigin), CD 154 (CD40L), CD2, CD20, CD23 (IgE receptor), CD25 (a chain of IL-2 receptor), CD3, CD4, CD5, IFN-a, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, LFA-1 (CD 11a), MEDI-528, myostatin, OX-40, rhuMAb β7, scleroscin, SOST, TGF beta 1, TNF-a, VEGF-A, and combinations thereof.

Exemplary antigens specific for a neuronal disorder that could be targeted by the CAR include, but are not limited to, beta amyloid, MABT5102A, and combinations thereof.

Exemplary antigens specific for diabetes that could be targeted by the CAR include, but are not limited to, L-I β, CD3, and combinations thereof.

Exemplary antigens specific for a cardiovascular disease that could be targeted by the CAR include, but are not limited to, C5, cardiac myosin, CD41 (integrin alpha-lib), fibrin II, beta chain, ITGB2 (CD 18), sphingosine-1-phosphate, and combinations thereof.

Exemplary antigens specific for an infectious disease that could be targeted by the CAR include, but are not limited to, anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNF-a, and combinations thereof.

In some forms, the CAR targets one or more antigens selected from an antigen listed in Table 2. In preferred forms, the CAR fused to CT includes one CAR ectodomain and a CT domain including two YVKM motifs, for example, as set forth in SEQ ID NO:7.

TABLE 2
Non-limiting examples of CAR targets

| | | | | | |
|---|---|---|---|---|---|
| AFP | AKAP-4 | ALK | Androgen receptor | B7H3 | BCMA |
| Bcr-Abl | BORIS | Carbonic | CD123 | CD138 | CD174 |
| CD19 | CD20 | CD22 | CD30 | CD33 | CD38 |
| CD80 | CD86 | CEA | CEACAM5 | CEACAM6 | Cyclin |
| CYP1B1 | EBV | EGFR | EGFR806 | EGFRvIII | EpCAM |
| EpCAM | EphA2 | ERG | ETV6-AML | FAP | Fos-related antigen1 |
| Fucosyl | fusion | GD2 | GD3 | GloboH | GM3 |
| gp100 | GPC3 | HER-2/neu | HER2 | HMWMAA | HPV E6/E7 |
| hTERT | Idiotype | IL12 | IL13RA2 | IM19 | IX |
| LCK | Legumain | lgK | LMP2 | MAD-CT-1 | MAD-CT-2 |
| MAGE | MelanA/MART1 | Mesothelin | MET | ML-IAP | MUC1 |
| Mutant p53 | MYCN | NA17 | NKG2D-L | NY-BR-1 | NY-ESO-1 |
| NY-ESO-1 | OY-TES1 | p53 | Page4 | PAP | PAX3 |
| PAX5 | PD-L1 | PDGFR-β | PLAC1 | Polysialic acid | Proteinase3 (PR1) |
| PSA | PSCA | PSMA | Ras mutant | RGS5 | RhoC |
| ROR1 | SART3 | sLe(a) | Sperm protein 17 | SSX2 | STn |
| Survivin | Tie2 | Tn | TRP-2 | Tyrosinase | VEGFR2 |
| WT1 | XAGE | Claudin6 | Claudin18.2 | CD70 | |

i. Structure of Chimeric Antigen Receptors with CT Domains

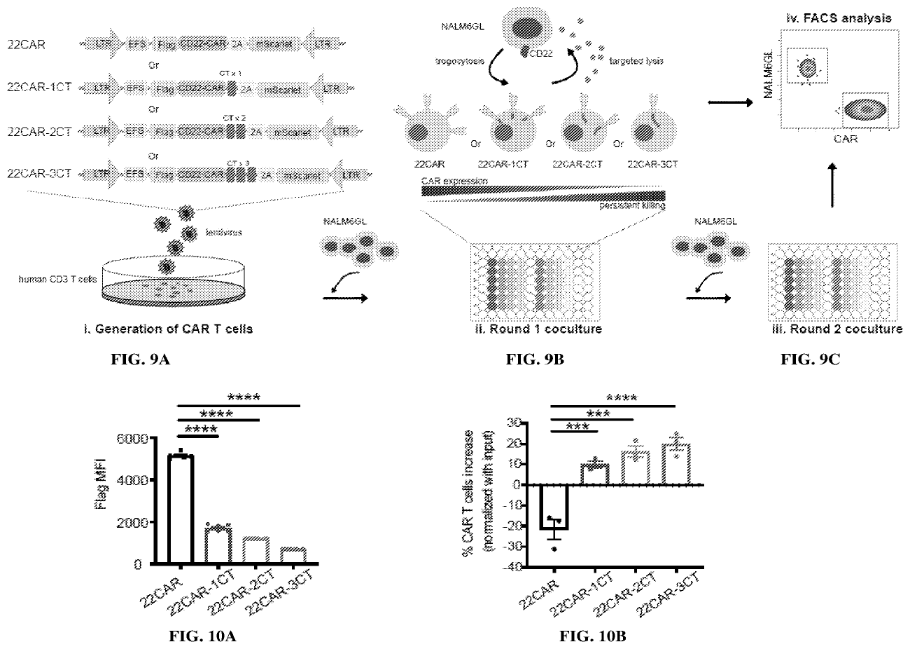

In some forms, the fusion peptide is a CAR including CT polypeptides having one or more YVKM motifs conjugated to the carboxyl terminus of the CAR. An exemplary schematic for conjugation of a functional CAR with CT domains is provided in FIGS. 9A and 12A.

In exemplary embodiments, the structure of a CAR-CT is fusion protein is:
N-[CAR]-[CT]_{Z}-C, where “N” and “C” refer to the anmmino (NH2) and Carboxyl (COOH) termini, respectively, and Z is an integer between one and four, inclusive. Preferably, Z is 1 or 2.

In some embodiments, a CAR-CT including one CT domain fused to the cytoplasmic domain of the functional CAR includes the amino acid sequence of SEQ ID NO:3 fused to the last residue of the CAR. In some embodiments, a CAR-CT including a CT domain having two YVKM motifs fused to the cytoplasmic domain of the functional CAR includes the amino acid sequence of SEQ ID NO:7 fused to the last residue of the CAR. In some embodiments, a CAR-CT including three CT domains fused to the cytoplasmic domain of the functional CAR includes the amino acid sequence of SEQ ID NO:51 fused to the last residue of the CAR.

ii. Exemplary Chimeric Antigen Receptors Including CT Domains (CAR-CT)

In some forms, the fusion peptide is a CAR including one or more CT domains. An exemplary CAR-CT is an anti-CD22 CAR-CT.

In some embodiments, an anti-CD22 CAR-CT including a CT domain having one YVKM motif is CD22(m971)-CAR-CT, having an amino acid sequence:

(SEQ ID NO: 55)
QVQLQQSGPGLVKPSQTLSLTCAISGDSVSSNSAAWNWIRQSPSRGLEWLGRTYYRSKW
YNDYAVSVKSRITINPDTSKNQFSLQLNSVTPEDTAVYYCAREVTGDLEDAFDIWGQGT
MVTVSSGGGGSDIQMTQSPSSLSASVGDRVTITCRASQTIWSYLNWYQQRPGKAPNLLI
YAASSLQSGVPSRFSGRGSGTDFTLTISSLQAEDFATYYCQQSYSIPQTFGQGTKLEIK
AAAGTTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPRAVSLSKML
KKRSPLTTGVYVKMPPTEPECEKQFQPYFIPIN.

The YVKM motif is underlined.

In some embodiments, an anti-CD22 CAR-CT including a CT domain having one YVKM motif is CD22(m971)-CAR-CT, having an amino acid sequence:

(SEQ ID NO: 56)
caggtgcagctgcagcagtctggccctggcctcgtgaagcctagccagaccctgagcct
gacctgtgccatcagcggcgatagcgtgtccagcaatagcgccgcctggaactggatca
gacagagccctagcagaggcctggaatggctgggccggacctactaccggtccaagtgg
tacaacgactacgccgtgtccgtgaagtcccggatcaccatcaaccccgacaccagcaa
gaaccagttctccctgcagctgaacagcgtgacccccgaggataccgccgtgtactact
gcgccagagaagtgaccggcgacctggaagatgccttcgacatctggggccagggcaca
atggtcaccgtgtctagcggaggcggcggaagcgacatccagatgacacagagccccag
ctccctgagcgccagcgtgggagacagagtgaccatcacctgtcgggccagccagacca
tctggtcctacctgaactggtatcagcagcggcctggcaaggcccccaacctgctgatc
tatgccgccagctcactgcagagcggcgtgcccagcagattttccggcagaggcagcgg
caccgacttcaccctgacaatcagttccctgcaggccgaggacttcgccacctactact
gccagcagagctacagcatcccccagaccttcggccaggggaccaagctggaaatcaaa
gcggccgcaggtaccaccacgacgccagcgccgcgaccaccaacaccggcgcccaccat
cgcgtcgcagcccctgtccctgcgcccagaggcatgccggccagcagcagggggcgcag
tgcacacgagggggctggacttcgcctgtgatatctacatctgggcgcccttggccggg
acttgtggggtccttctcctgtcactggttatcaccctttactgcaaacggggcagaaa
gaaactcctgtatatattcaaacaaccatttatgagaccagtacaaactactcaagagg
aagatggctgtagctgccgatttccagaagaagaagaaggaggatgtgaactgagagtg
aagttcagcaggagcgcagacgcccccgcgtaccagcagggccagaaccagctctataa
cgagctcaatctaggacgaagagaggagtacgatgttttggacaagagacgtggccggg
accctgagatggggggaaagccgagaaggaagaaccctcaggaaggcctgtacaatgaa
ctgcagaaagataagatggcggaggcctacagtgagattgggatgaaaggcgagcgccg
gaggggcaaggggcacgatggcctttaccagggtctcagtacagccaccaaggacacct
acgacgcccttcacatgcaggccctgccccctcgcgctgtttctttgagcaaaatgcta
aagaaaagaagccctcttacaacaggggtctatgtgaaaatgcccccaacagagccaga
atgtgaaaagcaatttcagccttattttattcccatcaat.

In some embodiments, an anti-CD22 CAR-CT(2) including a CT domain having two YVKM motifs is CD22(m971)-CAR-CT(2), having an amino acid sequence:

(SEQ ID NO: 57)
QVQLQQSGPGLVKPSQTLSLTCAISGDSVSSNSAAWNWIRQSPSRGLEWLGRTYYRSKW
YNDYAVSVKSRITINPDTSKNQFSLQLNSVTPEDTAVYYCAREVTGDLEDAFDIWGQGT
MVTVSSGGGGSDIQMTQSPSSLSASVGDRVTITCRASQTIWSYLNWYQQRPGKAPNLLI
YAASSLQSGVPSRFSGRGSGTDFTLTISSLQAEDFATYYCQQSYSIPQTFGQGTKLEIK
AAAGTTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPRAVSLSKML
KKRSPLTTGVYVKMPPTEPECEKQFQPYFIPINGVYVKMPPTEPECEKQFQPYFIPIN.

The YVKM motifs are underlined.

In some embodiments, an anti-CD22 CAR-CT(2) including two CT domains is CD22(m971)-CAR-CT(2), having a nucleic acid sequence::

(SEQ ID NO: 58)
caggtgcagctgcagcagtctggccctggcctcgtgaagcctagccagaccctgagcct
gacctgtgccatcagcggcgatagcgtgtccagcaatagcgccgcctggaactggatca
gacagagccctagcagaggcctggaatggctgggccggacctactaccggtccaagtgg
tacaacgactacgccgtgtccgtgaagtcccggatcaccatcaaccccgacaccagcaa
gaaccagttctccctgcagctgaacagcgtgacccccgaggataccgccgtgtactact
gcgccagagaagtgaccggcgacctggaagatgccttcgacatctggggccagggcaca
atggtcaccgtgtctagcggaggcggcggaagcgacatccagatgacacagagccccag
ctccctgagcgccagcgtgggagacagagtgaccatcacctgtcgggccagccagacca
tctggtcctacctgaactggtatcagcagcggcctggcaaggcccccaacctgctgatc
tatgccgccagctcactgcagagcggcgtgcccagcagattttccggcagaggcagcgg
caccgacttcaccctgacaatcagttccctgcaggccgaggacttcgccacctactact
gccagcagagctacagcatcccccagaccttcggccaggggaccaagctggaaatcaaa
gcggccgcaggtaccaccacgacgccagcgccgcgaccaccaacaccggcgcccaccat
cgcgtcgcagcccctgtccctgcgcccagaggcatgccggccagcagcagggggcgcag
tgcacacgagggggctggacttcgcctgtgatatctacatctgggcgcccttggccggg
acttgtggggtccttctcctgtcactggttatcaccctttactgcaaacggggcagaaa
gaaactcctgtatatattcaaacaaccatttatgagaccagtacaaactactcaagagg
aagatggctgtagctgccgatttccagaagaagaagaaggaggatgtgaactgagagtg
aagttcagcaggagcgcagacgcccccgcgtaccagcagggccagaaccagctctataa
cgagctcaatctaggacgaagagaggagtacgatgttttggacaagagacgtggccggg
accctgagatggggggaaagccgagaaggaagaaccctcaggaaggcctgtacaatgaa
ctgcagaaagataagatggcggaggcctacagtgagattgggatgaaaggcgagcgccg
gaggggcaaggggcacgatggcctttaccagggtctcagtacagccaccaaggacacct
acgacgcccttcacatgcaggccctgccccctcgcgctgtttctttgagcaaaatgcta
aagaaaagaagccctcttacaacaggggtctatgtgaaaatgcccccaacagagccaga
atgtgaaaagcaatttcagccttattttattcccatcaatggcgtctacgttaagatgc
cacccacggagccagaatgcgaaaaacagtttcaaccatatttcataccaataaac.

In some embodiments, an anti-CD22 CAR-CT(3) including three CT domains is CD22(m971)-CAR-CT(3), having an amino acid sequence:

(SEQ ID NO: 59)
QVQLQQSGPGLVKPSQTLSLTCAISGDSVSSNSAAWNWIRQSPSRGLEWLGRTYYRSKW
YNDYAVSVKSRITINPDTSKNQFSLQLNSVTPEDTAVYYCAREVTGDLEDAFDIWGQGT
MVTVSSGGGGSDIQMTQSPSSLSASVGDRVTITCRASQTIWSYLNWYQQRPGKAPNLLI
YAASSLQSGVPSRFSGRGSGTDFTLTISSLQAEDFATYYCQQSYSIPQTFGQGTKLEIK
AAAGTTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPRAVSLSKML
KKRSPLTTGVYVKMPPTEPECEKQFQPYFIPINGVYVKMPPTEPECEKQFQPYFIPING
VYVKMPPTEPECEKQFQPYFIPINCTEGRGSLLTCGDVEENPGP.

The YVKM motif is underlined.

In some embodiments, an anti CD22 CAR-CT(3) including a CT domain having two YVKM motifs is CD22(m971)-CAR-CT(3), having a nucleic acid sequence:

(SEQ ID NO: 60)
caggtgcagctgcagcagtctggccctggcctcgtgaagcctagccagaccctgagcct
gacctgtgccatcagcggcgatagcgtgtccagcaatagcgccgcctggaactggatca
gacagagccctagcagaggcctggaatggctgggccggacctactaccggtccaagtgg
tacaacgactacgccgtgtccgtgaagtcccggatcaccatcaaccccgacaccagcaa
gaaccagttctccctgcagctgaacagcgtgacccccgaggataccgccgtgtactact
gcgccagagaagtgaccggcgacctggaagatgccttcgacatctggggccagggcaca
atggtcaccgtgtctagcggaggcggcggaagcgacatccagatgacacagagccccag
ctccctgagcgccagcgtgggagacagagtgaccatcacctgtcgggccagccagacca
tctggtcctacctgaactggtatcagcagcggcctggcaaggcccccaacctgctgatc
tatgccgccagctcactgcagagcggcgtgcccagcagattttccggcagaggcagcgg
caccgacttcaccctgacaatcagttccctgcaggccgaggacttcgccacctactact
gccagcagagctacagcatcccccagaccttcggccaggggaccaagctggaaatcaaa
gcggccgcaggtaccaccacgacgccagcgccgcgaccaccaacaccggcgcccaccat
cgcgtcgcagcccctgtccctgcgcccagaggcatgccggccagcagcagggggcgcag
tgcacacgagggggctggacttcgcctgtgatatctacatctgggcgcccttggccggg
acttgtggggtccttctcctgtcactggttatcaccctttactgcaaacggggcagaaa
gaaactcctgtatatattcaaacaaccatttatgagaccagtacaaactactcaagagg
aagatggctgtagctgccgatttccagaagaagaagaaggaggatgtgaactgagagtg
aagttcagcaggagcgcagacgcccccgcgtaccagcagggccagaaccagctctataa
cgagctcaatctaggacgaagagaggagtacgatgttttggacaagagacgtggccggg
accctgagatggggggaaagccgagaaggaagaaccctcaggaaggcctgtacaatgaa
ctgcagaaagataagatggcggaggcctacagtgagattgggatgaaaggcgagcgccg
gaggggcaaggggcacgatggcctttaccagggtctcagtacagccaccaaggacacct
acgacgcccttcacatgcaggccctgccccctcgcgctgtttctttgagcaaaatgcta
aagaaaagaagccctcttacaacaggggtctatgtgaaaatgcccccaacagagccaga
atgtgaaaagcaatttcagccttattttattcccatcaatggcgtctacgttaagatgc
cacccacggagccagaatgcgaaaaacagtttcaaccatatttcataccaataaacggg
gtctatgttaaaatgccgcctactgagcctgagtgtgaaaaacaattccagccatattt
tatacccataaattgtacagagggcagaggaagtctgctaacatgcggtgacgtcgagg
agaatcctggccca.

In some embodiments, the fusion peptide is an anti-CD19 CAR-CT including a CT domain having one YVKM motif. In some embodiments, an anti-CD19 CAR-CT including one CT domain is CD19sc-Fv(4BBZ)-CAR-CT, including ectodomain components of CD8, CD137, and CD3, in addition to CT, and having an amino acid sequence:

(SEQ ID NO: 61)
DIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDYWGQGT
SVTVISTTTPAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRAVSLSKM
LKKRSPLTTGVYVKMPPTEPECEKQFQPYFIPIN.

In some embodiments, an anti CD19sc-Fv(4BBZ)-CAR-CT, including ectodomain components of CD8, CD137, and CD3, in addition to CT, has a nucleic acid sequence:

(SEQ ID NO: 62)
gacatccagatgacacagactacatcctccctgtctgcctctctgggagacagagtcac
catcagttgcagggcaagtcaggacattagtaagtatttaaattggtatcagcagaaac
cagatggaactgttaaactcctgatctaccatacatcaagattacactcaggagtccca
tcaaggttcagtggcagtgggtctggaacagattattctctcaccattagcaacctgga
gcaagaagatattgccacttacttttgccaacagggtaatacgcttccgtacacgttcg
gaggggggaccaagctggagatcacaggtggcggtggctcgggcggtggtgggtcgggt
ggcggcggatctgaggtgaaactgcaggagtcaggacctggcctggtggcgccctcaca
gagcctgtccgtcacatgcactgtctcaggggtctcattacccgactatggtgtaagct
ggattcgccagcctccacgaaagggtctggagtggctgggagtaatatggggtagtgaa
accacatactataattcagctctcaaatccagactgaccatcatcaaggacaactccaa
gagccaagttttcttaaaaatgaacagtctgcaaactgatgacacagccatttactact
gtgccaaacattattactacggtggtagctatgctatggactactggggccaaggaacc
tcagtcaccgtcacctcaaccacgacgccagcgccgcgaccaccaacaccggcgcccac
catcgcgtcgcagcccctgtccctgcgcccagaggcatgccggccagcagcagggggcg
cagtgcacacgagggggctggacttcgcctgtgatatctacatctgggcgcccttggcc
gggacttgtggggtccttctcctgtcactggttatcaccctttactgcaaacggggcag
aaagaaactcctgtatatattcaaacaaccatttatgagaccagtacaaactactcaag
aggaagatggctgtagctgccgatttccagaagaagaagaaggaggatgtgaactgaga
gtgaagttcagcaggagcgcagacgcccccgcgtaccagcagggccagaaccagctcta
taacgagctcaatctaggacgaagagaggagtacgatgttttggacaagcgacgtggcc
gggaccctgagatggggggaaagccgagaaggaagaaccctcaggaaggcctgtacaat
gaactgcagaaagataagatggcggaggcctacagtgagattgggatgaaaggcgagcg
ccggaggggcaaggggcacgatggcctttaccagggtctcagtacagccaccaaggaca
cctacgacgcccttcacatgcaggccctgccccctcgcgctgtttctttgagcaaaatg
ctaaagaaaagaagccctcttacaacaggggtctatgtgaaaatgcccccaacagagcc
agaatgtgaaaagcaatttcagccttattttattcccatcaat.

In some embodiments, an anti-CD19 CAR-CT including a CT domain having two YVKM motifs is CD19sc-Fv(4BBZ)-CAR-CT(2), including ectodomain components of CD8, CD137, and CD3, in addition to CT(2), and having an amino acid sequence:

(SEQ ID NO: 63)
DIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDYWGQGT
SVTVTSTTTPAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRAVSLSKM
LKKRSPLTTGVYVKMPPTEPECEKQFQPYFIPINGVYVKMPPTEPECEKQFQPYFIPIN.

The YVKM motifs are underlined.

In some embodiments, an anti CD19sc-Fv(4BBZ)-CAR-CT, including ectodomain components of CD8, CD137, and CD3, in addition to CT, has a nucleic acid sequence:

(SEQ ID NO: 64)
gacatccagatgacacagactacatcctccctgtctgcctctctgggagacagagtcac
catcagttgcagggcaagtcaggacattagtaagtatttaaattggtatcagcagaaac
cagatggaactgttaaactcctgatctaccatacatcaagattacactcaggagtccca
tcaaggttcagtggcagtgggtctggaacagattattctctcaccattagcaacctgga
gcaagaagatattgccacttacttttgccaacagggtaatacgcttccgtacacgttcg
gaggggggaccaagctggagatcacaggtggcggtggctcgggcggtggtgggtcgggt
ggcggcggatctgaggtgaaactgcaggagtcaggacctggcctggtggcgccctcaca
gagcctgtccgtcacatgcactgtctcaggggtctcattacccgactatggtgtaagct
ggattcgccagcctccacgaaagggtctggagtggctgggagtaatatggggtagtgaa
accacatactataattcagctctcaaatccagactgaccatcatcaaggacaactccaa
gagccaagttttcttaaaaatgaacagtctgcaaactgatgacacagccatttactact
gtgccaaacattattactacggtggtagctatgctatggactactggggccaaggaacc
tcagtcaccgtcacctcaaccacgacgccagcgccgcgaccaccaacaceggcgcccac
catcgcgtcgcagcccctgtccctgcgcccagaggcatgccggccagcagcagggggcg
cagtgcacacgagggggctggacttcgcctgtgatatctacatctgggcgcccttggcc
gggacttgtggggtccttctcctgtcactggttatcaccctttactgcaaacggggcag
aaagaaactcctgtatatattcaaacaaccatttatgagaccagtacaaactactcaag
aggaagatggctgtagctgccgatttccagaagaagaagaaggaggatgtgaactgaga
gtgaagttcagcaggagcgcagacgcccccgcgtaccagcagggccagaaccagctcta
taacgagctcaatctaggacgaagagaggagtacgatgttttggacaagcgacgtggcc
gggaccctgagatggggggaaagccgagaaggaagaaccctcaggaaggcctgtacaat
gaactgcagaaagataagatggcggaggcctacagtgagattgggatgaaaggcgagcg
ccggaggggcaaggggcacgatggcctttaccagggtctcagtacagccaccaaggaca
cctacgacgcccttcacatgcaggccctgccccctcgcgctgtttctttgagcaaaatg
ctaaagaaaagaagccctcttacaacaggggtctatgtgaaaatgcccccaacagagcc
agaatgtgaaaagcaatttcagccttattttattcccatcaatggcgtctacgttaaga
tgccacccacggagccagaatgcgaaaaacagtttcaaccatatttcataccaataaac.

In some embodiments, an anti-CD19 CAR-CT including a CT domain having two YVKM motifs is CD19sc-Fv(4BBZ)-CAR-CT(2), including ectodomain components of CD8, CD137, and CD3, in addition to CT(2), and having an amino acid sequence:

(SEQ ID NO: 65)
DIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDYWGQGT
SVTVTSTTTPAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRAVSLSKM
LKKRSPLTTGVYVKMPPTEPECEKQFQPYFIPINGVYVKMPPTEPECEKQFQPYFIPIN
GVYVKMPPTEPECEKQFQPYFIPIN.

In some embodiments, an anti CD19sc-Fv(4BBZ)-CAR-CT, including ectodomain components of CD8, CD137, and CD3, in addition to CT, has a nucleic acid sequence:

(SEQ ID NO: 66)
gacatccagatgacacagactacatcctccctgtctgcctctctgggagacagagtcac
catcagttgcagggcaagtcaggacattagtaagtatttaaattggtatcagcagaaac
cagatggaactgttaaactcctgatctaccatacatcaagattacactcaggagtccca
tcaaggttcagtggcagtgggtctggaacagattattctctcaccattagcaacctgga
gcaagaagatattgccacttacttttgccaacagggtaatacgcttccgtacacgttcg
gaggggggaccaagctggagatcacaggtggcggtggctcgggcggtggtgggtcgggt
ggcggcggatctgaggtgaaactgcaggagtcaggacctggcctggtggcgccctcaca
gagcctgtccgtcacatgcactgtctcaggggtctcattacccgactatggtgtaagct
ggattcgccagcctccacgaaagggtctggagtggctgggagtaatatggggtagtgaa
accacatactataattcagctctcaaatccagactgaccatcatcaaggacaactccaa
gagccaagttttcttaaaaatgaacagtctgcaaactgatgacacagccatttactact
gtgccaaacattattactacggtggtagctatgctatggactactggggccaaggaacc
tcagtcaccgtcacctcaaccacgacgccagcgccgcgaccaccaacaccggcgcccac
catcgcgtcgcagcccctgtccctgcgcccagaggcatgccggccagcagcagggggcg
cagtgcacacgagggggctggacttcgcctgtgatatctacatctgggcgcccttggcc
gggacttgtggggtccttctcctgtcactggttatcaccctttactgcaaacggggcag
aaagaaactcctgtatatattcaaacaaccatttatgagaccagtacaaactactcaag
aggaagatggctgtagctgccgatttccagaagaagaagaaggaggatgtgaactgaga
gtgaagttcagcaggagcgcagacgcccccgcgtaccagcagggccagaaccagctcta
taacgagctcaatctaggacgaagagaggagtacgatgttttggacaagcgacgtggcc
gggaccctgagatggggggaaagccgagaaggaagaaccctcaggaaggcctgtacaat
gaactgcagaaagataagatggcggaggcctacagtgagattgggatgaaaggcgagcg
ccggaggggcaaggggcacgatggcctttaccagggtctcagtacagccaccaaggaca
cctacgacgcccttcacatgcaggccctgccccctcgcgctgtttctttgagcaaaatg
ctaaagaaaagaagccctcttacaacaggggtctatgtgaaaatgcccccaacagagcc
agaatgtgaaaagcaatttcagccttattttattcccatcaatggcgtctacgttaaga
tgccacccacggagccagaatgcgaaaaacagtttcaaccatatttcataccaataaac
ggggtctatgttaaaatgccgcctactgagcctgagtgtgaaaaacaattccagccata
ttttatacccataaat.

b. Programed Death Protein 1 (PD1) Domains

In some embodiments, fusion peptides include a CT domain having one or more YVKM motifs and polypeptide sequences encoding a programmed death protein 1 (PD1) domain. Therefore, in some embodiments, CT fusion peptides include one or more PD1 ectodomain peptide sequences.

Programmed cell death protein 1 (PD1) is an inhibitory receptor that is expressed by all T cells during activation. It regulates T cell effector functions during various physiological responses, including acute and chronic infection, cancer and autoimmunity, and in immune homeostasis. PD1 often shows high and sustained expression levels during persistent antigen encounter, which can occur in the setting of chronic infections and cancer. In these settings, PD1 can limit protective immunity. In addition to being expressed by conventional T cells, PD1 is expressed by regulatory T cells, B cells, natural killer cells and some myeloid cell populations. However, compared with conventional T cells, less is known about how PD1 inhibitory signals regulate these cell types. Programmed cell death 1 ligand 1 (PDL1) shows broad expression on both hematopoietic and non-hematopoietic cells, positioning the PD1 pathway as a key regulator of immune cell functions in both secondary lymphoid organs and in non-lymphoid tissues. PD1 limits the activation and function of potentially pathogenic self-reactive CD4+ and CD8+ T cells, and PDL1 can shield target organs from autoimmune attack. Some anticancer drugs, called immune checkpoint inhibitors, are used to block PD-1. When this protein is blocked, the “brakes” on the immune system are released and the ability of T cells to kill cancer cells is increased.

In some embodiments, CT fusion peptides include a PD1 molecule having a cytoplasmic domain and/or transmembrane domain replaced or altered to include a CT domain having one or more YVKM motifs, for example, having an amino acid sequence of or including any one or more of SEQ ID NOs: 3, 5, 7, or 9-50, or 108-123 or functional fragment or variant thereof.

An exemplary PD1-CT fusion includes the ectodomain (and optionally transmembrane domain) of a human PD1 protein and cytoplasmic regions (and optionally the transmembrane region) of CTLA-4 including a CT domain. For example, in some embodiments, a PD1-CT fusion peptide has an amino acid sequence of:

(SEQ ID NO: 74)
FLDSPDRPWNPPTFSPALLVVTEGDNATFTCSFSNTSESFVLNWYRMSPSNQTDKLAAF
PEDRSQPGQDCRFRVTQLPNGRDFHMSVVRARRNDSGTYLCGAISLAPKAQIKESLRAE
LRVTERRAEVPTAHPSPSPRPAGQFQTLVVGVVGGLLGSLVLLVWVLAVIAVSLSKMLK
KRSPLTTGVYVKMPPTEPECEKQFQPYFIPIN.

An exemplary nucleic acid sequence for a PD1-CT fusion protein is:

(SEQ ID NO: 75)
ttcttagactccccagacaggccctggaacccccccaccttctccccagccctgctcgt
ggtgaccgaaggggacaacgccaccttcacctgcagcttctccaacacatcggagagct
tcgtgctcaactggtaccgcatgagccccagcaaccagacggacaagctggccgccttc
cccgaggaccgcagccagcccggccaggactgccgcttccgtgtcacacaactgcccaa
cgggcgtgacttccacatgagcgtggtcagggcccggcgcaatgacagcggcacctacc
tctgtggggccatctccctggcccccaaggcgcagatcaaagagagcctgcgggcagag
ctcagggtgacagagagaagggcagaagtgcccacagcccaccccagcccctcacctag
gccagccggccagttccaaaccctggtggttggtgtcgtgggcggcctgctgggcagcc
tggtgctgctagtctgggtcctggccgtcatcgctgtttctttgagcaaaatgctaaag
aaaagaagccctcttacaacaggggtctatgtgaaaatgcccccaacagagccagaatg
tgaaaagcaatttcagccttattttattcccatcaat.

An exemplary PD1-CT(2) fusion peptide includes the ectodomain (and optionally transmembrane domain) of a human PD1 protein and optionally two copies of a peptide including the YVKM motif (and optionally a transmembrane region) of a CT peptide from CTLA-4 tail. For example, in some embodiments, a PD1-CT(2) fusion peptide has a CT domain having two YVKM motifs an amino acid sequence of:

(SEQ ID NO: 76)
FLDSPDRPWNPPTFSPALLVVTEGDNATFTCSFSNTSESFVLNWYRMSPSNQTDKLAAF
PEDRSQPGQDCRFRVTQLPNGRDFHMSVVRARRNDSGTYLCGAISLAPKAQIKESLRAE
LRVTERRAEVPTAHPSPSPRPAGQFQTLVVGVVGGLLGSLVLLVWVLAVIAVSLSKMLK
KRSPLTTGVYVKMPPTEPECEKQFQPYFIPINGVYVKMPPTEPECEKQFQPYFIPIN.

An exemplary nucleic acid sequence for a PD1-CT(2) fusion protein is:

(SEQ ID NO: 77)
Ttcttagactccccagacaggccctggaacccccccaccttctccccagccctgctcgt
ggtgaccgaaggggacaacgccaccttcacctgcagcttctccaacacatcggagagct
tcgtgctcaactggtaccgcatgagccccagcaaccagacggacaagctggccgccttc
cccgaggaccgcagccagcccggccaggactgccgcttccgtgtcacacaactgcccaa
cgggcgtgacttccacatgagcgtggtcagggcccggcgcaatgacagcggcacctacc
tctgtggggccatctccctggcccccaaggcgcagatcaaagagagcctgcgggcagag
ctcagggtgacagagagaagggcagaagtgcccacagcccaccccagcccctcacctag
gccagccggccagttccaaaccctggtggttggtgtcgtgggcggcctgctgggcagcc
tggtgctgctagtctgggtcctggccgtcatcgctgtttctttgagcaaaatgctaaag
aaaagaagccctcttacaacaggggtctatgtgaaaatgcccccaacagagccagaatg
tgaaaagcaatttcagccttattttattcccatcaatggcgtctacgttaagatgccac
ccacggagccagaatgcgaaaaacagtttcaaccatatttcataccaataaac.

An exemplary PD1-CT(3) fusion peptide includes the ectodomain (and optionally transmembrane domain) of a human PD1 protein and optionally three copies of a peptide including the YVKM motif (and optionally a transmembrane region) of a CT domain from CTLA-4. For example, in some embodiments, a PD1-CT(3) fusion peptide has a CT domain having three YVKM motifs and an amino acid sequence of:

(SEQ ID NO: 126)
FLDSPDRPWNPPTFSPALLVVTEGDNATFTCSFSNTSESFVLNWYRMSPSNQTDKLAAF
PEDRSQPGQDCRFRVTQLPNGRDFHMSVVRARRNDSGTYLCGAISLAPKAQIKESLRAE
LRVTERRAEVPTAHPSPSPRPAGQFQTLVVGVVGGLLGSLVLLVWVLAVIAVSLSKMLK
KRSPLTTGVYVKMPPTEPECEKQFQPYFIPINGVYVKMPPTEPECEKQFQPYFIPINGV
YVKMPPTEPECEKQFQPYFIPIN.

An exemplary nucleic acid sequence for a PD1-CT(3) fusion protein is:

(SEQ ID NO: 78)
Ttcttagactccccagacaggccctggaacccccccaccttctccccagccctgctcgt
ggtgaccgaaggggacaacgccaccttcacctgcagcttctccaacacatcggagagct
tcgtgctcaactggtaccgcatgagccccagcaaccagacggacaagctggccgccttc
cccgaggaccgcagccagcccggccaggactgccgcttccgtgtcacacaactgcccaa
cgggcgtgacttccacatgagcgtggtcagggcccggcgcaatgacagcggcacctacc
tctgtggggccatctccctggcccccaaggcgcagatcaaagagagcctgcgggcagag
ctcagggtgacagagagaagggcagaagtgcccacagcccaccccagcccctcacctag
gccagccggccagttccaaaccctggtggttggtgtcgtgggcggcctgctgggcagcc
tggtgctgctagtctgggtcctggccgtcatcgctgtttctttgagcaaaatgctaaag
aaaagaagccctcttacaacaggggtctatgtgaaaatgcccccaacagagccagaatg
tgaaaagcaatttcagccttattttattcccatcaatggcgtctacgttaagatgccac
ccacggagccagaatgcgaaaaacagtttcaaccatatttcataccaataaacggggtc
tatgttaaaatgccgcctactgagcctgagtgtgaaaaacaattccagccatattttat
acccataaat.

### c. 2A Peptides

In some embodiments, fusion peptides include a CT domain having one or more YVKM motifs and polypeptide sequences encoding a viral 2A region. Therefore, in some embodiments, CT fusion peptides include one or more 2A peptide sequences, typically at the Carboxyl (C) terminus of the fusion peptide.

T2A peptides are 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was recently discovered to be ribosome skipping the formation of a glycyl-prolyl peptide bond at the C-terminus of the 2A20). A highly conserved sequence GDVEXNPGP (SEQ ID NO:87) is shared by different 2As at the C-terminus, and is essential for the creation of steric hindrance and ribosome skipping. There are three possibilities for a 2A-mediated skipping event:
1—Successful skipping and recommencement of translation results in two “cleaved” proteins: the protein upstream of the 2A is attached to the complete 2A peptide except for the C-terminal proline, and the protein downstream of the 2A is attached to one proline at the N-terminus;
2—Successful skipping but ribosome fall-off and discontinued translation results in only the protein upstream of 2A; and
3—Unsuccessful skipping and continued translation resulting in a fusion protein.

Overall, 2A peptides lead to relatively high levels of downstream protein expression compared to other strategies for multi-gene co-expression, and they are small in size thus bearing a lower risk of interfering with the function of co-expressed genes. 2A peptides have also been successfully employed by several different groups for polycistronic and bi-cistronic multigene expression.

Therefore, in some embodiments, CT domains for including within fusion proteins are coupled to one or more 2A polypeptide sequences.

An exemplary amino acid sequence for a T2A sequence is:

(SEQ ID NO: 67)
GSGSGEGRGSLLTCGDVEENPGP.

An exemplary amino acid sequence for a CT domain including a T2A sequence is:

(SEQ ID NO: 68)
AVSLSKMLKKRSPLTTGVYVKMPPTEPECEKQFQPYFIPINGSGSGEGR
GSLLTCGDVEENPGP.

An exemplary nucleic acid sequence for a CT domain including a T2A sequence is:

(SEQ ID NO: 69)
gctgtttctttgagcaaaatgctaaagaaaagaagccctcttacaacaggggtctatgt
gaaaatgcccccaacagagccagaatgtgaaaagcaatttcagccttattttattccca
tcaatggatccggcagtggagagggcagaggaagtctgctaacatgcggtgacgtcgag
gagaatcctggccca.

An exemplary amino acid sequence for a CT(2) domain (including two YVKM motifs) and a T2A sequence is:

(SEQ ID NO: 70)
AVSLSKMLKKRSPLTTGVYVKMPPTEPECEKQFQPYFIPINGVYVKMPP
TEPECEKQFQPYFIPINGSGSGEGRGSLLTCGDVEENPGP.
The T2A region is underlined.

An exemplary nucleic acid sequence for a CT(2) domain including a T2A sequence is:

(SEQ ID NO: 71)
Attgatgggaataaaataaggctgaaattgcttttcacattctggctctgttgggggca
ttttcacatagacccctgttgtaagagggcttcttttctttagcattttgctcaaagaa
acagcggcgtctacgttaagatgccacccacggagccagaatgcgaaaaacagtttcaa
ccatatttcataccaataaacggatccggcagtggagagggcagaggaagtctgctaac
atgcggtgacgtcgaggagaatcctggccca.

An exemplary amino acid sequence for a CT(3) domain (including three YVKM motifs) and a T2A sequence is:

(SEQ ID NO: 72)
AVSLSKMLKKRSPLTTGVYVKMPPTEPECEKQFQPYFIPINGVYVKMPP
TEPECEKQFQPYFIPINGVYVKMPPTEPECEKQFQPYFIPINGSGSGEG
RGSLLTCGDVEENPGP.
The T2A region is underlined.

An exemplary nucleic acid sequence for a CT(3) domain including a T2A sequence is:

(SEQ ID NO: 73)
gctgtttctttgagcaaaatgctaaagaaaagaagccctcttacaacaggggtctatgt
gaaaatgcccccaacagagccagaatgtgaaaagcaatttcagccttattttattccca
tcaatggcgtctacgttaagatgccacccacggagccagaatgcgaaaaacagtttcaa
ccatatttcataccaataaacggggtctatgttaaaatgccgcctactgagcctgagtg
tgaaaaacaattccagccatattttatacccataaatggatccggcagtggagagggca
gaggaagtctgctaacatgcggtgacgtcgaggagaatcctggccca.

Exemplary fusion peptides including T2A peptides include CAR-CT-T2A constructs.

d. Other Protein Domains

Any of the disclosed recombinant proteins can include one or more additional domains. For example, any of the disclosed proteins can include one or more linkers or spacers. The term “linker” as used herein includes, without limitation, peptide linkers. The peptide linker can be any size provided it does not interfere with the binding of the epitope by the variable regions. In some embodiments, the linker includes one or more glycine and/or serine amino acid residues. In some embodiments, the linker includes a glycine-glutamic acid di-amino acid sequence. For example, a linker can include 4-8 amino acids. In a particular embodiment, a linker includes the amino acid sequence GQSSRSS (SEQ ID NO:79). In some forms, the linker includes one, two or more copies the amino acid sequence GGGGS (SEQ ID NO:125). In another embodiment, a linker includes 15-20 amino acids, for example 18 amino acids. Other flexible linkers include, but are not limited to, the amino acid sequences Gly-Ser, Gly-Ser-Gly-Ser (SEQ ID NO:80), Ala-Ser, Gly-Gly-Gly-Ser (SEQ ID NO:81), (Gly₄-Ser)₂ (SEQ ID NO:82) and (Gly₄-Ser)₄ (SEQ ID NO:83), (Gly-Gly-Gly-Ser)₂ (SEQ ID NO: 84) and (Gly-Gly-Gly-Gly-Ser)₃ (SEQ ID NO:85).

The linkers can be used to link or connect two domains, regions, or sequences of a fusion protein. Molecular biology techniques have developed so that therapeutic proteins can be genetically engineered to be expressed by microorganisms. The gram negative bacterium, Escherichia coli, is a versatile and valuable organism for the expression of therapeutic proteins. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some embodiments, the compositions disclosed herein include expression or solubility enhancing amino acid sequence. In some embodiments, the expression or solubility enhancing amino acid sequence is cleaved prior administration of the composition to a subject in need thereof. The expression or solubility enhancing amino acid sequence can be cleaved in the recombinant expression system, or after the expressed protein in purified.

### C. Nucleic Acids

Nucleic acids and vectors encoding or expressing CT proteins and CT fusion proteins are also described.

### 1. Isolated Nucleic Acid Molecules of CT or CT-Fusion Peptides

Isolated nucleic acid sequences encoding CT polypeptides and CT fusion peptides are disclosed. In some embodiments, the isolated nucleic acid sequences encode a CAR-CT, including a CAR fused with a CT domain having one, two or three YVKM motifs. In preferred embodiments, an isolated nucleic acid sequence encodes a CAR fused with a CT domain having two YVKM motifs.

The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding a CT polypeptide or CT fusion peptide. Thus, nucleic acids encoding SEQ ID NOS:2 and 4 and 6, and fragments and variants thereof, in sense and antisense, and in single stranded and double stranded forms, are provided. The nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### 2. Vectors Expressing or Encoding CT or CT-Fusion Peptides

In some embodiments, nucleic acids encoding CT or CT-Fusion Peptides are present within vectors. In some embodiments, the vectors encode or express a CAR-CT, including a CAR fused with a CT domain having one, two or three YVKM motifs. In preferred embodiments, a vector encodes or expresses a CAR fused with a CT domain having two YVKM motifs.

Vectors including an isolated polynucleotide encoding a CT polypeptide and/or fusion polypeptide including a CT domain having one or more YVKM motifs and one or more heterologous domains for the expression of a CT or CT fusion peptide within a host cell are described.

The term “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some embodiments, a vector is a DNA plasmid. One of ordinary skill in the art can construct a vector of the application through standard recombinant techniques in view of the present disclosure.

In some embodiments, the vector including nucleic acids encoding a CT domain or CT fusion protein is an expression vector. The term “expression vector” refers to any type of genetic construct including a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some embodiments, vectors contain one or more regulatory sequences. The term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically include an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. complete baculovirus expression system (Thermo Fisher Scientific), which can be used for production and/or expression in insect cells; pcDNA™. or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. In some embodiments, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

In some embodiments the vector is a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated (and replaced by a nucleic acid encoding a PC1-CTT polypeptide or another therapeutic polypeptide as described herein), arenavirus such as lymphocytic choriomeningitidis virus (LCMV), e.g., clone 13 strain or MP strain, and arenavirus such as Junin virus e.g., Candid #1 strain, etc.

In some embodiments, the viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53. In some embodiments, a recombinant viral vector is prepared using methods known in the art in view of the present disclosure. For example, in view of the degeneracy of the genetic code, several nucleic acid sequences can be designed that encode the same polypeptide. In some embodiments, a polynucleotide encoding a CT polypeptide or CT-fusion polypeptide is codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

In some embodiments, the vectors, e.g., a DNA plasmid or a viral vector (particularly an adenoviral vector), include any regulatory elements to establish conventional function(s) of the vector, including but not limited to replication and expression of the CT polypeptide or CT-fusion polypeptide encoded by the polynucleotide sequence of the vector.

### 3. Regulatory Elements

In some embodiments, the disclosed nucleic acids, including RNAs and DNAs such as DNA vectors expressing or encoding a CT polypeptide or CT-fusion polypeptide include one or more regulatory elements.

Regulatory elements include, but are not limited to, a promoter, an enhancer, a polyadenylation signal, translation stop codon, a ribosome binding element, a transcription terminator, selection markers, origin of replication, etc. An isolated nucleic acid can be, and a vector can include, one or more expression cassettes. An “expression cassette” is part of a nucleic acid such as a vector that directs the cellular machinery to make RNA and protein. An expression cassette typically includes three components: a promoter sequence, an open reading frame, and a 3-untranslated region (UTR) optionally including a polyadenylation signal. An open reading frame (ORF) is a reading frame that contains a coding sequence of a protein of interest (e.g., CT polypeptide, or CT-fusion polypeptide, etc.) from a start codon to a stop codon. Regulatory elements of the expression cassette can be operably linked to a polynucleotide sequence encoding a PC1-CTT polypeptide or other therapeutic polypeptide.

As used herein, the term “operably linked” is to be taken in its broadest reasonable context, and refers to a linkage of polynucleotide (or polypeptide, etc.) elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

a. Promotors

The disclosed nucleic acids, including vectors, can include a promoter sequence, preferably within an expression cassette, to control expression of a CT polypeptide or CT-fusion polypeptide. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding a CT polypeptide or CT-fusion polypeptide within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein.

A promoter can also be a tissue specific promoter, such as a kidney specific promoter, preferably a kidney epithelial cell promoter, which can be natural or synthetic. Examples include, but are not limited to, the CDH 16 promoter, which is mostly kidney specific (it is also expressed in the thyroid) (Igarashi, et al., Am J Physiol., 277(4):F599-610 (1999). doi: 10.1152/ajprenal.1999.277.4.F599. PMID: 10516285.); the Pax-8 promoter, which is also expressed primarily in the kidney as well as in the thyroid (Dehbi, et al., EMBO J., 15(16):4297-306 (1996) PMID: 8861958); the aquaporin 2 promoter, which drives expression specifically in principal cells of the renal collecting duct (which are the target of Tolvaptan) (Stricklett, et al., Exp Nephrol., 7(1):67-74 (1999). doi: 10.1159/000020587. PMID: 9892817.), and kidney tubule-specific promoters in association with gene delivery viral vectors (Watanabe, et al., PloS one, vol. 12,3 e0168638 (2017), doi:10.1371/journal.pone.0168638).

In some embodiments, the promoter is a strong eukaryotic promoter, such as cytomegalovirus immediate early (CMV-IE) promoter.

b. Other Expression Control Elements

In some embodiments, the nucleic acids, including vectors, include additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a CT polypeptide or CT-fusion polypeptide within an expression cassette of the vector. Enhancer sequences are regulatory DNA sequences that, when bound by transcription factors, enhance the transcription of an associated gene. An enhancer sequence is preferably located upstream of the polynucleotide sequence encoding a CT polypeptide or CT-fusion polypeptide, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human beta-globin polyadenylation signal. Preferably, a polyadenylation signal is a bovine growth hormone (bGH) polyadenylation signal or a SV40 polyadenylation signal.

Any enhancer sequence known to those skilled in the art in view of the present disclosure can be used. For example, an enhancer sequence can be a human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit beta-globin intron, or any combination thereof. Preferably, an enhancer sequence is a composite sequence of three consecutive elements of the untranslated R-U5 domain of HTLV-1 LTR, rabbit beta-globin intron, and a splicing enhancer, which is referred to herein as “a triple enhancer sequence.”

A vector can include a polynucleotide sequence encoding a signal peptide sequence. Preferably, the polynucleotide sequence encoding the signal peptide sequence is located upstream of the polynucleotide sequence encoding a CT polypeptide or CT-fusion polypeptide. Signal peptides typically direct localization of a protein, facilitate secretion of the protein from the cell in which it is produced, and/or improve expression the therapeutic polypeptide when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding a CT polypeptide or CT-fusion polypeptide, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., a CT polypeptide or CT-fusion polypeptide. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

An expression vector can include a tag sequence, such as those discussed above.

### D. Host Cells

In some embodiments, polypeptides, nucleic acids, or vectors encoding CT polypeptides or CT-fusion polypeptides are present within a host cells. In some embodiments, the cells include nucleic acids or vectors or genes that encode or express a CAR-CT, including a CAR fused with a CT domain having one, two or three YVKM motifs. In preferred embodiments, the cells include nucleic acids or vectors or genes that encode or express a CAR fused with a CT domain having two YVKM motifs. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the CT polypeptides or CT-fusion polypeptides described herein.

In some forms, the cell is from an established cell line, or a primary cell. The term “primary cell,” refers to cells and cell cultures derived from a subject and allowed to grow in vitro for a limited number of passages, i.e. splitting, of the culture.

1. Human cells

In some embodiments, cells are obtained from a human subject. Therefore, human cells expressing and/or including CT-fusion polypeptides are described. In preferred embodiments, the human cells include or express a CAR-CT, including a CAR fused with a CT domain having one, two or three YVKM motifs. In preferred embodiments, the human cells include or express a CAR fused with a CT domain having two YVKM motifs. For example, in some forms, the cells are autologous cells, i.e., cells obtained from a subject prior to introduction of the CT polypeptides or CT-fusion polypeptides, and/or nucleic acids, or vectors encoding CT polypeptides or CT-fusion polypeptides, and re-introduction to the same subject following modification. In other forms, the cells are heterologous cells, i.e., cells obtained from a different subject than the intended recipient. In some forms, the cells are frozen prior to or after introduction of the CT polypeptides or CT-fusion polypeptides, and/or nucleic acids, or vectors encoding CT polypeptides or CT-fusion polypeptides. Methods and compositions for freezing and thawing viable eukaryotic cells are known in the art. In some forms, the cells are autologous immune cells, such as T cells or progenitor cells/stem cells.

In some forms, cells are obtained from a healthy subject. In other forms, cells are obtained from a subject identified as having or at risk of having a disease or disorder, such as cancer and/or an auto-immune disease.

In preferred embodiments, the introduction of the CT polypeptides or CT-fusion polypeptides to the cells occurs through genetic modification of the cells. In some embodiments, genetic modification of the cell includes introduction of nucleic acids, or vectors encoding CT polypeptides or CT-fusion polypeptides to the cell for expression of the CT polypeptides or CT-fusion polypeptides within the cell. In some embodiments, genetic modification of the cell includes transduction with a transposon encoding a CT polypeptide or CT-fusion polypeptide. In an exemplary embodiment, a CAR-CT fusion peptide is introduced into a cell in vitro by transduction of the cell with a nucleic acid encoding a transposon including the CAR-CT. Therefore, genetically modified (transgenic) cells including CT proteins, or CT-fusion proteins according to the described compositions are described.

a. T Cells

In some forms, the cells are human immune cells, such as T cells. Therefore, human T cells that include or express CT-fusion polypeptides are described. In some forms, prior to expansion and genetic modification, T cells are obtained from a diseased or healthy subject. T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some forms, T cells are obtained from a unit of blood collected from a subject using any number of techniques known to the skilled artisan, such as FICOLL™ separation. In one preferred form, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some forms, the cells are washed with phosphate buffered saline (PBS). In some forms, the wash solution lacks calcium and can lack magnesium or can lack many if not all divalent cations. After washing, the cells can be resuspended in a variety of biocompatible buffers, such as, for example, Ca2+-free, Mg2+-free PBS, PLASMALYTE A, or other saline solution with or without buffer. Alternatively, the undesirable components of the apheresis sample are removed and the cells directly resuspended in culture media.

In some forms, T cells are isolated from peripheral blood lymphocytes by lysing the red blood cells and depleting the monocytes, for example, by centrifugation through a PERCOLL™ gradient or by counterflow centrifugal elutriation. In specific forms, a specific subpopulation of T cells, such as CD3+, CD28+, CD4+, CD8+, CD45RA+, and CD45RO+ T cells, is further isolated by positive or negative selection techniques. For example, in some forms, T cells are isolated by incubation with anti-CD3/anti-CD28 (i.e., 3×28)-conjugated beads, such as DYNABEADS® M-450 CD3/CD28 T, for a time period sufficient for positive selection of the desired T cells.

b. Delivery Vehicles

Any of the disclosed compositions including, but not limited to CT polypeptides or CT-fusion proteins, such as CAR-CT and/or nucleic acids, can be delivered to target cells using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for intertissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some embodiments the nanoparticles can have a diameter less than 400 nm, less than 300 nm, or less than 200 nm. The range can be between 50 nm and 300 nm.

Thus, in some embodiments, the delivery vehicles are nanoscale compositions, for example, 10 nm up to, but not including, about 1 micron. However, it will be appreciated that in some embodiments, and for some uses, the particles can be smaller, or larger (e.g., microparticles, etc.). Although many of the compositions disclosed herein are referred to as nanoparticle or nanocarrier compositions, it will be appreciated that in some embodiments and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### 2. Pharmaceutical Compositions

Pharmaceutical compositions containing a genetically modified cell, or a population of genetically modified cells expressing CT polypeptides or CT-fusion proteins, such as CAR-CT, or the polypeptide themselves, or nucleic acid encoding the same are provided.

In some embodiments, the pharmaceutical compositions include one or more of a pharmaceutically acceptable buffer, carrier, diluent or excipients. In some forms, the pharmaceutical compositions include a specific number or population of cells, for example, expanded by culturing and expanding an isolated genetically modified cell (e.g., CAR-CT T cell), e.g., a homogenous population. Therefore, in some embodiments, pharmaceutical compositions include a homogenous population of modified cells including and/or expressing a CT peptide or CT-fusion peptide, such as a CAR-CT. In other forms, the pharmaceutical compositions include populations of cells that contain variable or different genetically modified cells, e.g., a heterogeneous population. In some forms, the pharmaceutical compositions include cells that are bispecific or multi-specific. In some embodiments, the cells have been isolated from a diseased or healthy subject prior to genetic modification to express a CT peptide or CT-fusion peptide, such as a CAR-CT.

The term “pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some embodiments, pharmaceutical compositions include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The pharmaceutical compositions can be formulated for delivery via any route of administration. The term “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods

Methods of using the disclosed compositions including, but not limited to, CT proteins and CT-fusion proteins are provided.

In some embodiments, the methods enhance the efficacy of cell receptor-mediated functions are provided. In particular embodiments, the methods provide enhanced anti-tumor activity through administration of CAR-T cells including CAR-CT fusion peptides. It has been established that, because the endocytosis of CT and the phenomenon of cellular trogocytosis are both highly conserved, they can be utilized to develop a universal approach to reprogram T cell function. The features of the CT were developed into a simple yet versatile approach to enhance Chimeric Antigen Receptor (CAR)-T cell function. For example, in the experiments below, synthetic CTs fused to the C-terminus of the CARs were engineered to control the cellular trogocytosis of the CAR receptor (CAR-CTs). As set forth within experimental data in the Examples, CAR-CT cells, with different numbers of CTs, exhibit progressively reduced trogocytosis and increased cytolysis against cognate cancer cells. Further characterization of CAR-CT cells revealed lower level of trogocytosis and tumor antigen loss, followed by higher resistance to fratricide. Compared with CAR-T cells without CT, CAR-CT cells display significantly lower tonic signaling features and increased responsiveness to antigen stimulation revealed by mRNA-sequencing.

As set forth in the Examples below, in the disclosed experiments, CAR-2CT cells have the most durable in vivo anti-tumor effect, substantially more potent than CAR-1CT, CAR-3CT or control CAR-T cells. In vivo immune characterization showed that CAR-2CT cells have the strongest central memory phenotypes among the four groups and are also persistent. Together, these data here provide a distinct approach to engineer CAR-T cells with functional reprogramming via synthetic protein tails of CTLA-4. Furthermore, the enhanced CAR-T function is (i) independent of CAR types, (ii) can be applied to a broad range of cell therapy, and (iii) can be used in combination with other engineering approaches.

### A. Methods of Treatment

Methods of treatment including cells and other therapeutic agents including CT polypeptides and CT-fusion polypeptides are described. In preferred embodiments, the methods include Adoptive Cell Therapy (ACT) employing T cells expressing recombinant CAR-CT fusion proteins. The CAR T cells including CAR-CT fusion proteins have enhanced anti-tumor activity. For example, the CAR T cells including CAR-CT fusion proteins show reduced trogocytosis and fratricide, enhanced retention of tumor antigen, and enhanced memory phenotype progression as compared to CAR T cells including CAR proteins in the absence of CT.

An exemplary method involves treating a subject (e.g., a human) having a disease, disorder, or condition by administering to the subject an effective amount of a pharmaceutical composition including genetically-modified cells including CT polypeptides and/or CT-fusion polypeptides. In some embodiments, the methods administer genetically manipulated T cells engineered to express recombinant CAR-CT fusion proteins to a subject (e.g., a human) having a disease, disorder, or condition in an amount effective to treat the disease, disorder, or condition. For example, in some embodiments, the methods treat a disease or disorder associated with an elevated expression or specific expression of an antigen by administering to the subject an effective amount of a pharmaceutical composition including cells modified to express recombinant CAR-CT fusion proteins. In some embodiments, the methods treat a subject having a disease, disorder, or condition associated with an elevated expression or specific expression of an antigen by administering to the subject an effective amount of a pharmaceutical composition including T cells modified to contain a CAR-CT that targets the antigen.

Methods of using CT-fusion proteins, such as a CAR-CT, to treat a disease or disorder by are provided. Typically the methods enhance ACT, for example, by providing CAR-CT-bearing T cells with enhanced therapeutic efficacy in vivo. The CAR-CT-T cells have prolonged survival/serum residency time in vivo relative to CAR-T cells lacking the CT fusion domain. Methods of treating a subject having a disease, disorder, or condition including administering to the subject an effective amount of a pharmaceutical composition including live, viable cells engineered to express a CAR-CT and/or another CT-fusion protein are provided. In some embodiments, when the methods treat a subject having a disease, disorder, or condition associated with an elevated expression or specific expression of an antigen, the methods include administering to the subject an effective amount of a T cell modified to express a CAR-CT that targets the antigen. For example, in some forms, the methods treat a subject having a disease, disorder, or condition by administering to the subject an effective amount of a pharmaceutical composition having a genetically modified cell, where the cell is modified by introducing to the cell:
(i) a vector, optionally including a transposon encoding a CAR-CT and/or other CT-fusion protein; and
(ii) causing the CAR-CT and/or other CT-fusion protein to be expressed by the cell. The cell can have been isolated from the subject having the disease, disorder, or condition, or from a healthy donor, prior to genetic modification.

### 1. Diseases to be Treated

Methods of treating diseases and/or disorders in a subject in need thereof are provided. The subject to be treated can have a disease, disorder, or condition such as but not limited to, cancer, an immune system disorder such autoimmune disease, an inflammatory disease, a neuronal disorder, HIV/AIDS, diabetes, a cardiovascular disease, an infectious disease, or combinations thereof. The disease, disorder, or condition can be associated with an elevated expression or specific expression of an antigen.

a. Cancer

In some embodiments, the methods treat or prevent cancer. In some forms, the methods treat or prevent cancer or other proliferative disease or disorder in a subject identified as having, or at risk of having cancer or other proliferative disease or disorder. Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell.,144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

TABLE 3
Exemplary cancers for which the CAR-CT of the disclosed methods
and compositions can target a specific or an associated antigen.

| | | | | |
|---|---|---|---|---|
| Acute | Acute | Adrenocortical | AIDS-Related | Kaposi |
| Lymphoblastic | Myeloid | Carcinoma | Cancers | Sarcoma |
| Leukemia (ALL) | Leukemia (AML) | | | |
| AIDS-Related | Primary CNS | Anal Cancer | Appendix | Astrocytomas |
| Lymphoma | Lymphoma | | Cancer | |
| | | | (Gastrointestinal | |
| | | | Carcinoid Tumors) | |
| Atypical | Brain | Basal Cell | Bile Duct | Bladder |
| Teratoid/ | Cancer | Carcinoma | Cancer | Cancer |
| Rhabdoid | | of the Skin | | |
| Tumor | | | | |
| Bone Cancer | Brain | Breast | Bronchial | Burkitt |
| (includes Ewing | Tumors | Cancer | Tumors | Lymphoma |
| Sarcoma and | | | | |
| Osteosarcoma | | | | |
| and Malignant | | | | |
| Fibrous | | | | |
| Histiocytoma) | | | | |
| Non-Hodgkin | Carcinoid | Carcinoma of | Cardiac (Heart) | Embryonal |
| Lymphoma | Tumors | Unknown Primary | Tumors | Tumors |
| Germ Cell | Primary CNS | Cervical | Cholangio- | Chordoma |
| Tumor | Lymphoma | Cancer | carcinoma | |
| Chronic | Chronic | Chronic | Colorectal | Cranio- |
| Lymphocytic | Myelogenous | Myeloproliferative | Cancer | pharyngioma |
| Leukemia (CLL) | Leukemia (CML) | Neoplasms | | |
| Cutaneous | Ductal | Endometrial | Ependymoma | Esophageal |
| T-Cell | Carcinoma In | Cancer | | Cancer |
| Lymphoma | Situ (DCIS) | | | |
| (Mycosis | | | | |
| Fungoides | | | | |
| and Sézary | | | | |
| Syndrome) | | | | |
| Esthesioneuro- | Ewing | Extracranial | Eye | Intraocular |
| blastoma | Sarcoma | Germ Cell Tumor | Cancer | Melanoma |
| Fallopian | Fibrous | Osteosarcoma | Gallbladder | Gastric |
| Tube Cancer | Histiocytoma | | Cancer | Cancer |
| | of Bone | | | |
| Stomach | Gastrointestinal | Gastrointestinal | Central Nervous | Extracranial |
| Cancer | Carcinoid | Stromal Tumors | System Germ | Germ Cell |
| | Tumor | (GIST) | Cell Tumors | Tumors |
| Extragonadal | Ovarian Germ | Testicular | Gestational | Hairy Cell |
| Germ Cell | Cell Tumors | Cancer | Trophoblastic | Leukemia |
| Tumors | | | Disease | |
| Head and | Heart | Hepatocellular | Histiocytosis | Hodgkin |
| Neck Cancer | Tumors | (Liver) Cancer | (Langerhans | Lymphoma |
| | | | Cell) | |
| Hypopharyngeal | Intraocular | Islet Cell | Pancreatic | Kidney |
| Cancer | Melanoma | Tumors | Neuroendocrine | Cancer |
| | | | Tumors | |
| Renal Cell | Langerhans | Laryngeal | Leukemia | Lip and Oral |
| Cancer | Cell | Cancer | | Cavity Cancer |
| | Histiocytosis | | | |
| Liver | Lung Cancer | Lymphoma | Male Breast | Malignant |
| Cancer | (Non-Small | | Cancer | Fibrous |
| | Cell and Small | | | Histiocytoma |
| | Cell) | | | of Bone and |
| | | | | Osteosarcoma |
| Melanoma | Intraocular | Merkel Cell | Malignant | Metastatic |
| | (Eye) Melanoma | Carcinoma | Mesothelioma | Cancer |
| | | (Skin Cancer) | | |
| Metastatic | Midline Tract | Mouth | Multiple | Multiple |
| Squamous | Carcinoma | Cancer | Endocrine | Myeloma/Plasma |
| Neck Cancer | With NUT | | Neoplasia | Cell Neoplasms |
| with Occult | Gene Changes | | Syndromes | |
| Primary | | | | |
| Mycosis | Myelodysplastic | Myelodysplastic/ | Nasal Cavity | Nasopharyngeal |
| Fungoides | Syndromes | Myeloproliferative | and Paranasal | Cancer |
| (Lymphoma) | | Neoplasms | Sinus Cancer | |
| Neuroblastoma | Non-Small Cell | Oral | Oropharyngeal | Ovarian |
| | Lung Cancer | Cancer | Cancer | Cancer |
| Pancreatic | Papillomatosis | Paraganglioma | Paranasal | Parathyroid |
| Cancer | | | Sinus and | Cancer |
| | | | Nasal Cavity | |
| | | | Cancer | |
| Penile | Pharyngeal | Pheochromocytoma | Pituitary | Plasma Cell |
| Cancer | Cancer | | Tumor | Neoplasm/ |
| | | | | Multiple |
| | | | | Myeloma |
| Pleuropulmonary | Primary Central | Primary | Prostate | Rectal |
| Blastoma | Nervous System | Peritoneal | Cancer | Cancer |
| | (CNS) Lymphoma | Cancer | | |
| Recurrent | Retinoblastoma | Rhabdomyosarcoma | Salivary | Sarcoma |
| Cancer | | | Gland | |
| | | | Cancer | |
| Vascular | Uterine | Sézary Syndrome | Small Cell | Small Intestine |
| Tumors | Sarcoma | (Lymphoma) | Lung Cancer | Cancer |
| Soft Tissue | Squamous | Stomach | Throat | Thymoma |
| Sarcoma | Cell | (Gastric) | Cancer | |
| | Carcinoma | Cancer | | |
| Thymic | Thyroid | Transitional Cell | Carcinoma of | Ureter and |
| Carcinoma | Cancer | Cancer of the | Unknown Primary | Renal Pelvis |
| | | Renal Pelvis | | |
| | | and Ureter | | |
| Transitional | Urethral | Uterine | Vaginal | Vulvar |
| Cell Cancer | Cancer | Cancer | Cancer | Cancer |
| Wilms Tumor | | | | |

The disclosed compositions and methods can be used in the treatment of one or more cancers provided in Table 3.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### 2. Immune System Disorders

In some embodiments, the methods administer modified T cells including CAR-CT and/or other CT-fusion protein(s) to treat or prevent one or more immune system disorders, including autoimmune diseases.

Under certain circumstances, the ability of the immune system to distinguish self from foreign antigens can be misdirected against healthy tissues, resulting in the undesirable attack and destruction of normal host cells (i.e., autoimmune diseases). Autoimmune diseases include over 100 types of diseases, with varied etiology and prognoses based on factors such as the affected region, the age of onset, response to the therapeutic agents and clinical manifestation may vary among different people (Muhammad, et al., Chimeric Antigen Receptor Based Therapy as a Potential Approach in Autoimmune Diseases: How Close Are We to the Treatment, Frontiers in Immunology, 11 (2020)).

Auto-antibody-secreting B lymphocytes and self-reactive T-lymphocytes play a key role in the development of autoimmune diseases. Based on the extent of tissue damage, autoimmunity is classified into two general categories, including organ-specific and systemic autoimmune. The former involves a specific area of the body such as type I diabetes (T1D), multiple sclerosis (MS), rheumatoid arthritis (RA), inflammatory bowel diseases (IBDs), and myasthenia gravis (MG), while the latter affects multiple regions of the body, causing systemic lupus erythematosus (SLE) and Sjögren's syndrome (SS). Therefore, in some forms, the methods treat or prevent one or more organ-specific autoimmune diseases in a subject. In other forms, the methods treat or prevent one or more systemic autoimmune diseases in a subject.

In some forms, the methods reduce or prevent one or more physiological processes associated with the development or progression of autoimmune disease in a subject. For example, in some forms, the methods reduce or prevent one or more of epitope spreading, for example, where infections alter the primary epitope into the secondary epitope or form several neoepitopes on antigen-presenting cells; bystander activation or pre-primed autoreactive T cell activation in a T cell receptor (TCR)-independent manner; persistent virus infection, or the constant presence of viral antigens that prompt immune responses; or immunological cross-reactivity between a host and pathogen, for example, due to shared immunologic epitopes or sequence similarities. Non-limiting examples of immune system disorders that can be treated or prevented by the methods include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The disclosed compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

### 3. Other Disease or Disorders

In some forms the methods administer modified T cells including CAR-CT and/or other CT-fusion protein(s) to treat one or more additional disease or disorder in a subject in need thereof. For example, in some forms the methods treat one or more genetic disease or disorders in a subject, such as a hereditary genetic disease or disorder, or a somatic genetic disease or disorder in a subject.

Any of the methods can include treating a subject having an underlying disease or disorder. For example, in some forms, the methods treat a disease or disorder, such as a cancer or auto-immune disease in a patient having another disease or disorder, such as diabetes, a bacterial infection (e.g., Tuberculosis), viral infection (e.g., Hepatitis, HIV, HPV infection, etc.), or a drug-associated disease or disorder. In some forms, the methods treat an immunocompromised subject. In some forms, the methods treat a subject having a disease of the kidney, liver, heart, lung, brain, bladder, reproductive system, bowel/intestines, stomach, bones or skin.

### B. Effective Amounts

In some forms the methods administer modified T cells including CAR-CT and/or other CT-fusion protein(s) in an effective amount. The effective amount or therapeutically effective amount of a pharmaceutical compositions including modified cells, such as therapeutic T cells, can be a dosage sufficient to treat, inhibit, or alleviate one or more symptoms of a disease or disorder, such as a cancer or autoimmune disease, or to otherwise provide a desired pharmacologic and/or physiologic effect, for example, reducing, inhibiting, or reversing one or more of the underlying pathophysiological mechanisms underlying a disease or disorder, such as cancer or autoimmune disease.

In some forms, when administration of the pharmaceutical compositions including modified cells, such as therapeutic T cells, including CAR-CT and/or other CT-fusion protein(s) elicits an anti-cancer response, the amount administered can be expressed as the amount effective to achieve a desired anti-cancer effect in the recipient. For example, in some forms, the amount of the pharmaceutical compositions including modified cells, such as therapeutic T cells, is effective to inhibit the viability or proliferation of cancer cells in the recipient. In some forms, the amount of the pharmaceutical composition including modified cells, such as therapeutic T cells, is effective to reduce the tumor burden in the recipient, or reduce the total number of cancer cells, and combinations thereof. In other forms, the amount of the pharmaceutical compositions including modified cells, such as therapeutic T cells, is effective to reduce one or more symptoms or signs of cancer in a cancer patient, or signs of an autoimmune disease in a patient having an autoimmune disease or disorder. Signs of cancer can include cancer markers, such as PSMA levels in the blood of a patient.

The effective amount of the pharmaceutical compositions including modified cells, such as therapeutic T cells, that is required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions including therapeutic T cells can be determined empirically, and making such determinations is within the skill in the art. In some forms, the dosage ranges for the administration of the compositions including therapeutic T cells are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition including therapeutic T cells used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

It can generally be stated that a pharmaceutical composition containing CAR-CT T cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing CAR-CT expressing cells (e.g., T cells) in the range of about 10⁴ to 10¹² or more cells per square meter of body surface (cells/m).

The infusion can be repeated as often and as many times as the patient can tolerate until the desired response is achieved. CAR-CT T cell compositions can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### C. Modes of Administration

In some embodiments the methods administer modified T cells including CAR-CT and/or other CT-fusion protein(s) in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including modified cells, such as therapeutic T cells, described herein can include, but are not limited to, carriers, thickeners, diluents, buffers, preservatives, surface active agents and the like in addition to the therapeutic(s) of choice.

Pharmaceutical compositions containing one or more modified cells, such as therapeutic T cells including CAR-CT and/or other CT-fusion protein(s), and optionally one or more additional therapeutic agents can be administered to the subject in a number of ways depending on whether local or systemic treatment is desired, and on the area to be treated. Thus, for example, a pharmaceutical composition including modified cells, such as therapeutic T cells, can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions containing one or more genetically modified cells (e.g., CAR-CT T cells) can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### D. Combination Therapy

In some embodiments the methods administer modified T cells including CAR-CT and/or other CT-fusion protein(s) in combination with other therapeutic agents or treatment modalities. Any of the disclosed pharmaceutical compositions including modified cells, such as therapeutic T cells (e.g., containing a population of CAR-CT T-cells), can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

### 1. Additional Anti-Cancer Agents

In some embodiments, the methods administer one or more additional anti-cancer agents to a subject.

In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### 2. Additional Therapeutic Agents Against Autoimmune Diseases

In some embodiments, the methods also include administering one or more conventional therapies for autoimmune diseases to the subject.

Exemplary therapies for autoimmune diseases include immunosuppressive agents, such as steroids or cytostatic drugs, analgesics, non-steroidal anti-inflammatory drugs, glucocorticoids, immunosuppressive and immunomodulatory agents, such as methotrexate, leflunomide, hydroxychloroquine, and sulfasalazine. In some forms, the methods administer one or more disease-modifying antirheumatic drugs (DMARDs). In some forms, the methods administer one or more biologic agents for localized treatment (i.e., agents that do not affect the entire immune system), such as TNF-α inhibitors, belimumab and rituximab depleting B cells, T-cell co-stimulation blocker, anti-interleukin 6 (IL-6), anti-IL-1, and protein kinase inhibitors. In other forms, the methods also administer one or more monoclonal antibodies (mAbs), such as anti-TNFα, anti-CD19, anti-CD20, anti-CD22, and anti-IL6R, or other mAbs that target multiple B cell subtypes, and other aberrant cells in autoimmune diseases.

### IV. Kits

The compositions, reagents, and other materials for cellular genomic engineering can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Provided are kits containing a CT-fusion peptide (e.g., CAR-CT) within a vector (e.g., a viral vector) and/or mRNA encoding the CT-fusion peptide (e.g., CAR-CT), and instructional material for use thereof. In preferred forms, the kit includes a plurality of vectors, where each vector independently contains a CT-fusion peptide (e.g., CAR-CT) for insertion into a host cell genome, such as a CAR expression cassette. In some forms, the kit contains a population of cells (e.g., T cells) collectively containing the CT-fusion peptide (e.g., CAR-CT). The instructional material can include a publication, a recording, a diagram, or any other medium of expression which can be used to communicate the usefulness of the compositions and methods of the kit. For example, the instructional material may provide instructions for methods using the kit components, such as performing transfections, transductions, infections, and conducting screens. In some forms, kits include a transposon that includes a promoter and/or polyadenylation signal operationally linked to a reporter gene and/or a CAR; in some forms, the kit includes a transposon including a CAR that is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof; for example, in some embodiments the CAR targets one or more antigens selected from the group including AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR 3, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE.

In some embodiments, the kit includes a cell or vector including a CAR-CT polypeptide, or other CT-fusion peptide or nucleic acid encoding a CAR-CT, or other CT-fusion peptide. In some embodiments, the CAR-CT is specific for an antigen that is selected from a cancer antigen selected from 4 1BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin; in some forms, the CAR is bispecific or multivalent; in some forms, the CAR is anti CD19 or anti CD22, or both. Exemplary CARs include CD19BBz or CD22BBz. In exemplary embodiments, the kits include a nucleic acid and/or a vector expressing or encoding the CAR-CT and/or cells. Exemplary cells include a T cell, hematopoietic stem cell (HSC), macrophage, natural killer cell (NK), or dendritic cell (DC). In some embodiments, the T cell is a CD8+ T cell selected from effector T cells, memory T cells, central memory T cells, and effector memory T cells. In some embodiments, the T cell is a CD4+ T cell selected from Th1 cells, Th2 cells, Th17 cells, and Treg cells.

The disclosed compositions and methods can be further understood through the following numbered paragraphs.

1. A polypeptide including
(a) an amino acid sequence from the cytosolic domain of CTLA-4, including between 25 and 41 contiguous amino acids of SEQ ID NO:3, or a functional fragment or variant thereof; and
(b) a heterologous amino acid sequence that is heterologous to CTLA-4.

2. A polypeptide including
(a) an amino acid sequence including at least 70% and less than 100% sequence identity to SEQ ID NO:3 or functional fragment thereof;
and
(b) optionally a heterologous amino acid sequence that is heterologous to CTLA4.

3. The polypeptide of paragraph 1 or 2, further including the amino acid sequence of SEQ ID NO:5, or a functional fragment or variant thereof.

4. The polypeptide of paragraph 1 or 3, including the amino acid sequence of SEQ ID NO:7 or a functional fragment or variant thereof.

5. The polypeptide of paragraph 3, further including one or more additional copies of the amino acid sequence of SEQ ID NO:5, or a functional fragment or variant thereof.

6. The polypeptide of any one of paragraphs 1 or 3-5, including the amino acid sequence of SEQ ID NO:51 or a functional fragment or variant thereof.

7. The polypeptide of paragraph 1, wherein the amino acid sequence from the cytosolic domain of CTLA-4, is SEQ ID NO:3, or SEQ ID NO:7.

8. The polypeptide of paragraph 7, wherein the polypeptide further includes any one of SEQ ID NOs 5, or 9-50.

9. The polypeptide of any one of paragraphs 1-8, wherein the polypeptide can interact with clathrin adaptor activating protein 2 (AP-2), optionally wherein interaction includes the ability to co-immunoprecipitate.

10. The polypeptide of any one of paragraphs 1-9, including two YVKM amino acid motif(s).

11. The polypeptide of any one of paragraphs 1-10, wherein the heterologous sequence includes one or more of a chimeric antigen receptor (CAR), programmed death protein 1 (PD1), protein transduction domain, fusogenic polypeptide, targeting signal, expression and/or purification tag.

12. The polypeptide of paragraph 11, wherein the heterologous sequence includes a chimeric antigen receptor (CAR), and wherein the polypeptide is present within the intracellular region of the CAR.

13. The polypeptide of paragraph 11 or 12, wherein the heterologous sequence includes a chimeric antigen receptor (CAR), and wherein the polypeptide is contiguous with the carboxyl terminus of the CAR.

14. The polypeptide of paragraph 11, or 12, or 13 wherein the heterologous sequence includes a chimeric antigen receptor (CAR) including an intracellular component of CD3 zeta, and wherein the polypeptide is contiguous with the intracellular component of CD3 zeta.

15. The polypeptide of any one of paragraphs 11-14, wherein the CAR is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

16. The polypeptide of paragraph 15, wherein the CAR targets one or more antigens selected from AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, 1gK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR R, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE.

17. The polypeptide of paragraph 15, wherein the antigen is a cancer antigen selected from 4 1BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5ρ1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin.

18. The polypeptide of paragraph 17, wherein the CAR is anti CD19 or anti CD22, or both, optionally wherein the CAR is CD19BBz-CAR or CD22(m971)-CAR.

19. The polypeptide of any one of paragraphs 1-6, wherein the heterologous sequence comprises the amino acid sequence of PD1, optionally wherein the polypeptide comprises the amino acid sequence of any one of SEQ ID NOS: 74, 76, or 126.

20. A nucleic acid including a nucleic acid encoding the polypeptide of any one of paragraphs 1-19.

21. A nucleic acid including a nucleic acid encoding a polypeptide including a chimeric antigen receptor (CAR) and one or more of SEQ ID NO:3, or SEQ ID NO: 7, or SEQ ID NO:51.

22. The nucleic acid of any one of paragraphs 20-21, wherein the nucleic acid is RNA or DNA.

23. The nucleic acid of any one of paragraphs 20-22, wherein the nucleic acid is mRNA.

24. The nucleic acid of any one of paragraphs 20-23, wherein the nucleic acid includes an expression control sequence(s).

25. The nucleic acid of any one of paragraphs 20-24, wherein the nucleic acid is, or is encoded by a vector or a transposon.

26. The nucleic acid of paragraph 25, wherein the vector is a viral vector.

27. The nucleic acid of paragraph 26, wherein the viral vector is selected from a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector including a combination of any two or more of a Adeno-associated virus (AAV) vector, Herpes Simplex virus (HSV) vector, vesicular stomatitis (VSV) vector, or a human Bocavirus vector (hBoV).

28. The nucleic acid of paragraph 25, wherein the vector is a nucleic acid expression vector selected from a plasmid, a cosmid, and a replicon.

29. The nucleic acid of any one of paragraphs 20-28, wherein the nucleic acid includes a promotor.

30. The nucleic acid of any one of paragraphs 20-29, including one or more of a protein transduction domain, fusogenic polypeptide, or targeting signal conjugated thereto.

31. An isolated cell including the polypeptide of any one of paragraphs 1-19, or the nucleic acid of any one of paragraphs 20-30.

32. The isolated cell of paragraph 31, wherein the cell is a T cell, hematopoietic stem cell (HSC), macrophage, natural killer cell (NK), or dendritic cell (DC).

33. The isolated cell of paragraph 32, wherein the T cell is a CD8+ T cell selected from effector T cells, memory T cells, central memory T cells, and effector memory T cells.

34. The isolated cell of paragraph 32, wherein the T cell is a CD4+ T cell selected from Th1 cells, Th2 cells, Th17 cells, and Treg cells.

35. The isolated cell of any one of paragraphs 31-34, wherein the polypeptide includes
(i) an amino acid sequence encoding a CAR; and
(ii) an amino acid sequence of one or more of SEQ ID NO:3, or SEQ ID NO: 7,
or SEQ ID NO:51, or a variant having at least 75% identity to SEQ ID NO:3, or at least 75% identity to SEQ ID NO: 7, or at least 75% identity to SEQ ID NO:51.

36. The isolated cell of any one of paragraphs 31-35, wherein the polypeptide includes
(i) an amino acid sequence encoding a CAR; and
(ii) an amino acid sequence of SEQ ID NO: 7, or a variant having at least 75% identity to SEQ ID NO: 7.

37. The isolated cell of paragraph 36, wherein the amino acid sequence of SEQ ID NO: 7 is contiguous with the residue at the carboxyl terminus of the CAR.

38. A population of cells derived by expanding the cell of any one of paragraphs 31-37.

39. A pharmaceutical composition including the population of cells of paragraph 38 and a pharmaceutically acceptable buffer, carrier, diluent or excipient.

40. A method of treating a subject having a disease, disorder, or condition including administering to the subject an effective amount of the pharmaceutical composition of paragraph 39.

41. A method of treating a subject having a disease, disorder, or condition associated with an elevated expression or specific expression of an antigen, the method including administering to the subject an effective amount of a cell of paragraph 35 or 36, wherein the CAR targets the antigen, optionally wherein the cell is a T cell, optionally a CD8+ T cell.

42. The method of any one of paragraphs 40-41, wherein the cell was isolated from the subject having the disease, disorder, or condition prior to the introduction to the cell.

43. The method of any one of paragraphs 40-41, wherein the cell was isolated from a healthy donor.

44. The method of any one of paragraphs 40-43, wherein the subject is a human.

45. The method of any one of paragraphs 40-44, wherein the subject has a disease selected from cancer, an inflammatory disease, a neuronal disorder, HIV/AIDS, a diabetes, a cardiovascular disease, an infectious disease (including a viral, a protozoan, a bacterial disease, and an allergy), an autoimmune disease and an autoimmune disease, and a genetic disorder.

46. The method of paragraph 45, wherein the disease is a cancer.

47. A method of introducing a CT fusion peptide into a cell, the method including introducing to the cell:
(i) a vector or transposon or mRNA encoding a polypeptide of any one of paragraph 1-19; and
(ii) causing the polypeptide to be expressed in the cell.

48. A chimeric antigen receptor (CAR) including a CT polypeptide (CAR-CT), wherein the CAR-CT targets CD22 and includes the amino acid sequence of any one of SEQ ID NOs:55, 57, 59.

49. A chimeric antigen receptor (CAR) including a CT polypeptide (CAR-CT), wherein the CAR-CT targets CD22 and includes the amino acid sequence of SEQ ID NO:57.

50. A chimeric antigen receptor (CAR) including a CT polypeptide (CAR-CT), wherein the CAR-CT targets CD19 and includes the amino acid sequence of any one of SEQ ID NOs: 61 63, 65.

51. A chimeric antigen receptor (CAR) including a CT polypeptide (CAR-CT), wherein the CAR-CT targets CD19 and includes the amino acid sequence of SEQ ID NO:63.

52. A nucleic acid, including a nucleic acid sequence encoding the chimeric antigen receptor of any one of paragraphs 48-51.

53. The nucleic acid of paragraph 52, wherein the nucleic acid is a vector or a transposon.

54. An isolated cell including the CAR of any one of paragraphs 48-51, or the nucleic acid of any one of paragraphs 52-53.

55. A population of cells derived by expanding the cell of paragraph 54.

56. A pharmaceutical composition including the population of cells of paragraph 55 and a pharmaceutically acceptable buffer, carrier, diluent or excipient.

57. A method of treating a subject having a disease, disorder, or condition including administering to the subject an effective amount of the pharmaceutical composition of paragraph 56.

58. The method of paragraph 57, wherein the disease is a cancer.

59. The method of paragraphs 46 or 58, wherein the cancer is a leukemia.

60. A composition, formulation, or method as described herein including, but not limited to, the text and figures

### EXAMPLES

### Example 1: Cytoplasmic tail of CTLA-4 (CT) enables quantitative control of receptor-mediated trogocytosis

### Methods

### Vector Construction

All lentivirus plasmids were generated by inserting target coding sequences into lentivirus transfer plasmid backbone (Addgene, #75112). Specifically, the pLenti-EFS-Flag-scFV(m971)-CD28-41BB-CD3zeta-T2A-mScarlet (CD22-CAR) was generated by amplifying the sequence from a gblock synthesized by IDT, and inserting it into the lentiviral backbone using Gibson Assembly. Afterwards, to generate CAR-CCT constructs, 1CCT, 2CCT and 3CCT were inserted right before the T2A sequences, by amplifying 1-3 CCTs from the synthesized sequences with three tandem CCTs. CD19-CAR and CD19-CAR-CCT constructs were generated by replacing the m971 scFv with the FMC63 scFv from above CD22-CAR constructs. Similar methods were used to generatepLenti-EFS-CD22-GGGGS-mTagBFP and pLenti-EFS-CD22-GGGGS-eGFP constructs each of which include a nucleic acid sequence encoding the peptide linker (GGGGS (SEQ ID NO:125)).

### Cell Culture

293T, NALM6 and Jurkat cells were purchased from ATCC. Human PBMCs were purchased from Stemcell. NALM6 expressing GFP-luciferase (NALM6GL) was previously generated in lab (Dai et al., Nat Methods 16, 247-254 (2019)). 293T cells were cultured in DMEM (Gibco) media supplemented with 10% FBS (CORNING) and 200 U/mL penicillin-streptomycin (Gibco), hereafter referred to as cDMEM. NALM6 cells and Jurkat cells were cultured in RPMI-1640 (Gibco) media supplemented with 10% FBS and 200 U/mL penicillin-streptomycin, hereafter referred to as cRPMI. Human PBMCs were cultured in X-VIVO™ 15 media (Lonza) supplied with 5% human AB serum (MP Biomedical) and 10 ng/mL human IL-2 (Peprotech), hereafter referred to as cX-VIVO. All cells were grown at 37° C. and 5% CO2 with saturating humidity.

### Lentivirus Production

One day before transfection, 40 million 293T cells (passage number less than 20) were seeded into a 150 mm dish. The next day, old medium was replaced with pre-warmed fresh cDMEM. For each dish, 20 μg transfer plasmid, 10 μg packaging plasmid psPAX2 (Addgene) and 5 μg envelop plasmid pMD2.G (Addgene) were diluted with 1 mL plain DMEM. In a separate tube, 87.5 μl LipoD293 was diluted with 1 mL plain DMEM and then added to the diluted DNA mixture. This transfection mix was then briefly vortexed and incubated at room temperature for 15 minutes before being added to the 293T cells. Lentivirus was harvested 48 hours post transfection by collecting the supernatant of transfected cells. The supernatant was then spun down at 3,000 g for 15 minutes to get rid of cell debris. Lentivirus was concentrated by adding 40% (w/v) PEG8000 directly to the supernatant to final concentration of 8% (w/v) PEG8000, and then incubated at 4° C. overnight. The next day, the lentivirus was spun down at 1,500 g for 30 minutes, pegylated viral pellet was resuspended with 1 mL fresh cRPMI or cX-VIVO medium. Virus was then stored at −80° C. before usage.

### Generation of Stable Cell Lines

All stable cell lines used in this study were generated by lentiviral infection. Briefly Jurkat, NALM6 or NALM6GL cells were spin-infected with lentiviruses carrying corresponding constructs at 2,000 rpm (−900 g), 37° C. for 2 hours. Infected cells were then purified based on reporter gene (GFP, BFP or mScarlet) expression by flow cytometer. NALM6-CD22-GFP cells were generated through transduction with a lentiviral vector that expressed full-length human CD22 and eGFP linked by a GGGGS (SEQ ID NO:125) linker. NALM6GL-CD22-BFP cells were generated using a similar lentiviral construct, where eGFP was swapped for mTagBFP in NALM6GL cells. CAR-Jurkat cells were generated by transducing Jurkat cells using the same lentiviral constructs showed in FIG. 9A..

### NALM6 In Vitro Trogocytosis Assay

0.1 million NALM6 receiver cells expressing fPD1, t-PD1, PD1-CT, PD1-2CT or PD1-3CT were cocultured with 0.1 million NALM6GL-PDL1-BFP giver cells at E/T=1/1 in 96-well U bottom plate for 24 hours. In some experiments as indicated in the figure legend, anti-human PD1 antibody (10 μg/mL), anti-human PDL1 antibody (10 μg/mL) or isotype control antibody (10 μg/mL) was supplemented at the beginning of the assay. Trogocytosis was quantified by measuring the BFP signal transfer onto the receiver cells.

### Antibody and Flow Cytometry

All antibodies used in this study are listed in the Key resources table. Cell surface antigens were stained with indicated antibody cocktails in MACS buffer on ice for 15 minutes or 1 hour as indicated in the figure legend. Cycling receptors were stained with indicated antibody cocktails in cRPMI at 37° C. for 1 hour. PDL1 binding ability of chimeric PD1 was tested by first staining NALM6 receiver cells with recombinant human PDL1-Fc (R&D) in MACS buffer on ice for 15 minutes, and then with BV421 anti-human Fc (Biolegend) in MACS buffer on ice for another 15 minutes. LIVE/DEAD Fixable Near-IR Dead Cell Stain (Invitrogen) was included for all staining to exclude unspecific staining of dead cells. Samples were collected or sorted on 4 lasers (405 nm, 488 nm, 561 nm and 640 nm) Aria II cell sorter (BD). Sorting purity was checked after every sort to make certain it was higher than 95%. The data were analyzed using FlowJo software 10.8.0 (BD).

### Slides Staining and Confocal Microscope

About 5000 Purified receiver cells that had performed trogocytosis (mScarlet+BFP+) were seeded onto poly-D-lysine treated culture slide (Corning) and incubated at 37° C. for 1 hour. Cells were then stained with deep red plasma membrane stain (ThermoFisher) at 37° C. for 5 minutes according to the manufacturer's instruction. Fluoromount-G™ mounting medium (ThermoFisher) was used to seal the slide before being imaged by Nikon Spinning Disk Confocal Microscope with the 40× objective. Image analysis was performed using ImageJ.

### Human T Cell Isolation and Culture

Human CD3 T cells were isolated directly from PBMC samples by using magnetic positive selection. Briefly, 15 million PBMCs were labeled with anti-human CD3-biotin (Biolegend, 1:100) in MACS buffer (PBS supplemented with 0.5% BSA and 2 mM EDTA) at 4° C. for 10 minutes. After being washed with MACS buffer, PBMCs were resuspended with 120 μL MACS buffer and then stained with 30 μL of anti-biotin beads (Miltenyi) at 4° C. for 15 minutes. After a final wash with MACS buffer, labeled CD3 T cells were separated by using MACS LS Columns (Miltenyi) and MACS Separators (Miltenyi) according to the manufacturer's instructions. Purified CD3 T cells were cultured in cX-VIVO medium and stimulated with Dynabeads® Human T-Activator CD3/CD28 (Thermo) at a bead-to-cell ratio of 1:1 for 24 hours before being infected with lentiviruses.

Human T Cell Transduction with Lentivirus

Human CD3 T cells activated for 24 hours were collected, and resuspended with concentrated lentiviruses, supplemented with 8 μg/mL polybrene, at a concentration of 1 million/mL. Cells were plated in 24-well plate and spun at 2,000 rpm (˜900 g) for 2 hours at 37° C. Immediately after the spin, the virus supernatant was aspirated and replaced with fresh cX-VIVO medium. Cells were split 1 to 2 every other day and transgene expression was checked 4 days after infection by flow cytometry. CAR-T cells were sorted on day 7 based on mScarlet expression and expanded in vitro for another 7 days before being used for in vitro and in vivo experiments.

### Antibody and Staining for Flow Cytometry

All antibodies used in this study are listed in the Antibodies section of the Nature Portfolio Reporting Summary. Cell surface antigens were stained with indicated antibody cocktails in MACS buffer on ice for 15 minutes as indicated in the figure legend. To stain endocytic CAR or cycling CTLA-4, CAR-T cells were stained using anti-flag-BV421(Rat-IgG2a, Biolegend) or anti-CTLA-4-APC (Mouse IgG2a, Biolegend) at 37□C for 1 hour, followed by washing with ice old MACS buffer. Those cells were then stained with anti-Rat-IgG2a-Alexaflour647 (Biolegend) or anti-mouse IgG2a-BV421(Biolegend) at 4° C. for 15 minutes before running on the flow cytometer. To quantify apoptosis, cells were stained with Annexin V Pacific-blue conjugates (ThermoFisher) in Annexin V binding buffer (BD) at room temperature for 15 minutes, cells were then immediately analyzed by flow cytometer. LIVE/DEAD Fixable Near-IR Dead Cell Stain (Invitrogen) was included for all staining to exclude unspecific staining of dead cells. Samples were collected or sorted on a 4-laser (405 nm, 488 nm, 561 nm and 640 nm) Aria II cell sorter (BD). For sorting experiments, sorting purity was checked after every sort to make certain it was higher than 95%. All flow cytometry data were analyzed using FlowJo software 10.8.0 (BD).

### In Vitro CAR T Killing Assay

Variable amounts of purified CAR T cells were cultured with fixed amount of NALM6GL cells at an E/T ratio of 1/2, 1/4 or 1/8 in a 96-well U bottom plate. 24 hours later, cells were either collected for flow cytometry analysis or re-stimulated with the same amount of NALM6GL cells as used in the first round coculture. 24 or 48 hours later (indicated in the figure legend), NALM6GL cells and CAR-T cells were stained with indicated antibody cocktails. Cell counting beads (Biolegend) were added into all samples for absolute cell number measurement. For multiplex cytokine detection, the supernatant from cocultures was collected and measured by LEGENDplex™ Human CD8/NK Panel (Biolegend) according to the manufacturer's instructions.

### In Vivo Evaluation of CAR-T Efficacy

NOD.Cg-Prkdcscid Il2rgtm1Wjl/SzJ (NSG) mice were purchased from the Jackson Laboratory and bred in-house. NSG mice (female, 6-10 weeks old) were inoculated with 0.5 million NALM6GL cells intravenously (i.v.) on day 0 and treated with 1 million CAR-T cells (i.v.) on day 4. Relapse was modeled by re-challenging all mice with 0.5 million NALM6GL cells (i.v.) on day 12. To better model the clinical response to CAR-T cells in NSG mice (Jespersen, et al., Nat Commun 8, 707 (2017)), 2.5 μg human recombinant IL2 (Peprotech) was administered subcutaneously (s.c.) everyday starting on day 4 for 24 days. Disease progression was monitored by bioluminescence imaging and survival analysis. For the in vivo phenotyping of CAR-T cells, 2.5 μg human recombinant IL2 was administered s.c. every day from day 4 to day 14. All mice were sacrificed on day 15. Spleen and bone marrow tissues were collected and processed into single cell suspension. Briefly, spleens were prepared by mashing them through 100 μm filters. For bone marrow isolation, femur with both ends cut by scissors was isolated, and bone marrow was flushed out with 2 mL cRPMI using a 25 G needle. For both spleen and bone marrow suspensions, red blood cells were lysed with ACK Lysis Buffer (Lonza), incubated for 2 minutes at room temperature, and washed with cRPMI. Lymphocytes were poured through a 40 μm filter before staining with antibody cocktail. Cell counting beads (Biolegend) were added into all samples for absolute cell number measurement.

### Proliferation Assay

Purified CAR-T cells were stained in PBS with 10 μM Cell Proliferation Dye eFluor™ 450 (ThermoFisher) at 37° C. for 5 minutes. Cells were then washed three times with cRPMI medium and resuspended in cX-VIVO medium at a final concentration of 0.5 million/mL. 200 μL cells were seeded into a 96-well U bottom plate. 4 days later, eFluor450 dilution was measured by flow cytometry.

### Fratricide Assay

CAR, CAR-1CT, CAR-2CT and CAR-3CT cells stained with 1 μM eflour450 were mixed with CAR-T cells stained with 10 μM eflour450 at 1:1 ratio. These cells were then cultured with or without NALM6GL stimulation at an E/T ratio of 1:2 for 24 hours. Flow cytometry was used to determine the relative percentage of eflour450low and eflour450high population. Resistance to fratricide was indicated by relative survival of CAR T cells. And it was calculated by following equation: Relative survival (%)=[(lw:hw)−(lwo:hwo)]/(lwo:hwo)*100%, in which lw and hw stands for percentage of eflour450 low and high population, respectively, with NALM6GL cells stimulation, while lwo and hwo stands for percentage of eflour450 low and high population, respectively, without NALM6GL cells stimulation.

### Measurement of Phosphorylation and Cytokine Production

To measure phospho-ERK, CAR-T cells were first starved overnight in serum free X-VIVO medium, and stained for LIVE/DEAD Fixable Near-IR Dead Cell Stain (Invitrogen). After washed with ice old PBS, those CAR-T cells were incubated with 2 g/mL biotinylated human CD22 (ECD) on ice for 30 minutes. Signal transduction was induced by crosslinking with 10 μg/mL streptavidin (Biolegend) at 37° C. for 5 minutes. Crosslinking was stopped immediately with the Phosflow™ Fix Buffer I (BD) and fixed at 37° C. for 10 minutes. After this, cells were permeabilized with BD Phosflow™ Perm Buffer III on ice for 30 minutes, followed by Anti-ERK1/2 (pT202/pY204)-Alexaflour647 (Biolegend) staining at room temperature for 30 minutes. For the intracellular IFNg and TNFa staining, CAR-T cells were cocultured with NALM6GL cells at indicate E/T ratio for 4 hours with the presence of 1× Brefeldin A (Biolegend). Cells were first stained with the Live/Dead-NearIR dye before fixed, permeabilized and stained with BD Cytofix/Cytoperm™ Buffer System according to manufacturer's suggestion.

### Measurement of Recycling CAR

The method used for quantification of recycling receptors over time has been described by others (Janman, et al., Immunology 164, 106-119 (2021)). Briefly, CAR-T cells were first stained with anti-Flag (Biolegend) at 37° C. for 1 hour, and washed with ice cold MACS buffer. The anti-Rat IgG2a-Alexaflour647 secondary antibody either stained at 4° C. for 15 minutes (baseline) or at 37° C. for 30 minutes or 60 minutes to see an increase in the secondary antibody signal when compared to the baseline. The increase in the percentage of cells stained positive for the secondary antibody was quantified by flow cytometry as an indication of recycling.

### Measurement of Recycling CAR or CTLA-4

Briefly, CAR-T cells were first stained with anti-Flag antibody (Biolegend) or anti-CTLA-4 antibody (Biolegend) at 37° C. for 1 hour and washed with ice cold MACS buffer. The secondary antibody either stained at 4° C. for 15 minutes (baseline) or at 37° C. for 30 minutes or 60 minutes in order to see an increase in the secondary antibody signal when compared to the baseline. The increase (fold change) in the percentage of cells stained positive for the secondary antibody was quantified by flow cytometry as an indication of recycling.

### Cycloheximide Chase Assay

The stability of CAR was quantified by treating CAR-T cells with 50 μg/mL cycloheximide (Sigma) at 37° C. for up to 4 hours. Total CAR expression was quantified by staining intracellular flag expression using BD Cytofix/Cytoperm™ Buffer System according to manufacturer's suggestion.

CAR Degradation with Antibody Feeding

Briefly, CAR-T cells were incubated with anti-Flag antibody (Biolegend) at 37° C. for 1 hour. Cells were then washed and incubated at 37° C. for up to 4 hours with or without 10 μM bortezomib (Sigma) or 10 nM BafA1. Cells were then fixed and permeabilized with BD Cytofix/Cytoperm™ Buffer System, and stained with Anti-Rat-IgG2a-Alexaflour647 before analyzed by flow cytometry.

### Quantification of CD22 Transfer by Confocal Microscopy

About 10,000 Purified CAR-T cells that had performed trogocytosis (mScarlet⁺ BFP⁺) were seeded onto μ-Slide 8-Well glass bottom chamber (Ibidi) that had been previously treated with poly-L-lysine. Cells were then stained with deep red CellMask™ Plasma Membrane dye (Thermistor) at 37° C. for 5 minutes according to the manufacturer's instruction. Cells were imaged by Leica Stellaris 8 FALCON with the 40× objective (oil). Representative images were exported using ImageJ (https://imagej.nih.gov/). The percentage of CD22 membrane colocalization was analyzed by a customized MATLAB (Mathworks, Natick, MA) script. Specifically, Images of BFP tagged CD22 antigen and CellMask stained membrane were first binarized with a global threshold for pixel intensity (10 for antigen and 15 for membrane) to classify each pixel into either foreground pixel or background pixel. Cells appear as a ring in membrane mask and all closed rings were filled to generate a binarized image where foreground pixels represent intact cells (cell mask). Cytosol mask was obtained by subtracting membrane mask from cell mask. Antigen mask then underwent a size filtering to remove objects with too few (<3 pixels) or too many pixels (>300 pixels) which mainly originates from impulse noise and dust. Cell mask underwent a similar size filtering (<100 pixels and >1000 pixels) to remove objects originating from impulse noise, broken cells and clusters of cells such that the filtered cell mask only contains well isolated single cells. Each single cell in the filtered cell mask was then discarded/selected for final colocalization calculation based on two quality control indexes: 1) cytosol percentage; 2) number of contained antigen pixels. Too low cytosol percentage (<40%) might be caused by endocytosis of the membrane staining dye into cells and can lead to unreliable membrane segmentation. Therefore, cells with too low cytosol percentage were excluded from the analysis. Too few antigen pixels (<20 pixels) lead to significant uncertainty in estimation of colocalization percentage and thus were also excluded from further analysis. Finally, for all the remaining cells, antigen-membrane colocalization percentage was calculated by the following formula where I_{antigen-cytosol}, I_{antigen-cell} stands for intensity of each pixel classified as foreground in both antigen and cytosol mask, respectively.

I_{antigenbackground} stands for mean intensity of background pixels in antigen image (2.5 used for all analysis)

$${p}_{colocalization} = 1 - \frac{\sum ( {I}_{a n t i gen - cytosol )} - {I}_{a n t igen background} )}{\sum ( {I}_{a n t i gen - cell} - {I}_{antigen background} )}$$

### Time-Lapse Live Cell Imaging of Trogocytosis and Fratricide

40,000 CAR-T cells and NALM6GL-CD22-BFP cells were seeded at an E to T ratio of 2:1 into a μ-Slide 8-Well glass bottom chamber (Ibidi) that had been previously treated with 7.5 μg/cm² poly-L-lysine (Sigma). 5 nM SYTOX™ Deep Red Nucleic Acid Stain (ThermoFisher) was supplemented into the culture to allow for dead cell staining. Cells were imaged with Leica Stellaris 8 FALCON with the 40× objective (oil) in the live cell imaging chamber at 37° C. and 5% CO₂. A 4×4 field-of-view was taken at a scan interval of every 3 to 5 minutes for up to 8 hours. Images were processed and exported through ImageJ (imagej.nih.gov/).

### Trogocytosis Assay

CAR-T or CAR-Jurkat cells were coculture with NALM6GL, NALM6GL-CD22-BFP or NALM6-CD22-GFP cells for indicated time at a fixed E to T ratio, as specified in the figure legend. Trogocytosis of CD22 by CAR-T cells were quantified by measuring CD22 using antibody, BFP or GFP signal. For trogocytosis inhibition, as indicated in the figure legend, CAR T cells were pre-treated with 1 μM latrunculin A (Sigma-Aldrich) at 37° C. for 15 min before co-incubation with target cells.

### Degranulation Assay

CAR-Jurkat cells were cocultured with NALM6-CD22-GFP at an E/T ratio of 1:1 for 4 hours to allow for CD22-GFP transfer from NALM6 to CAR-Jurkat cells. Both Trog⁺ CAR-Jurakt and Trog⁻ CAR-Jurkat cells were sorted out based on their expression of CD22-GFP using an AriaII sorter (BD). Those cells were then used as target cells and cocultured for 2 hours with fresh CAR-T cells that had been labeled with 10 nM eFluor450 to allow for differentiation between CAR-T and CAR-Jurkat cells. Variable CAR-T to CAR-Jurkat ratios were used as indicated in the figure legend. Anti-CD107-APC (Biolegend) and 1× Monensin (Biolegend) were added into the culture during the incubation. Cells were then stained with LIVE/DEAD Fixable Near-IR Dead Cell Stain (Invitrogen) before analyzed by flow cytometry.

### Measurement of Relative CAR-T Survival

CAR, CAR-1CCT, CAR-2CCT and CAR-3CCT cells stained with 1 μM eFluor450 were mixed with CAR-T cells stained with 10 μM eFluor450 at 1:1 ratio. For in vitro measurement, these mixed cells were then cultured with or without NALM6GL stimulation at an E/T ratio of 1:2 for 24 hours. For in vivo measurement, NSG mice (female, 6-10 weeks old) were first inoculated with 1 million NALM6GL cells intravenously (i.v.) on day 0. Two million of these labeled cell mixture was transferred to the leukemia NSG models on day 4. One day post CAR-T transfer, bone marrow samples were collected and processed into single cell suspension according to methods mentioned above. Cells were stained using LIVE/DEAD Fixable Near-IR Dead Cell Stain (Invitrogen) and anti-human CD22 APC (Biolegend) before being analyzed by flow cytometer. Flow cytometry was used to determine the relative percentage of eFluor4501^{low} and eFluor450^{high} population. Relative survival of CAR T cells. And it was calculated by following equation: Relative survival (%)=[(l_{w}:h_{w})−(l_{w o}:h_{wo})]/(l_{wo}:h_{wo})*100%, in which 1, and h, stands for percentage of eFluor450 low and high population, respectively, with NALM6GL cells stimulation (in vitro assays) or after transfer (in vivo assays), while l_{wo} and h_{wo} stands for percentage of eFluor450 low and high population, respectively, without NALM6GL cells stimulation (in vitro assays) or before transfer (in vivo assays).

### In Vivo Evaluation of CAR-T Performance

NOD.Cg-Prkdc^{scid} Il2rg^{tmlWjl}/SzJ (NSG) mice were purchased from the Jackson Laboratory and bred in house. NSG mice (female, 6-10 weeks old) were inoculated with 0.5 million NALM6GL cells intravenously (i.v.) on day 0 and treated with 1 million CAR-T cells (i.v.) on day 4. Relapse was modeled by re-challenging all mice with 0.5 million NALM6GL cells(i.v.) on day 12. To better model the clinical response to CAR-T cells in NSG mice (Jespersen, et al., Nat Commun 8, 707, (2017)), 2.5 μg human recombinant IL2 (Peprotech) was administered subcutaneously (s.c.) everyday starting on day 4 for 24 days. Disease progression was monitored by bioluminescence imaging and survival analysis. For the in vivo phenotyping of CAR-T cells, 2.5 μg human recombinant IL2 was administered s.c. every day from day 4 to day 14. All mice were sacrificed on day 15. Spleen and bone marrow tissues were collected and processed into single cell suspension. For preparations, spleens were prepared by mashing them through 100 um filters. For bone marrow isolation, femur with both ends cut by scissors was isolated, and bone marrow was flushed out with 2 mL cRPMI using a 25 G needle. For both spleen and bone marrow suspensions, red blood cells were lysed with ACK Lysis Buffer (Lonza), incubated for 2 minutes at room temperature, and washed with cRPMI. Lymphocytes were poured through a 40 μm filter before staining with antibody cocktail. Cell counting beads (Biolegend) were added to all samples for absolute cell number measurement by Aria II cell sorter (BD).

scRNA-Seq Library Preparation and Sequencing

Live CAR-T cells were sorted based on mScarlet expression from both the spleen and bone marrow samples processed as described above. For all four groups (CAR, CAR-1CCT, CAR-2CCT and CAR-3CCT), samples from 3 individual mice within the same group were pooled together to minimize sampling bias. Sorted cells were washed with PBS, and cell number and viability were assessed by trypan blue (Lonza) staining. About 2,000 to 10,000 purified mScarlet⁺ cells were used for scRNA-seq library preparation using Chromium Next GEM Single Cell 5′ Reagent Kits V2 (10× Genomics) according to manufacturer's instructions. The single cell libraries were sequenced by NovaSeq 6,000 (Illumina) with 2×150 read length.

scRNA-Seq Data Analysis

Analysis of scRNA-seq was performed using standard pipelines with custom codes. In brief, raw FASTQ data were pre-processed with Cell Ranger v6.0.1. The processed data matrices were then analyzed and visualized using the Seurat v4 package (Satija, et al., Nat Biotechnol 33, 495-5020 (2015)). More specifically, the dataset was filtered to retain cells with <10% mitochondrial counts and 200-2,500 unique expressed features. The dataset was then log-normalized, scaled using the reciprocal-PCA dimensional reduction with 2000 anchors (Stuart, et al., Cell, 177, 1888-1902 (2019)). And dimensional reduction was performed by uniform manifold approximation and projection (UMAP) (Becht, et al., Nat Biotechnol, doi:10.1038/nbt.4314 (2018)). using the first 10 dimensions from PCA, which were chosen by the inflection point of an elbow plot. Cells were clustered in low-dimensional space by generating a shared nearest neighbor (SNN) graph (k=20, first 10 PCs). Each cell cluster was annotated by specific type of T cells using canonical marker genes. An empirical resolution (0.2), followed by a second-step resolution for sub-clustering, was chosen for better separation of CD4 and CD8 T cell populations. Bulk RNA sequencing and data processing

To profile baseline differences, purified CAR-T cells expanded in vitro for 14 days were collected. To profile differences under repeated stimulations, CAR-T cells expanded in vitro for 14 days were cocultured with NALM6GL cells at E/T=1/2, followed by a second round of NALM6GL cells stimulation 24 hours after the initial coculture. mScarlet+ CAR-T cells were then sorted at 72 hrs after the initial coculture. These purified CAR-T cells were lysed for bulk RNA extraction using RNeasy Plus mini isolation kit (Qiagen). Library preparations were performed using a NEBNext® Ultra™ RNA Library Prep Kit for Illumina according to manufacturer's instruction. Samples were barcoded and multiplexed using NEBNext® Multiplex Oligos for Illumina® (Index Primers Set 1). Libraries were then sequenced with Novaseq (Illumina). Fastq files generated by sequencing were aligned and mapped using STAR (Dobin, et al., Bioinformatics 29, 15-21 (2013)) with the human genome assembly version GRCh38. Differential expression was analyzed using DEseq2 (Love, et al., Genome Biol. 15, 550 (2014)). p (adj)<0.01 and |log2FoldChange|>1 were set as the cutoff for determining differentially expressed genes, which were selected for over representation analysis (ORA) using R package enrichplot (G. Yu, R package version 1.8.1. https://github.com/GuangchuangYu/enrichplot, (2020)). Visualizations of differentially expressed genes, such as volcano plots and heatmaps, were generated using standard R packages such as pheatmap and VennDiagram.

### Statistics

All statistical methods are described in figure legends. The P values and statistical significance were estimated for all analyses. For example, One-way ANOVA, two-way ANOVA, Dunnett's multiple comparisons test or Tukey's multiple comparisons test was used to compare multiple groups. Different levels of statistical significance were accessed based on specific p values and type I error cutoffs (0.05, 0.01, 0.001, 0.0001). Data analysis was performed by GraphPad Prism v.8. and RStudio. Results

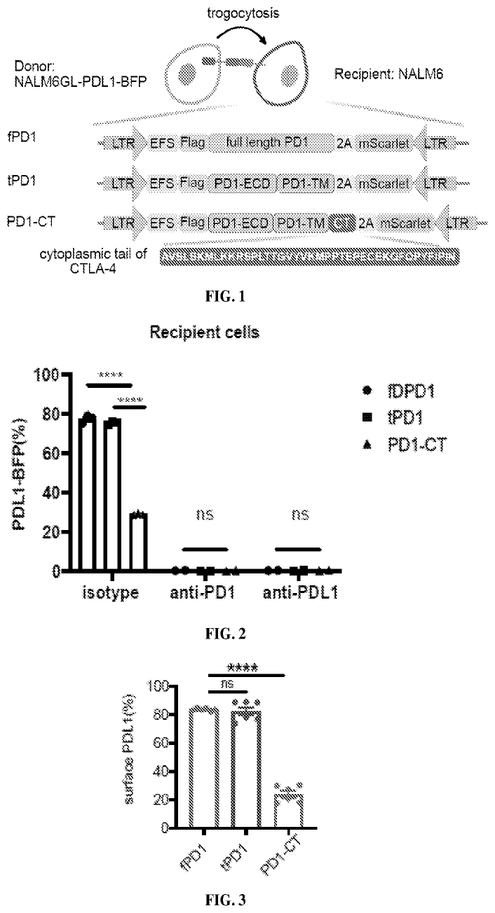

To determine if CTLA4 CT can be modularly fused to a different receptor and retain cycling function, CT was first engineered into a similar immune checkpoint factor, programmed cell death-1 (PD1) (FIG. 1), which belongs to the same co-inhibitory B7-CD28 family as CTLA-4, but lacks the dynamic intracellular regulation (Waldman, et al., Nat Rev Immunol 20, 651-668 (2020)). a trogocytosis experimental platform formed of donor and recipient cells, each derived from leukemia cell lines (NALM6) was established. Specifically, NALM6 donor cells were generated to express PD1 ligand 1(PD-L1) conjugated with BFP by a GGGS (SEQ ID NO:81) linker, which would interact with the recipient cells that express either a full length PD1 (fPD1), a truncated PD1 without the intracellular domain (tPD1), or chimeric PD1 with intracellular domain replaced by CT (PD1-CT). Compared with NALM6 recipient cells carrying fPD1or tPD1, PD1-CT NALM6 showed altered cellular localization pattern, with significant less PD1 at cell surface, while majority of the receptor was constantly cycling (FIGS. 6A-6D), similarly as what has been seen with highly endocytic CTLA-4 (Qureshi, et al., J Biol Chem 287, 9429-9440 (2012)) rather than native PD1. When cocultured, recipient cells with PD1-CT acquired significantly lower levels of PDL1-BFP than either fPD1 or tPD1 NALM6. It was demonstrated that PDL1-BFP transfer is specific to the PD1-PDL1 interactions, as the transfer of PDL1-BFP was completely abrogated by treatment with either anti-PD1 or anti-PD-L1 monoclonal antibodies (FIGS. 1 and 2).

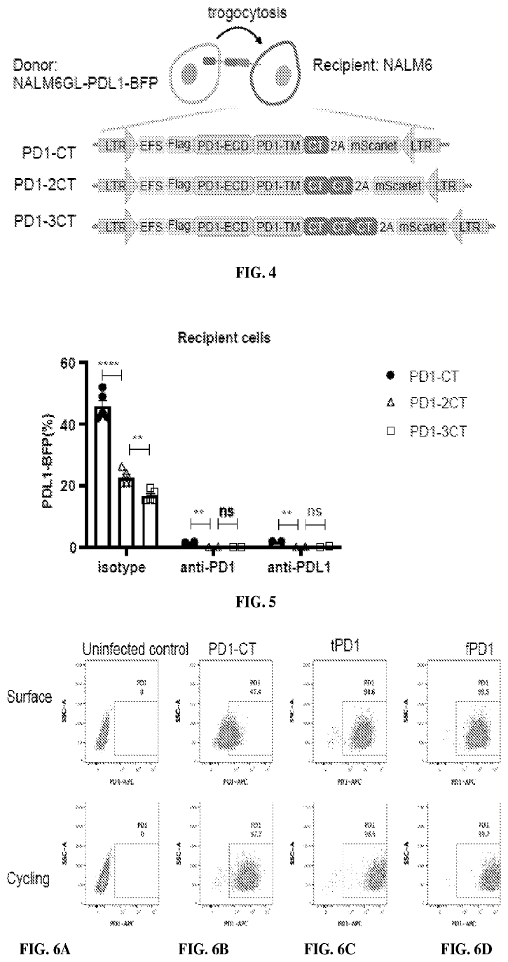

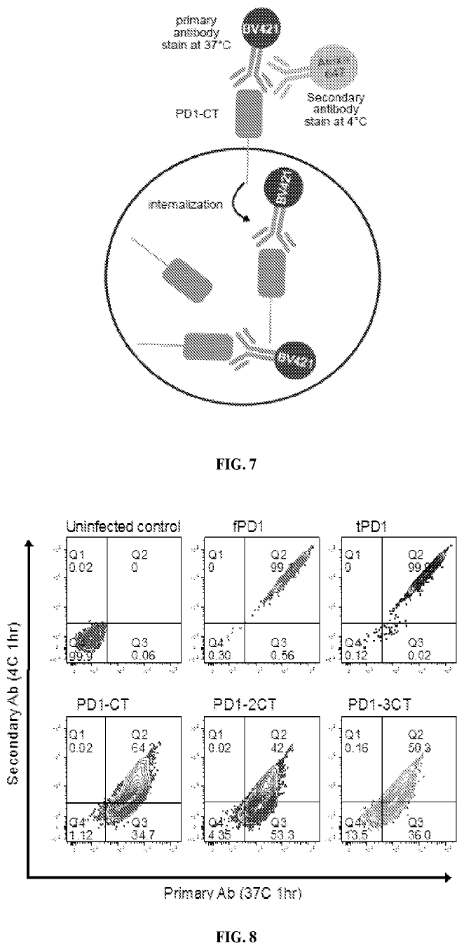

A similar amount of transferred PD-L1 between fPD1 and tPD1 NALM6 recipients (FIG. 2), indicating that the PD1 induced trogocytosis of PD-L1 is independent of its intracellular domain. Ligand transfer from giver to receiver cells was also quantified by cell surface staining of PD-L1 with anti-PD-L1 antibody. Less than 20% of trogocytosed recipient cells (BFP+ mScarlet+) carrying PD1-CT was stained by PD-L1 antibody. In contrast, cells carrying fPD1 or tPD1 were stained more than 80% (FIG. 3). Imaging of sorted BFP+ mScarlet+ recipient cells using confocal microscopy determined that the discrepancy between BFP signal and surface antibody staining might result from the higher intracellular distribution of PDL1-BFP trogocytosed by PD1-CT than by fPD1 or tPD1. Recipient cells with PD1-CT displayed distinct subcellular distribution of BFP, the majority of which was detected to be in cytoplasm rather than cell surface membrane, as seen in recipient cells carrying either fPD1 or tPD1; Confocal microscopy detection of BFP signal distribution on recipient cells that performed ligand uptake, with BFP+ recipient cells sorted by flow cytometer for confocal imaging indicated that CT alone is sufficient to redirect the intracellular localization of PD-1, a different surface protein, via control of trogocytosis. To investigate if additional CTs can further suppress receptor mediated trogocytosis, PD1-2CT and PD1-3CT constructs were generated by replacing the PD1 extracellular domain (ECD) with duplex or triplex CTs, respectively (FIG. 4). In comparison to PD1-CT, recipient cells expressing PD1-2CT and PD1-3CT showed further decrease in PDL1-BFP uptake (FIG. 5). Interestingly, a dose effect of CT was observed in controlling trogocytosis, with the more CT included, the less ligand transfer was induced by chimeric PD1 receptors (FIG. 5). To further characterize the cellular localization pattern of chimeric PD1 receptors, flow cytometry staining procedure that allows distinction between cycling PD1 and membrane PD1 (FIG. 7) was performed. The data showed that chimeric PD1 with CT (PD1-CT, PD1-2CT, PD1-3CT) had altered cellular localization patterns, with significantly decreased PD1 at the cell surface and the majority of the receptor constantly cycling, similar to the patterns of highly endocytic CTLA-4 and contrasting that of native PD1 (FIG. 8). Together, these results demonstrate the feasibility of using synthetic CTs to regulate chimeric receptors induced trogocytosis.

### Example 2: Engineered CARs with CT Fusion Confers CAR-T Cell Durability Under Repeated Stimulations

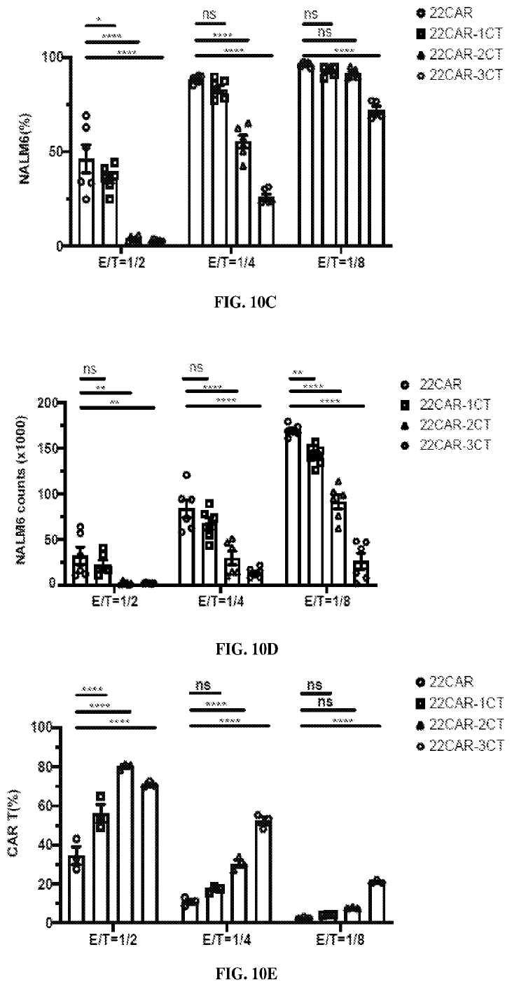

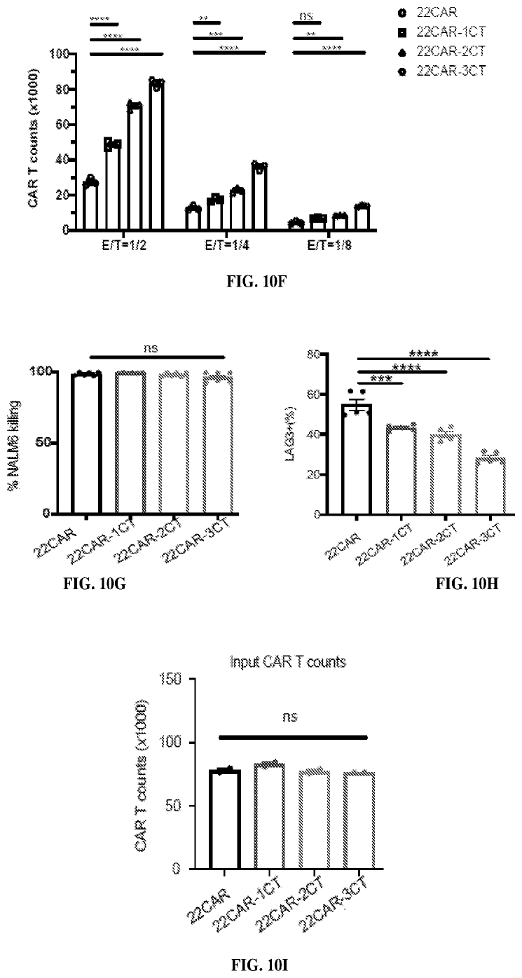

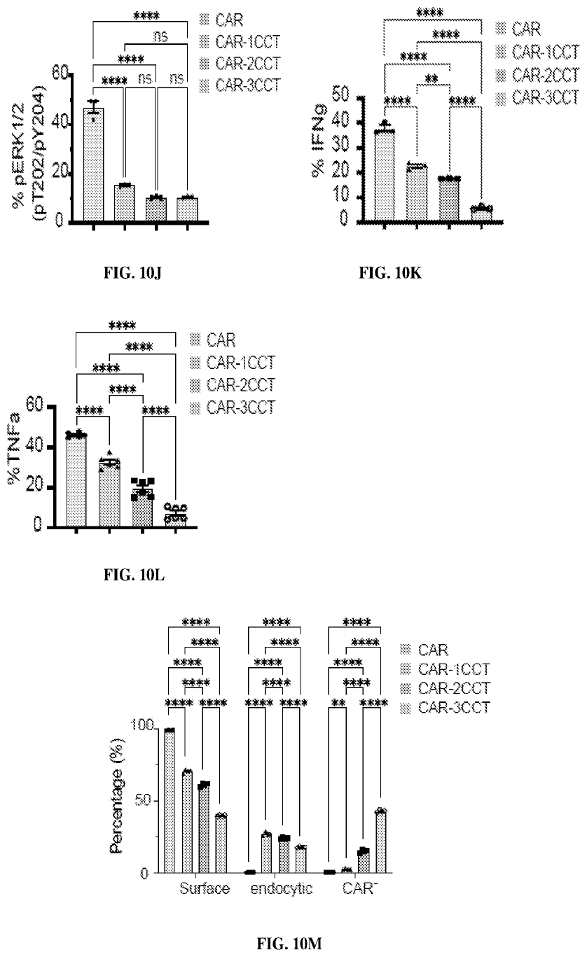

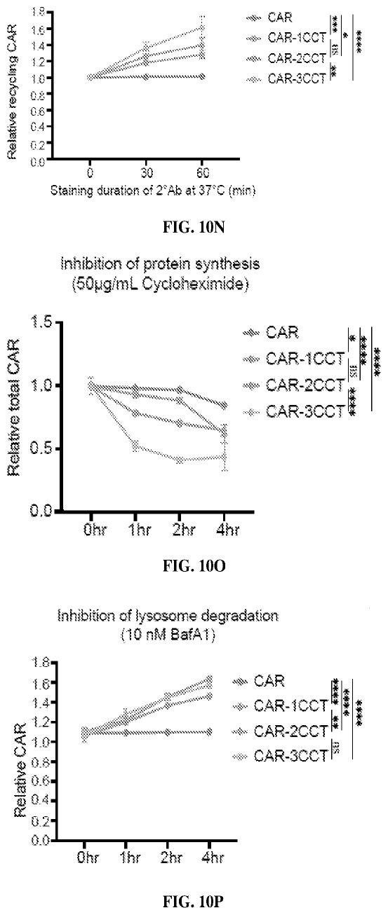

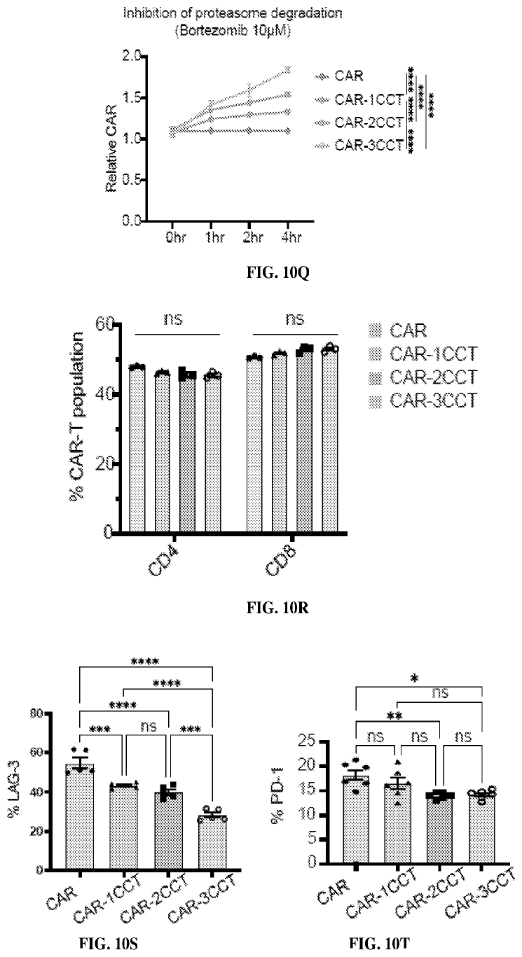

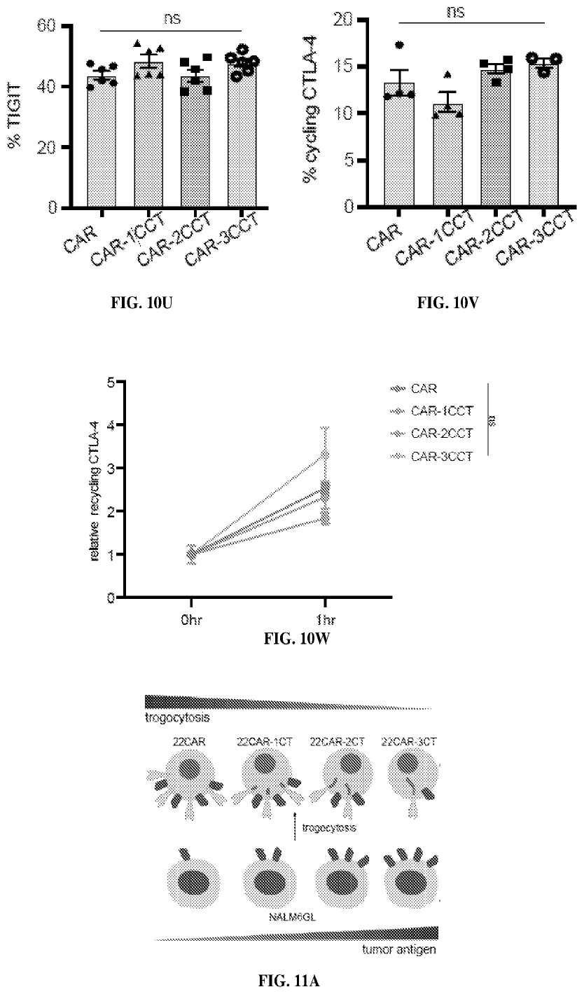

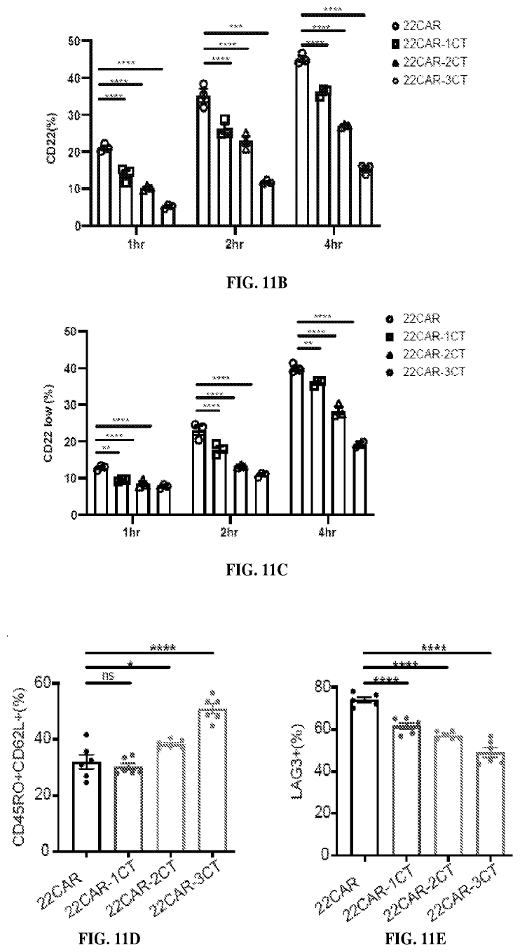

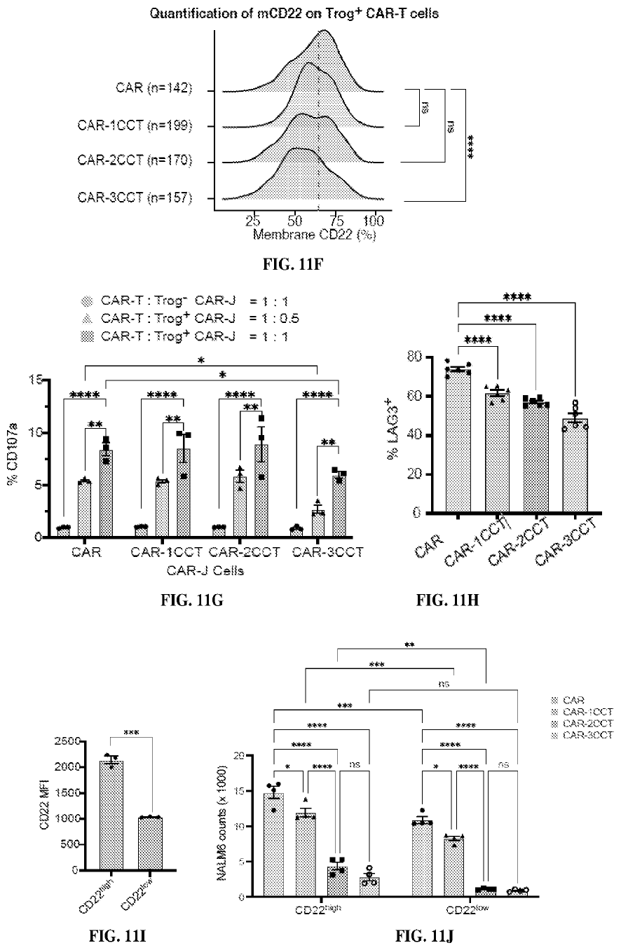

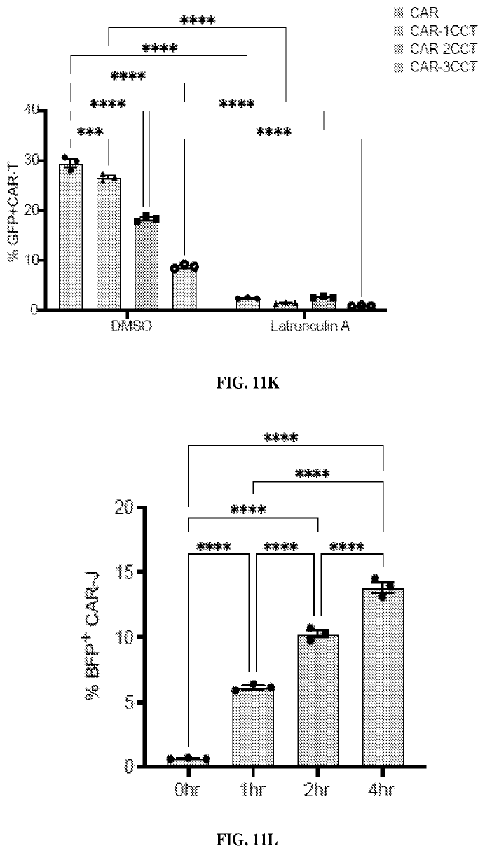

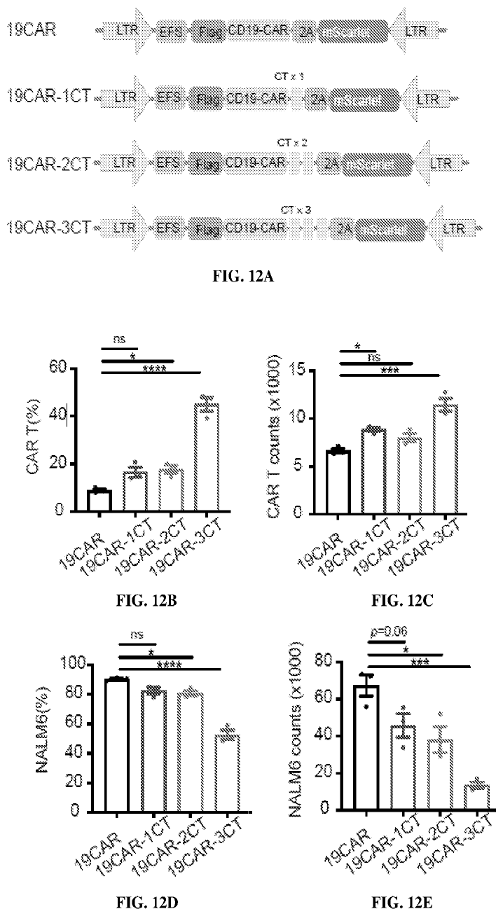

CAR-T cells have been shown to acquire surface molecules from tumor cells through trogocytosis, which causes tumor antigen loss and immune escape that compromise anti-tumor efficacy (Hamieh, et al., Nature 568, 112-116 (2019)). To test whether CT engineering can boost the function of CAR-T cells, CD22 CAR (22CAR) was designed, using the m971-BBz backbone, with monomeric (22CAR-1CT), duplex (22CAR-2CT), or triplex CT (22CAR-3CT) fused to its C terminus (FIG. 9A). In all these CAR constructs, a flag tag was also encoded at the N terminus for surface CAR detection, and an mScarlet reporter for transduction measurement (FIG. 9A-9C). In a dose (number of CTs) dependent manner, CT was observed to reduce surface CAR expression, as indicated by flag staining (FIG. 10A).

Human CD3 T cells were transduced with lentiviruses carrying the above constructs to generate CAR-T cells (FIG. 10A). Transduction with different constructs did not affect the CD4/CD8 T cell composition (FIG. 10R).

Effector function of engineered CAR-CT cells were assayed by coculturing them with NALM6 cells (B cell leukemia) expressing GFP-luciferase (NALM6GL) cells, followed bysimultaneous quantification of NALM6GL and CAR-T cells. All four groups of CAR-T cells lysed approximately 100% of NALM6GL when cocultured with one round of tumor cells for 24 hours (FIGS. 9A-9C, 10G). However, this was achieved with distinct consequences, significantly more CAR-T cells survived in groups of 22CAR-1CT, 22CAR-2CT and 22CAR-3CT (FIG. 10B) with lower level expression of T cell exhaustion markers LAG3, and PD-1 (FIG. 10H) than in the control 22CAR group. Other coinhibitory markers such as TIGIT, cycling CTLA-4, and recycling CTLA-4 were comparable between groups; TIM3 was undetectable in all groups (FIGS. 10S-10W).

To test that engineered CAR with CT(s) might support the persistent anti-tumor response of T cells under repeated antigen stimulation, a second round of NALM6GL was introduced 24 hours after the initial coculture (FIGS. 9B-9C). Compared with 22CAR, all groups of CAR-T cells with engineered CT(s) resulted in marked less live NALM6GL cells quantified as either percentage (FIG. 10C) or counts (FIG. 10I), indicating their superior capability of CAR-CT cells to kill NALM6GL. Similar to what had been observed in the one-round NALM6GL coculture, significantly more CAR-T cells in the groups of 22CAR-1CT, 22CAR-2CT and 22CAR-3CT survived the second round of NALM6GL stimulation (FIGS. 10E and 10F) with the same number of input CAR-T cells seeded at the beginning (FIG. 10I). Notably, after two rounds of cancer stimulation, 22CAR-2CT and 22CAR-3CT cells showed higher expression of central memory markers such as CD45RO and CD62L (FIG. 11D), while maintained lower LAG3 expression (FIG. 11E) than 22CAR-T cells.

To investigate the impact of tumor antigen density on this phenotype seen with CAR-CCT cells, NALM6-CD22-GFP cells were generated, in which CD22 was tagged with GFP by a GGGGS (SEQ ID NO:125) linker. CD22high and CD22low NALM6 cells were sorted out based on the CD22 expression level. In vitro coculture of CAR-CCT cells with either CD22high or CD22low NALM6 cells showed that CAR-CCT cells induced more efficient killing than the control CAR-T cells, with CAR-2CCT and CAR-3CCT showing the highest capability of killing both CD22high and CD22low NALM6 cells (FIG. 11H-11I). When repeatedly challenged with NALM6 cells, all four groups of CAR-T cells were more effective at killing cancer cells with lower CD22 expression (FIG. 11H-11I). These data indicate that, at least in the context of an in vitro co-culture system, a high cancer antigen load is unfavorable for CAR-T function under repeated challenge. Altogether, these results indicate that CAR-T cells with engineered CCT fusion have improved durability under repeated antigen stimulations, with enhanced cytolytic ability and a stronger central memory phenotype.

As the adopted approach involves engineering the distal cytoplasmic end of the CAR, it was sought to confirm its versatility by engineering CCT fusion to CAR targeting a different antigen. By replacing the m971 scFv with the FMC63 scFv, a series of CAR and CAR-CCT constructs were similarly engineered with a human CD19 targeting scFv (FMC63), namely CD19 CAR (19CAR), as well as monomeric (19CAR-1CT), duplex (19CAR-2CT), and triplex CTs (19CAR-3CT) (FIG. 12A). The experiments above were repeated with this series of CD19 CARs and similar results observed, where significantly more 19CAR-T cells survived the two rounds NALM6GL coculture (FIGS. 12B-12C), which progressively killed more NALM6GL cells with increasing number of CTs (FIGS. 12D-12E). Collectively, these results demonstrated that harnessing CT fusion to enhance CAR-T function is applicable to more than one type of CARs.

### Example 3: CAR-T with CCT Fusion Cells Show Decreased Activation and Production of Pro-Inflammatory Cytokines

The mechanisms underlying the better survival and increased killing of CAR-T cells with fusion CT(s) were explored. As seen from earlier experiments, CT(s) negatively regulate PD1 mediated PDL1 trogocytosis. To establish (i) whether the tumor antigen could be transferred onto CAR-T cells via trogocytosis, and thereby induce fratricide among CAR-T cells; and (ii) whether engineered CT(s) modulate these processes were assessed. By performing in vitro coculture of NALM6GL and CAR-T cells (FIG. 11A), substantial surface CD22 antigen was detected on CAR-T cells that normally do not express CD22, as early as 1 hour after the 22CAR-T cells were incubated with NALM6GL cells (FIG. 11B), indicating trogocytosis of CAR-T cells via 22CAR: CD22 interaction. Engineered CT(s) in CARs significantly reduced the trogocytosed surface CD22 level on CAR-T cells in a CT-dose-dependent fashion (FIGS. 11A-11B). Concordantly, the NALM6GL cancer cells retained progressively more surface CD22 antigen when co-cultured with 22CAR-T cells harboring increasing number of engineered CTs, as indicated by decreasing in CD22low population (FIG. 11C). These results showed that CT(s) efficiently reduced 22CAR mediated trogocytosis and CD22 loss in cancer cells.

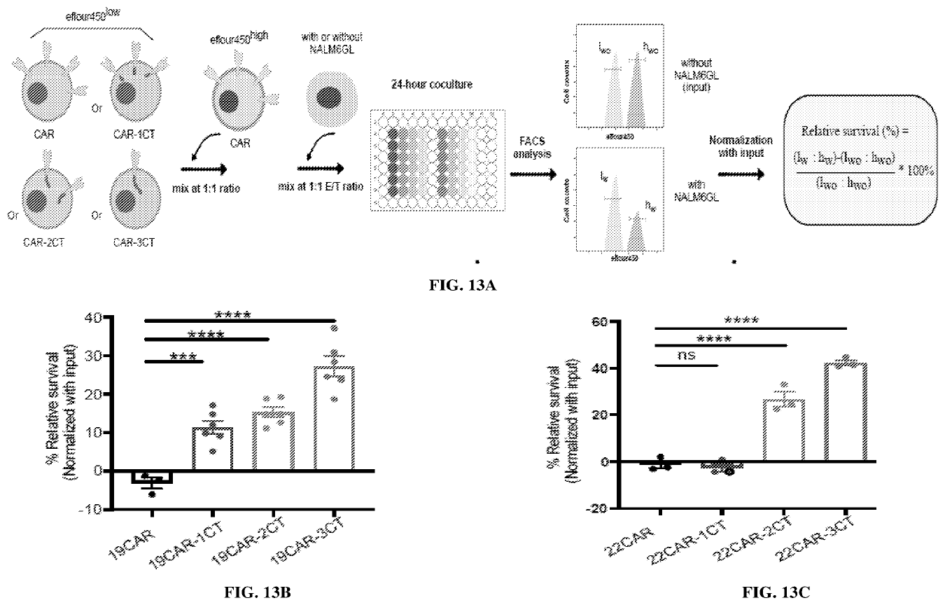

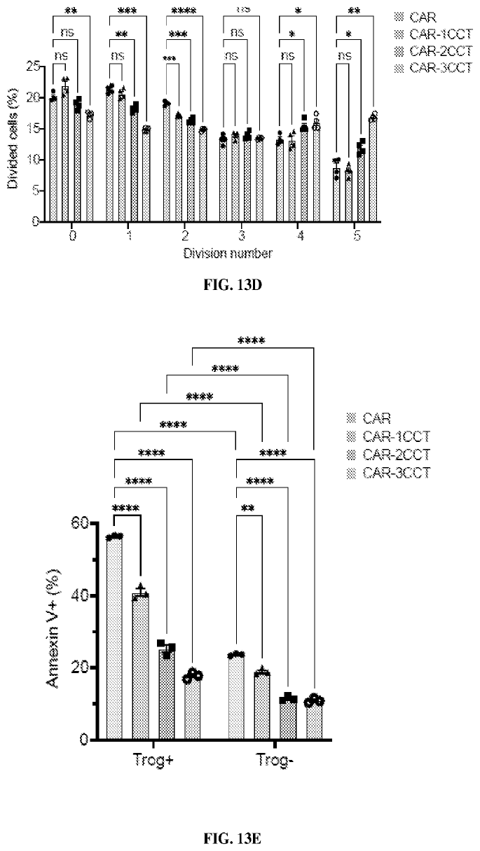

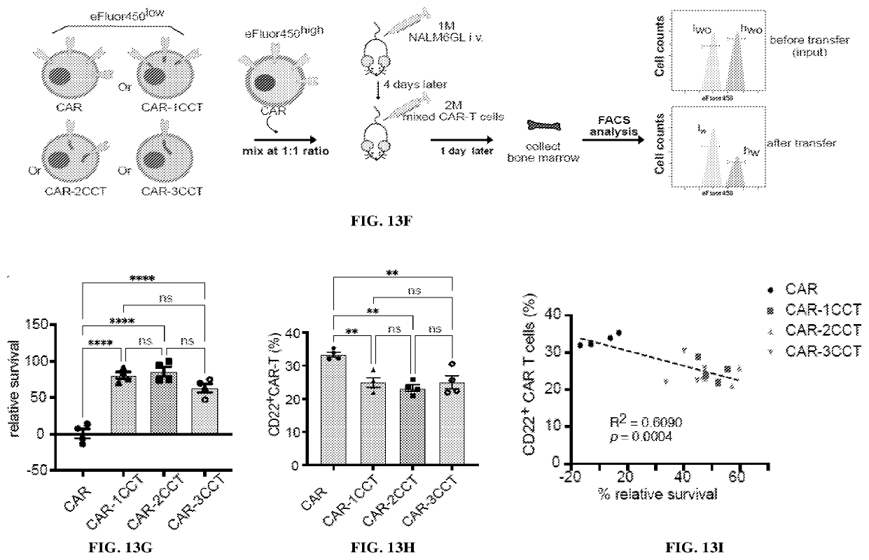

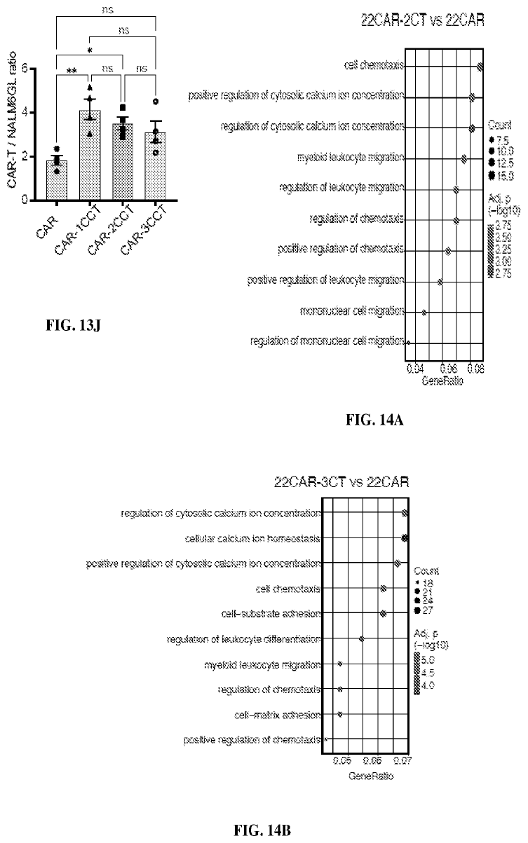

Fratricide among CAR-T cells by flow cytometry using a fratricide assay was then quantified (FIG. 13A). By doing this, it was found that both 19CAR-T (FIG. 13B) and 22CAR-T cells (FIG. 13C) exhibited significantly higher levels of survival in the groups with more CT copies fused to the CAR. This result indicates that CT fusion rescues CAR-T cells from high levels of fratricide once introduced with tumor cells.

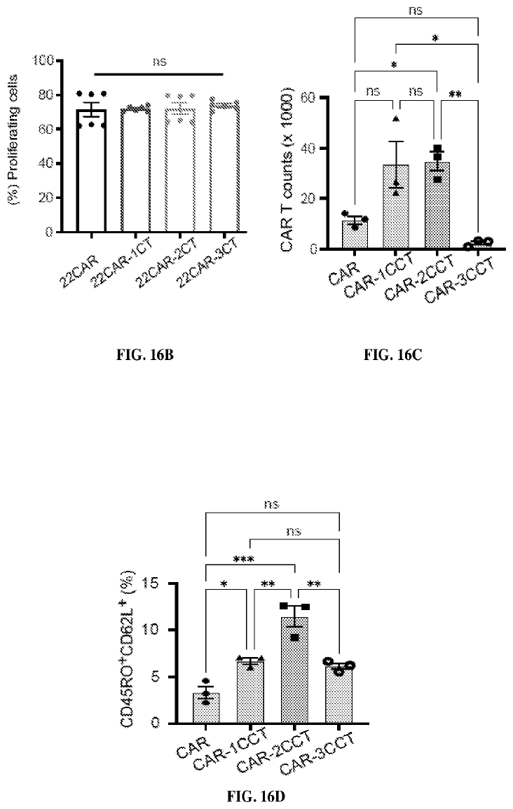

The secretion of cytokine and cytotoxic molecules, which are important for effector functions of CAR-T cells against tumor was also examined. The secreted proteins were quantified from the supernatants of cocultures with control 22CAR, 22CAR-1CT, 22CAR-2CT and 22CAR-3CT cells by LEGENDPlex bead-based immunoassays. The results showed that CT-engineered CAR-T cells, especially the 3CT group, had elevated levels of granzymes, perforin and granulysin (FIG. 16A), which are the main factors for targeted killing by CAR-T cells (Benmebarek et al., Int J Mol Sci 20, (2019)). On the other hand, CT-engineered CAR-T cells, in all three groups, had reduced levels of pro-inflammatory cytokines, such as IL-4, IL-6, IL-2, TNF-a, IL17a and IFNg (FIG. 16A), all of which are associated with cytokine release syndrome seen in clinical CAR-T therapy (Turtle et al., Sci Transl Med 8, 355ra116 (2016); Grupp et al., N Engl J Med 368, 1509-1518 (2013)). These results indicate that CT engineered CAR-T cells have a more potent effector function while minimizing the inflammatory response, frequently seen in CAR-T therapy.

To determine the possible contribution of cell proliferation to these results, CAR-T cells proliferation was quantified by flow cytometry with eflour450 labeling. Minimal and insignificant differences between all four groups of CAR-T cells were observed (FIG. 16B), ruling out the possibility that proliferation is a major contributing factor to the increased CAR-T cell survival with progressively more engineered CTs. These results together demonstrated that both CAR mediated trogocytosis and tumor antigen loss can be quantitatively suppressed with synthetic CTs, followed by decreased fratricide and elevated granule-exocytosis.

The data also indicate that engineered CAR-CT cells have improved killing capability with elevated degranulation, but decreased ERK phosphorylation, IFNg and TNFa production. Given the increased in vitro killing ability and elevated granzymes, CAR-CCT cells are more efficient in their ability to kill cancer cells. The accompanying decrease in ERK phosphorylation, IFNg, and TNFa production may imply a reduction in T cell activation and inflammatory responses, possibly due to alterations in CAR dynamics when CT is fused. The phenomenon that engineered T cells with enhanced cytotoxicity but low cytokine release have also been reported by others, potentially limiting the risk of cytokine release syndrome and cerebral edema/neurotoxicity, two of the major side effects associated with current CAR-T cell therapy.

### Example 4: CCT Fusion Enables Titration of CAR Expression Through Receptor Endocytosis, Recycling and Degradation at Steady State

CTLA-4 is constantly internalized through endocytosis, after which it is either recycled back to the cell surface or degraded. To gain insight into the improved killing ability and reduced activation of CAR-CCT cells, whether CARs with CCT fusion also possess similar molecular dynamics as native CTLA-4 was tested. It was first observed that CCT fusion reduced surface CAR expression in a dose-dependent manner (number of fused CCTs), as indicated by Flag staining after gating on mScarlet⁺ CAR-T cells (FIG. 10A). This reduction in surface CAR expression may explain the decreased activation level seen in CAR-CCT cells following antigen stimulation (FIGS. 10J-10K). To further characterize the cellular localization pattern of CAR-CCT molecules, a flow cytometry staining procedure that distinguishes between endocytic and surface CAR was performed. CARs with CCT fusion had altered cellular localization patterns and displayed endocytic features, with CAR-1CCT displayed the highest endocytosis level (FIG. 10M). Analogous to the results, it was again observed that with increasing CCT fusion, surface CAR levels decreased. CAR-3CCT cells had the highest number of mScarlet⁺ CAR⁻ populations (FIG. 10M), indicative of elevated degradation of CAR-3CCT molecules.

To characterize the recycling pattern of the CAR-CCT molecules, the staining protocol was further modified. Specifically, the levels of staining signal were compared when the secondary antibody was incubated at 4° C. for 15 minutes or at 37° C. for either 30 or 60 minutes. Compared to the 4° C. incubation, an increase of staining at 37° C. for either 30 or 60 minutes is indicative of active recycling CAR molecules. It was found that in contrast to the control CAR, all CAR-CCT molecules showed recycling features, with CAR-3CCT showing the highest recycling rate, and CAR-1CCT and CAR-2CCT demonstrating similar recycling rates (FIG. 10N).

The stability of the CAR-CCT molecules at steady state was quantified. By inhibiting de novo protein translation with cycloheximide, it was found that CAR-CCT molecules had significantly decreased stability, with CAR-3CCT molecules demonstrating the highest degradation rate (FIG. 10O). Next, the pathway through which CAR-CCT molecules were being degraded was investigated. As native CTLA-4 is subjected to both lysosomal and proteasome degradation, their contribution to CAR-CCT stability was investigated by introducing Bafilomycin A1(BafA1, lysosomal inhibitor) or bortezomib (proteasomal inhibitor). BafA1 or bortezomib treatment significantly increased relative CAR expression in CAR-CCT groups over time, while no clear change was observed in the control CAR group, indicating that both the lysosome and proteasome are involved in CAR-CCT degradation (FIGS. 10P-10Q). Interestingly, a CCT fusion dose-dependent effect was observed when the cells were treated with bortezomib, indicating that with increasing CCT fusions, proteasomal degradation of the CAR-CCT is also increased (FIG. 10Q). In contrast, there was no difference in CAR expression between CAR-3CCT and CAR-2CCT when lysosomal degradation was inhibited by BafA1 (FIG. 10P), though 2CCT and 3CCT were nevertheless subject to more lysosomal degradation than 1CCT or the baseline CAR. These results indicate that CCT fusions promote lysosomal degradation, but only up to certain point (2CCT); on the other hand, increasing CCT fusions progressively promote proteasomal degradation at least up to 3CCTs. Collectively, these data indicate that the surface expression of CAR-CCT molecules is regulated by their constant endocytosis, recycling and degradation under steady state, contrasting with that of the control.

### Example 5: CCT Fusion Reduces CAR-Mediated Trogocytosis

CAR-T cells have been shown to acquire surface molecules from tumor cells through trogocytosis, leading to antigen loss that compromises anti-tumor efficacy. In line with these prior observations, time-lapse live cell imaging was performed and it was found that CAR-T cells actively acquired CD22 antigen from NALM6GL cells. By including a dye that only penetrates dead cells, active fratricide was recorded among CAR-T cells, as indicated by influx of the dye after an active engagement between CD22⁺ CAR-T cells. Moreover, it was found that latrunculin A, an F-actin inhibitor, inhibit the transfer of CD22 antigen from NALM6 cells to T cells, a key hallmark of CAR-mediated trogocytosis (FIG. 11K), which further support the observations.

It has been reported that trogocytosis is impacted by CAR affinity, and whether the altered molecular dynamics of CAR-CCT might also impact its propensity for mediating trogocytosis was tested. From in vitro cocultures of NALM6GL and CAR-T cells, substantial surface CD22 antigen was detected on CAR-T cells that normally do not express CD22, as early as 1 hour after incubation (FIG. 11B), consistent with rapid trogocytosis by CAR-T cells. Engineered CCT(s) in CARs significantly reduced trogocytosis-mediated acquisition of surface CD22 in a CCT-dose-dependent fashion (FIGS. 11B, 10B). Concordantly, the NALM6GL cancer cells retained progressively more surface CD22 antigen when co-cultured with CAR-T cells harboring increasing numbers of engineered CCTs (FIG. 11C). These results showed that CCT fusion efficiently reduces CAR-mediated trogocytosis and CD22 loss in cancer cells.

As CTLA-4 has been shown to internalize ligands acquired by trans-endocytosis the localization of the transferred CD22 antigen was quantified by sorting out CD22⁺ CAR-T (Trog⁺) cells for confocal imaging. In all groups, the majority of the transferred CD22 antigen localized to the cell membrane (FIG. 11F). Of note, only very high levels of CCT fusion (3CCT) seemed to decrease membrane colocalization, given that a significant leftward shift of the membrane CD22(%) distribution was only observed for CAR-3CCT (FIG. 11F). Thus, the presence of 1CCT or 2CCT fusion does not significantly change the surface localization of CD22 molecules obtained through CAR-mediated trogocytosis, whereas CAR-3CCT fusion leads to increased internalization.

To determine if the CD22 antigen acquired by trogocytosis is still functional in stimulating CAR-T cells, CAR-Jurkat (CAR-J) cells, which undergo trogocytosis of target CD22 were generated (FIG. 11C) without killing the target cells. After transducing, Jurkat cells with different CAR-CCT constructs were incubated with NALM6-CD22-GFP cells to allow for CD22-GFP transfer onto Jurkat cells. Both CD22-GFP⁺ (Trog⁺) and CD22-GFP⁻ (Trog⁻) CAR-J cells were sorted out and used as target cells to stimulate fresh CAR-T cells (effector cells) that had been labeled with eFluor450. In all groups, Trog⁺ CAR-J cells could induce the cytotoxic degranulation of CAR-T cells, as indicated by the membrane expression of CD107a. This was achieved in a dose-dependent manner, as higher degranulation occurred when more target Trog⁺ CAR-J cells were added to the co-culture (FIG. 11G).

Given that significantly lower percentages of Trog⁺ were observed in the CAR-CCT-T cells after their encounter with cancer cells (FIG. 11B), lower degranulation, and thus lower fratricide among them could be inferred. In comparison, Trog⁻ CAR-J cells did not meaningfully induce degranulation, as expected (FIG. 11G). These data indicate that CD22 transferred through trogocytosis is functional in stimulating CAR-T cell degranulation. While the stimulation of CAR-T degranulation was similar among Trog⁺ control CAR-J cells, CAR-1CCT-J and CAR-2CCT-J cells, Trog⁺ CAR-J cells with 3CCT fusion induced significantly lower degranulation (FIG. 11G). This may result from the enhanced internalization of transferred CD22 by CAR-3CCT, as seen in FIG. 11F. Together, these results indicate that CCT fusion quantitively reduces CAR-mediated trogocytosis, loss of cancer antigen.

### Example 6: CCT Fusion Enhances the Survival and Proliferation of CAR-T Cells

Given that CAR-CCT cells were more abundant after repeated antigen stimulation and also exhibited reduced trogocytosis, whether CCT fusion impacted CAR-T cell survival and proliferation was tested. To test this, CAR-CCT cells were mixed with control CAR-T cells labeled with eFluor450 dye, followed by NALM6GL stimulation. This ensured that both the CAR-CCT and control CAR-T cells were subjected to the same stimulation (FIG. 13A). Quantifying the relative abundance of eFluor450^{high} and eFuor450^{low} cells, it was found that CAR-T cells with CCT fusion exhibited improved survival than the control (FIGS. 13B-13C). To assess the potential role of cell proliferation in these findings, flow cytometry and eFluor450 labeling were used to quantify CAR-T cell proliferation following stimulation with NALM6GL cells. CAR-2CCT and CAR-3CCT cells exhibited greater proliferation potential, as more cells underwent 4 or 5 rounds of division in these two groups (FIG. 13D). In an analogous co-culture experiment, it was further observed that Trog⁺ CAR-T cells were significantly more apoptotic than the Trog⁻ CAR-T cells, and CAR-T cells with increasing CCT fusion had reduced apoptosis levels (FIG. 13D). These results indicate that the high levels of trogocytosis induced by the control CAR are unfavorable for T cell survival, and that CAR-CCT fusion inhibits this process to improve CAR-T survival following antigen exposure.

Whether the enhanced survival of CAR-CCT cells was also evident in the in vivo setting was next investigated. In mice intravenously injected with NALM6GL cells, adoptively transferred CAR-T cells from bone marrow, where most cancer cells reside, were harvested for analysis. It was found that CAR-T cells engineered with CCT showed significantly higher relative survival (FIG. 13F-13G) and lower trogocytosis levels (FIG. 13H) compared to those without CCT. Interestingly, a strong correlation between the relative survival and trogocytosis of the CAR-T cells was observed (FIG. 13I), with higher survival corresponding to lower trogocytosis. This was consistent with the results showing higher CAR-T: NALM6GL ratios in CAR-T cells engineered with 1CCT or 2CCTs compared to the control CAR-T cells (FIG. 13J). These findings collectively demonstrate that CCT fusion effectively improves the survival and proliferation of CAR-T, and impairs the high level of apoptosis associated with trogocytosis.

### Example 7: CAR-T Cells with CCT Fusion Show Reduced Tonic Signaling and Increased Responsiveness to Repeated Stimulations at Transcriptome Level

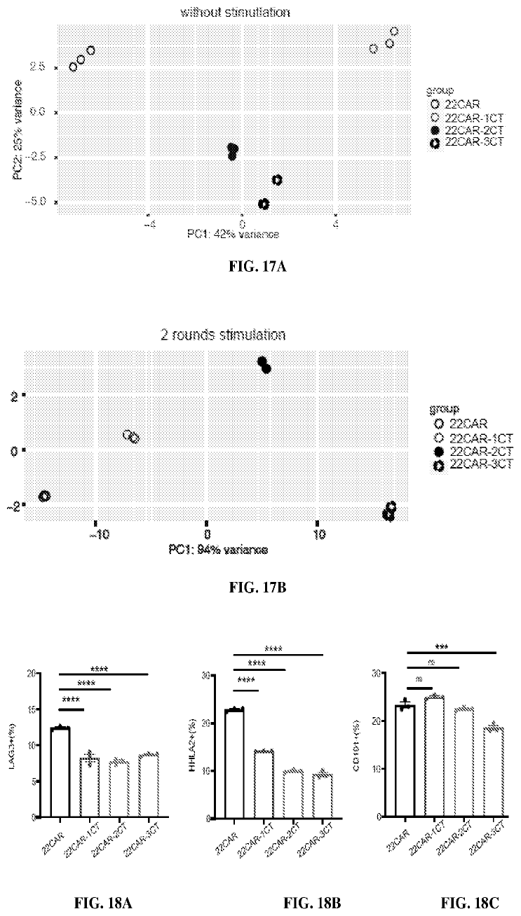

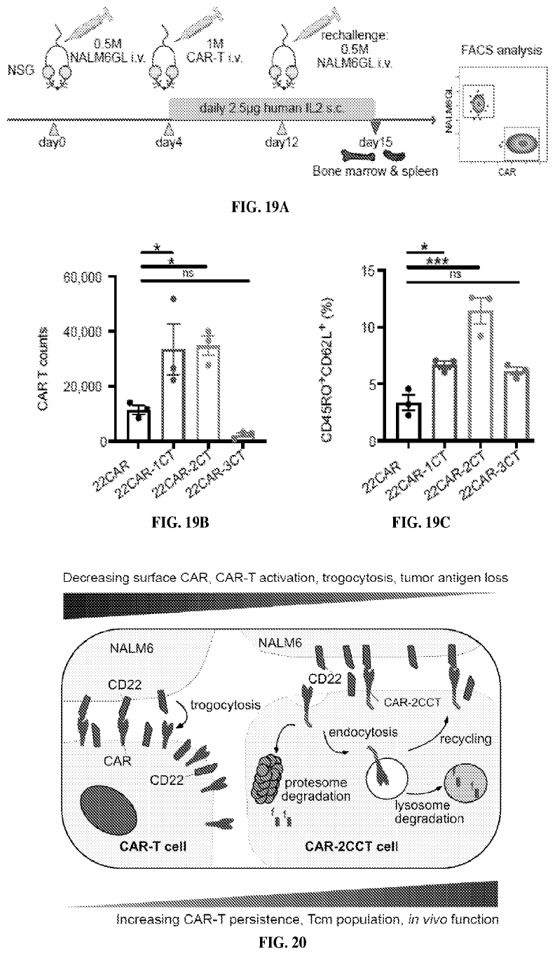

CAR-T Cells were Characterized Using
transcriptome profiling. To unbiasedly profile the transcriptomic differences among CAR-T cells with different numbers of CTs, mRNA-seq was performed from all four groups of CAR-T cells either without stimulation (baseline) or with 2 rounds of NALM6GL stimulation. For CAR-T cells that were cocultured with 2 rounds of NALM6GL cells, the pure CAR-T populations (CAR+; GFP−) were sorted before subjecting them to RNA sequencing. The RNA-seq data showed overall high quality, where unbiased principal component analysis (PCA) showed distinct group separation between control 22CAR, 22CAR-1CT, 22CAR-2CT and 22CAR-3CT groups (FIG. 17A-17B). At baseline without NALM6GL stimulation, 22CAR-1CT, 22CAR-2CT and 22CAR-3CT cells displayed reduced tonic signaling as indicated by marked reduction in inhibitory receptors, including LAG3, CD101, HHLA2, CD160, KLRB1 and CD244. High expression of these genes is consistent with antigen independent activation of CAR, which is the hallmark of tonic signaling and can ultimately lead to exhaustion (Gomes-Silva, et al., Cell Rep 21, 17-26 (2017); Long, et al., Nat Med 21, 581-590 (2015)). This finding was further validated at protein level by flow cytometry analysis of LAG3, HHLA2 and CD101 (FIG. 18A-18C).

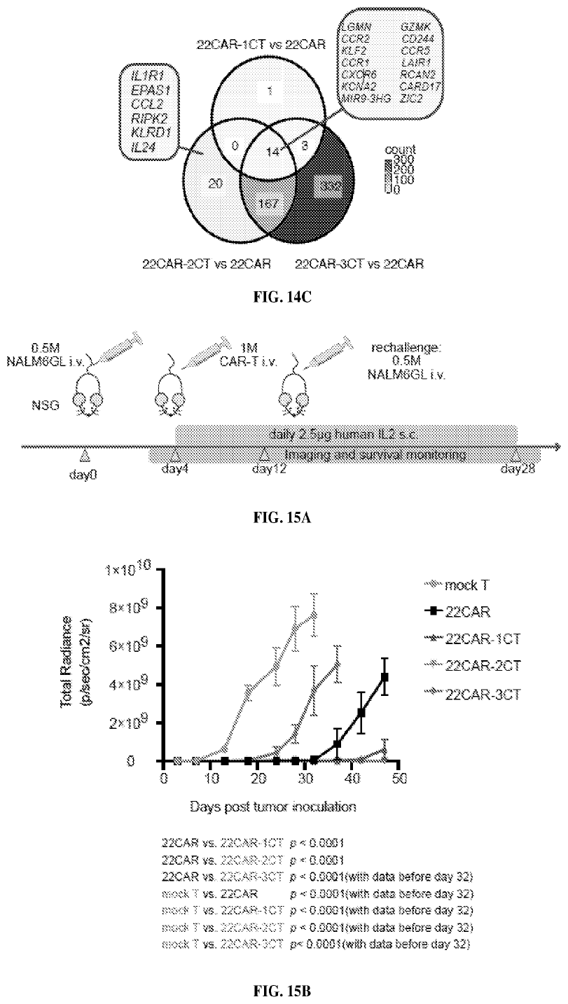

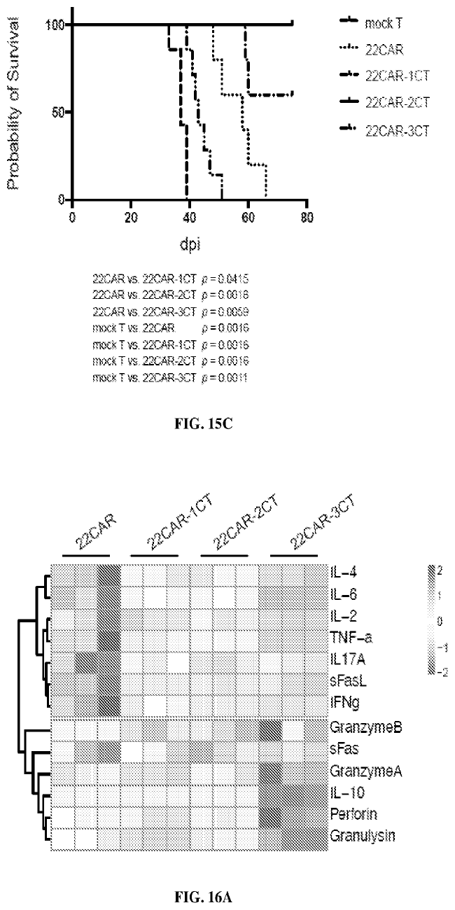

From RNA-seq differential expression (DE) analysis of CAR-T cells that underwent repeated cancer stimulations, a single synthetic CT can result in a global transcriptome alteration (FIG. 12A). Compared 22CAR-1CT with the control 22CAR-T cells, a total of 317 genes were found to be significantly upregulated and 275 genes were significantly downregulated (DE genes, at adjusted q<0.01 level) (FIG. 12A). 22CAR-2CT cells showed 2107 upregulated and 1592 downregulated genes, and 22CAR-3CT cells showed 3356 upregulated and 2811 downregulated genes (FIG. 15A). The DE genes were further filtered by fold changes compared to the control (log 2Fcl>1). Pathway analysis of these gene sets (DE-FC genes, significant and with at least 2-fold change) revealed a number of enriched differentially altered pathways (FIGS. 14A-14B). For example, 22CAR-2CT cells upregulated genes that are enriched in cell chemotaxis, immune cell/leukocyte migration and regulation of cytosolic calcium ion concentration, which are indicative of enhanced responsiveness of CAR-T cells to repeated tumor antigen stimulations (FIGS. 14A-14B). Among the commonly upregulated genes, GZMK, CD244, CCR2, CCR5, CXCR6 and KLF2 are related to effector T functions, which were significantly upregulated in all three groups of 22CAR T cells with CTs (FIG. 14C). Notably, 20 DE-FC upregulated genes unique to the 22CAR-2CT T cells were observed, including IL1R1, EPAS1, CCL2, RIPK2, KLRD1 and IL24 (FIG. 14C). Among these, IL1R1(Lee, et al., J Exp Med 216, 2619-2634 (2019)) and EPAS1 (Velica et al., Cancer Immunol Res 9, 401-414 (2021).)(encodes hypoxia-inducible factor 2a, HIF2a) were reported to directly enhance the effector function of adoptively transferred T cells against tumors. Together, these results demonstrate that CT used for CAR-T cell engineering can reduce tonic signaling of CAR at baseline while preserving high responsiveness of CAR-T cells to repeated antigen stimulations.

### Example 8: Engineered CAR-T Cells with Monomeric or Duplex Fusion CTs Exhibit Substantially Enhanced In Vivo Therapeutic Efficacy in a Mouse Model of Relapsed Leukemia

To assess the in vivo performance of the synthetic CT fusion CAR-T cells, a relapsed leukemia mouse model was established (FIG. 15A). In this model, NSG mice first underwent disease induction with 0.5 million of luciferase-expressing NALM6GL cells intravenously (i.v.) injected on day 0, and treated with 1 million CAR-T cells (i.v.) on day 4. Using bioluminescence imaging of disease progression by IVIS, all mice that underwent treatment of all four CAR-T groups (22CAR-1CT, 22CAR-2CT, 22CAR-3CT and control 22CAR) had no detectable disease at day 7. Disease relapse was modeled by re-challenging with 0.5 million NALM6GL cells (i.v.) on day 12, when no luciferase signal was detected in any mice across all groups treated with CAR-T cells. To support in vivo CAR-T cell survival and better model the clinical response in NSG mice (Jespersen, et al., Nat Commun 8, 707 (2017)), all mice were subcutaneously (s.c.) administered with 2.5 μg human recombinant IL2 everyday starting on day 4 for 24 days.

By performing IVIS imaging, relapse was detected in mice with the control 22CAR group as early as day 37, when no clear luciferase signal was detected in mice from 22CAR-1CT and 22CAR-2CT groups (FIG. 15B). 22CAR-1CT cells efficiently controlled leukemia development in NSG mice, leading to significantly reduced tumor burden and significantly better survival than the control 22CAR-T cells (compared to control 22CAR, p<0.0001 for tumor burden, p=0.04 for survival) (FIGS. 15C-15D). Surprisingly, mice treated with 22CAR-3CT cells had earlier relapses than those with 22CAR-T cells, indicating that triplex CT did not improve in vivo CAR-T efficacy over the control CAR-T cells. 22CAR-2CT cells had remarkably enhanced efficacy, in which all mice are in full remission against two cancer challenges, with 100% survival and nearly no detectable tumor burden (compared to the control 22CAR, p<0.0001 for tumor burden, p=0.0018 for survival) (FIGS. 15B-15C). These data indicate that engineering CAR-T with single CT significantly enhanced in vivo therapeutic efficacy against relapsed leukemia, while CAR-T with duplex CTs had superior efficacy, yet CAR-T with triplex CTs did not.

To further understand the reasons why CAR-1CT and CAR-2CT cells have superior in vivo efficacy when compared to CAR-3CT cells, immunological characterization of all these CT engineered CAR-T cells was performed in an in vivo model similar to that used in the efficacy measurement (FIG. 19A). As compared to control CAR-T cells, CAR-1CT and CAR-2CT cells were found, but not CAR-3CT cells, displayed significantly higher abundance in the spleen (FIG. 19B). Further characterization of T cell memory phenotype reveals that 22CAR-1CT and 22CAR-2CT cells had a notably higher fraction of central memory T cells populations (Tcm, CD45RO+;CD62L+) as compared to the control 22CAR-T cells (FIG. 19C); whereas CAR-3CT cells did not have significant increase over the control (FIG. 19C). These data showed that engineering with 1CT and 2CT, but not 3CT, increased CAR-T cells' in vivo persistence and central memory formation, in accordance with their superior anti-leukemia efficacy in vivo.

To systematically investigate the effect of CCT engineering on CAR-T cell phenotypes in vivo, single-cell RNA sequencing (scRNA-seq) was used to characterize the full spectrum of engineered CAR-T cells and their transcriptomic profiles. CAR-T cells were isolated from both spleen and bone marrow of the mice from the same leukemia mouse model (FIG. 19A). To minimize sampling bias, samples were pooled from 3 individual mice within the same group for all four groups (CAR, CAR-1CCT, CAR-2CCT and CAR-3CCT). CAR-T cells were isolated from these pooled samples via fluorescence-activated cell sorting (FACS) based on the expression of mScarlet. superior anti-tumor efficacy, the transcriptomes of single CAR-T cells were mapped via Uniform Manifold Approximation and Projection (UMAP), and identified in 12 distinct clusters. Those clusters were annotated based on their expression profile of known immune-cell markers. When annotating based on CD4 and CD8A expression, it was observed that CD8 CAR-T and CD4 CAR-T cells included 8 and 4 clusters, respectively.

Heterogeneity within these populations was further resolved based on the expression of naïve/memory markers (CD62L, TCF7, LEF1, CD27 and CD28), effector/cytotoxicity associated markers (GZMK, PRF1), activation/exhaustion markers (CD226, TOX, TIGIT and LAG3), tissue residency marker (ZNF683) and proliferation marker (MK167). In terms of tissue distribution, samples isolated from bone marrow included a higher level of heterogeneity than those from spleen, as more diverse T cell states were identified in CAR-T populations from bone marrow than spleen.

scRNA-seq of the CCT engineered CAR-T cells (CAR-1CCT, CAR-2CCT and CAR-3CCT) revealed significant changes in cell subset composition compared to the CAR control group in the bone marrow, but not spleen. An increase of CD8 Tcm1, CD8 Tcm2, proliferating (prolif.) CD8 Tem and CD8 tissue resident T (CD8 Trm) cells was observed a in CAR-1CCT and CAR-2CCT particularly, relative to the control CAR group. Consistent with the flow cytometry results, major differences were observed in the abundance of Tcm cells between CCT engineered CAR-T cells and control CAR-T cells. CAR-2CCT cells from bone marrow had the highest proportion of CD8 Tcm cells among all groups. Gene DEGs between CAR-2CCT and CAR-3CCT, which had the most distinct in vivo efficacy were further analyzed. In most T cell clusters, the number of DEGs are small. In the bone marrow CD4 Teff cluster, effector makers like KLRB1, KLRK1, KLRG1, NKG7, GZMK and CTSW were significantly upregulated in the CAR-2CCT compared with CAR-3CCT group. This indicates that CD4 Teff cells from the CAR-2CCT group have a more activated and cytotoxic phenotype compared to those from the CAR-3CCT group. Taken together, these data indicate that the improved in vivo efficacy achieved by CAR-1CCT and CAR-2CCT cells is linked to their heightened persistence and ability to differentiate into Tcm cells when faced with repeated challenges.

### SUMMARY

Cell-based therapeutics have demonstrated great promise with six CAR-T cell therapies currently approved by the FDA and over one thousand active clinical trials involving cell therapies (clinicaltrials.gov). Despite these advances, durable remissions are only observed in a limited number of patients. Improving the clinical efficacy of CAR-T cell therapy thus necessitates the development of new strategies to prevent relapse. By harnessing the unique dynamic features of the CTLA-4 cytoplasmic tail (CCT), CAR-T cells in which the CAR is fused to varying numbers of CCTs were developed. Mechanistically, CCT fusion altered the dynamics of CAR molecules by accelerating CAR endocytosis, degradation and recycling, processes that were observed were infrequently occurring with native CARs. This in turn led to reduced CAR-T tonic signaling and dampened T cell activation and inflammatory cytokine production. Additionally, CCT fusion further limited trogocytosis and cancer antigen loss, ameliorating the potential for subsequent CAR-T fratricide. In turn, CAR-1CCT and CAR-2CCT cells showed improved survival and persistence in the context of a leukemia mouse model, with enhanced anti-cancer functionality upon repeated cancer stimulation, increased in vivo persistence, and enrichment for Tcm differentiation (FIG. 20).

Trogocytosis is well-documented process that mediates surface molecule transfer from target cells to many immune populations such as T, B and NK cells. Recently, CAR mediated trogocytosis was reported to impair the anti-tumor efficacy of both CAR-NK and CAR-T cell-based therapies. However, to date there are no established strategies that can be generally applied to reduce CAR-mediated trogocytosis. CCT fusion approach introduces a new molecular feature to the CAR, enabling dynamic control of surface CAR expression at the protein level and effectively reducing CAR-mediated trogocytosis. Importantly, by adjusting the number of CCT fusions, quantitative regulation of CAR-mediated trogocytosis was demonstrated (FIGS. 11B-11C). This study therefore builds on previous work showing that the frequency of trogocytosis is impacted by the density of target antigen and the affinity of CAR. This study is the first to show that the molecular dynamics of CARs and CAR-mediated trogocytosis can be quantitatively regulated by adjusting the number of CCT fusions. This approach involves engineering the distal cytoplasmic end of the CAR, which might be further tested and applied to receptors with different antigen specificities. The properties of CCT fusion are also applicable to CD19-specific CARs, indicating that it is not limited to one type of CAR (CD22 CAR).

Besides the regulation of trogocytosis, CCT fusion effectively improved CAR-T survival in vitro and in vivo (as shown in FIGS. 10D-10F, FIGS. 13B-13C), while also leading to reduced apoptosis (FIG. 13E). While the reduction in trogocytosis and potentially fratricide among CAR-T cells with CCT likely contributes to this phenomenon, other possible explanations such as increased proliferation (FIG. 13D) and reduced cell activation-induced death are also plausible explanations.

Interaction strength between CAR and its cognate antigen is a fundamental determinant of CAR-T function. In line with this, the data show that regulating CAR signaling strength with CCT fusion prevents CAR-T cells from excessive stimulation and enables durable response against tumor cells. CAR signaling needs to be tightly regulated to achieve the fine-tuned balance of sufficient signaling for effective tumor antigen detection and CAR-T action, while also circumventing the detrimental effects seen in CARs with an excessively high affinity or expression. This resonates in the results with CAR-3CCT cells, as these cells exhibited the best killing capability under repeated stimulation in vitro yet performed worse than the control CAR-T cells in vivo. One possible explanation is that the surface availability of CAR needed for optimal signaling strength is different in vitro vs in vivo. In vitro co-culture assays have artificially high density and close proximity of CAR-T cells to tumor cells; in this context, reduced CAR surface expression (as seen in CAR-3CCT) is potentially advantageous by sufficiently mediating tumor cell killing while mitigating drawbacks such as exhaustion, activation-induced cell death, and fratricide. However, CAR-CCT cells are more diluted in the in vivo setting, and perhaps higher CAR surface expression and signaling strength is crucial to overcome the lower density of CAR-CCT cells in vivo. CAR-2CCT cells may land in a local optimum for in vivo efficacy with balanced cellular features. More generally, this study highlights the importance of in vivo studies to titrate the signaling strength induced by CARs for sustained anti-tumor responses, as in vitro co-culture assays often cannot recapitulate such complexities.

Other studies have proposed approaches to modulate CAR signaling strength, such as transient cessation of CAR signaling with a multi-kinase inhibitor, lower affinity CARs, and regulated promoters. Distinct from these studies, CCT fusion, with the merit of simplicity, enables tunable regulation of CAR surface availability without additional chemicals, without the need to engineer new scFVs of varying affinity. CCT fusion effectively regulates CAR availability at the protein level, in certain cases advantageous over transcriptome level regulation (e.g. inducible promoters) because it provides direct control over the amount of CAR protein present, as the relation between transcriptional levels and downstream protein levels is often complex. CCT fusion also opens up a potential avenue to generate CARs with self-regulation according to antigen stimulation level. For instance, fused CCT(s) could be cleaved in a regulatable manner by introducing a protease module that is responsive to antigen stimulation, thus enabling control over the timing and duration of CAR expression. This self-regulation would allow for flexible and dynamic self-titration of CAR signaling, which will be valuable in optimizing efficacy while also limiting toxicity.

In conclusion, the study demonstrates that fine-tuning CAR dynamics with CCT fusion can improve the anti-tumor efficacy of CAR-T-cell therapy, illuminating an orthogonal strategy for the development of more effective CAR-T-cell therapies. Future studies can explore the generalizability of this tail fusion strategy, including different protein tails, different cancer types, different CARs and different types of therapeutic immune cells.Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different gene targets does not indicate that the listed gene targets are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize or be able to ascertain using no more than routine experimentation, many equivalents to the specific embodiments of the method and compositions described herein. Such equivalents are intended to be encompassed by the following claims.

We claim:

1. A polypeptide comprising
(a) an amino acid sequence from the cytosolic domain of a CTLA-4, comprising between 25 and 41 contiguous amino acids of SEQ ID NO:3, or a functional fragment or variant thereof; and
(b) a heterologous amino acid sequence that is heterologous to CTLA-4.

2. A polypeptide comprising
(a) an amino acid sequence comprising at least 70% and less than 100% sequence identity to SEQ ID NO:3, or a homologue thereof, or functional fragment of the foregoing; and
(b) optionally a heterologous amino acid sequence that is heterologous to CTLA-4.

3. The polypeptide of claim 1 or 2, further comprising the amino acid sequence of SEQ ID NO:5, or a homologue thereof, or a functional fragment or variant thereof.

4. The polypeptide of claim 1 or 3, comprising the amino acid sequence of SEQ ID NO:7 or a homologue thereof, or a functional fragment or variant thereof.

5. The polypeptide of claim 3, further comprising one or more additional copies of the amino acid sequence of SEQ ID NO:5, or a homologue thereof, or a functional fragment or variant of the foregoing.

6. The polypeptide of any one of claims 1 or 3-5, comprising the amino acid sequence of SEQ ID NO:51, or a functional variant thereof.

7. The polypeptide of claim 1 or 3, wherein the amino acid sequence from the cytosolic domain of CTLA-4 consists of SEQ ID NO:3, SEQ ID NO:9, or SEQ ID NO:10, or a functional variant thereof having an amino acid sequence at least 70%, 75%, 80%, 85%, 90%, 95%, or 99% identical to SEQ ID NO: 3, SEQ ID NO:9, or SEQ ID NO:10.

8. The polypeptide of claim 7, wherein the polypeptide further comprises any one of SEQ ID NOs:3, 5, or 9-50, or 108-123, or a functional variant thereof having an amino acid sequence at least 70%, 75%, 80%, 85%, 90%, 95%, or 99% identical to any one of SEQ ID NOs:3, 5, or 9-50, or 108-123.

9. The polypeptide of any one of claims 1-8, wherein the polypeptide can interact with clathrin adaptor activating protein 2 (AP-2), optionally wherein interaction comprises the ability to co-immunoprecipitate.

10. The polypeptide of any one of claims 1-9, comprising two YVKM motif(s).

11. The polypeptide of any one of claims 1-10, wherein the heterologous sequence comprises one or more of a chimeric antigen receptor (CAR), programmed death protein 1 (PD1), protein transduction domain, fusogenic polypeptide, targeting signal, expression and/or purification tag.

12. The polypeptide of claim 11, wherein the heterologous sequence comprises a chimeric antigen receptor (CAR), and wherein the polypeptide is present within the intracellular region of the CAR.

13. The polypeptide of claim 11 or 12, wherein the heterologous sequence comprises a chimeric antigen receptor (CAR), and wherein the polypeptide is contiguous with the carboxyl terminus of the CAR.

14. The polypeptide of claim 11, or 12, or 13 wherein the heterologous sequence comprises a chimeric antigen receptor (CAR) comprising an intracellular component of CD3 zeta, and wherein the polypeptide is contiguous with the intracellular component of CD3 zeta.

15. The polypeptide of any one of claims 11-14, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

16. The polypeptide of claim 15, wherein the CAR targets one or more antigens selected from the group consisting of AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, XAGE, Claudin-6, Claudin-18.2 and CD70.

17. The polypeptide of claim 15, wherein the antigen is a cancer antigen selected from the group consisting of 4 1BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin.

18. The polypeptide of claim 17, wherein the CAR is anti CD19 or anti CD22, or both, optionally wherein the CAR is CD19BBz-CAR or CD22(m971)-CAR.

19. The polypeptide of any one of claims 1-6, wherein the heterologous sequence comprises the amino acid sequence of PD1, optionally wherein the polypeptide comprises the amino acid sequence of any one of SEQ ID NOS: 74, 76, or 126.

20. A nucleic acid comprising a nucleic acid encoding the polypeptide of any one of claims 1-19.

21. A nucleic acid comprising a nucleic acid encoding a polypeptide comprising a chimeric antigen receptor (CAR) and one or more of SEQ ID NO:3, or SEQ ID NO: 7, or SEQ ID NO:51.

22. The nucleic acid of any one of claims 20-21, wherein the nucleic acid is RNA or DNA.

23. The nucleic acid of any one of claims 20-22, wherein the nucleic acid is mRNA.

24. The nucleic acid of any one of claims 20-23, wherein the nucleic acid comprises an expression control sequence(s).

25. The nucleic acid of any one of claims 20-24, wherein the nucleic acid is, or is encoded by a vector or a transposon.

26. The nucleic acid of claim 25, wherein the vector is a viral vector.

27. The nucleic acid of claim 26, wherein the viral vector is selected from the group consisting of a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of a Adeno-associated virus (AAV) vector, Herpes Simplex virus (HSV) vector, vesicular stomatitis (VSV) vector, or a human Bocavirus vector (hBoV).

28. The nucleic acid of claim 25, wherein the vector is a nucleic acid expression vector selected from the group consisting of a plasmid, a cosmid, and a replicon.

29. The nucleic acid of any one of claims 20-28, wherein the nucleic acid comprises a promotor.

30. The nucleic acid of any one of claims 20-29, comprising one or more of a protein transduction domain, fusogenic polypeptide, or targeting signal conjugated thereto.

31. An isolated cell comprising the polypeptide of any one of claims 1-19, or the nucleic acid of any one of claims 20-30.

32. The isolated cell of claim 31, wherein the cell is a T cell, hematopoietic stem cell (HSC), macrophage, natural killer cell (NK), or dendritic cell (DC).

33. The isolated cell of claim 32, wherein the T cell is a CD8+ T cell selected from the group consisting of effector T cells, memory T cells, central memory T cells, and effector memory T cells.

34. The isolated cell of claim 32, wherein the T cell is a CD4+ T cell selected from the group consisting of Th1 cells, Th2 cells, Th17 cells, and Treg cells.

35. The isolated cell of any one of claims 31-34, wherein the polypeptide comprises
(i) an amino acid sequence encoding a CAR; and
(ii) an amino acid sequence of one or more of SEQ ID NO:3, or SEQ ID NO: 7, or SEQ ID NO:51, or a variant having at least 75% identity to SEQ ID NO:3, or at least 75% identity to SEQ ID NO: 7, or at least 75% identity to SEQ ID NO:51.

36. The isolated cell of any one of claims 31-35, wherein the polypeptide comprises
(i) an amino acid sequence encoding a CAR; and
(ii) an amino acid sequence of SEQ ID NO: 7, or a variant having at least 75% identity to SEQ ID NO: 7.

37. The isolated cell of claim 36, wherein the amino acid sequence of SEQ ID NO: 7 is contiguous with the residue at the carboxyl terminus of the CAR.

38. A population of cells derived by expanding the cell of any one of claims 31-37.

39. A pharmaceutical composition comprising the population of cells of claim 38 and a pharmaceutically acceptable buffer, carrier, diluent or excipient.

40. A method of treating a subject having a disease, disorder, or condition comprising administering to the subject an effective amount of the pharmaceutical composition of claim 39.

41. A method of treating a subject having a disease, disorder, or condition associated with an elevated expression or specific expression of an antigen, the method comprising administering to the subject an effective amount of a cell of claim 35 or 36, wherein the CAR targets the antigen, optionally wherein the cell is T cell, optionally a CD8+ T cell.

42. The method of any one of claims 40-41, wherein the cell was isolated from the subject having the disease, disorder, or condition prior to the introduction to the cell.

43. The method of any one of claims 40-41, wherein the cell was isolated from a healthy donor.

44. The method of any one of claims 40-43, wherein the subject is a human.

45. The method of any one of claims 40-44, wherein the subject has a disease selected from the group consisting of cancer, an inflammatory disease, a neuronal disorder, HIV/AIDS, a diabetes, a cardiovascular disease, an infectious disease (including a viral, a protozoan, a bacterial disease, and an allergy), an autoimmune disease and an autoimmune disease, and a genetic disorder.

46. The method of claim 45, wherein the disease is a cancer.

47. A method of introducing a CT fusion peptide into a cell, the method comprising introducing to the cell:
(i) a vector or transposon or mRNA encoding a polypeptide of any one of claims 1-19; and
(ii) causing the polypeptide to be expressed in the cell.

48. A chimeric antigen receptor (CAR) including a CT polypeptide (CAR-CT), wherein the CAR-CT targets CD22 and comprises the amino acid sequence of any one of SEQ ID NOs:55, 57, 59.

49. A chimeric antigen receptor (CAR) including a CT polypeptide (CAR-CT), wherein the CAR-CT targets CD22 and comprises the amino acid sequence of SEQ ID NO:57.

50. A chimeric antigen receptor (CAR) including a CT polypeptide (CAR-CT), wherein the CAR-CT targets CD19 and comprises the amino acid sequence of any one of SEQ ID NOs: 61 63, 65.

51. A chimeric antigen receptor (CAR) including a CT polypeptide (CAR-CT), wherein the CAR-CT targets CD19 and comprises the amino acid sequence of SEQ ID NO:63.

52. A nucleic acid, comprising a nucleic acid sequence encoding the chimeric antigen receptor of any one of claims 48-51.

53. The nucleic acid of claim 52, wherein the nucleic acid is a vector or a transposon.

54. An isolated cell comprising the CAR of any one of claims 48-51, or the nucleic acid of any one of claims 52-53.

55. A population of cells derived by expanding the cell of claim 54.

56. A pharmaceutical composition comprising the population of cells of claim 55 and a pharmaceutically acceptable buffer, carrier, diluent or excipient.

57. A method of treating a subject having a disease, disorder, or condition comprising administering to the subject an effective amount of the pharmaceutical composition of claim 56.

58. The method of claim 57, wherein the disease is a cancer.

59. The method of claim 46 or 58, wherein the cancer is a leukemia.


SEQUENCE LISTING (126 sequences)

SEQ ID NO: 1 - protein, 223 aa, Homo sapiens [sequence omitted]

SEQ ID NO: 2 - DNA, 672 bp, synthetic construct
  source 1..672: mol_type = other DNA
atggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 3 - protein, 41 aa, synthetic construct
AVSLSKMLKKRSPLTTGVYVKMPPTEPECEKQFQPYFIPIN

SEQ ID NO: 4 - DNA, 123 bp, synthetic construct
  source 1..123: mol_type = other DNA
gctgtttctttgagcaaaatgctaaagaaaagaagccctcttacaacaggggtctatgtgaaaatgcccccaacagagccagaatgtgaaaagcaatttcagccttattttattcccatcaat

SEQ ID NO: 5 - protein, 25 aa, synthetic construct
GVYVKMPPTEPECEKQFQPYFIPIN

SEQ ID NO: 6 - DNA, 75 bp, synthetic construct
  source 1..75: mol_type = other DNA
ggcgtctacgttaagatgccacccacggagccagaatgcgaaaaacagtttcaaccatatttcataccaataaac

SEQ ID NO: 7 - protein, 66 aa, synthetic construct
AVSLSKMLKKRSPLTTGVYVKMPPTEPECEKQFQPYFIPINGVYVKMPPTEPECEKQFQPYFIPIN

SEQ ID NO: 8 - DNA, 198 bp, synthetic construct
  source 1..198: mol_type = other DNA
gctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaac

SEQ ID NO: 9 - protein, 40 aa, synthetic construct
VSLSKMLKKRSPLTTGVYVKMPPTEPECEKQFQPYFIPIN

SEQ ID NO: 10 - protein, 39 aa, synthetic construct
SLSKMLKKRSPLTTGVYVKMPPTEPECEKQFQPYFIPIN

SEQ ID NO: 11 - protein, 38 aa, synthetic construct
LSKMLKKRSPLTTGVYVKMPPTEPECEKQFQPYFIPIN

SEQ ID NO: 12 - protein, 37 aa, synthetic construct
SKMLKKRSPLTTGVYVKMPPTEPECEKQFQPYFIPIN

SEQ ID NO: 13 - protein, 36 aa, synthetic construct
KMLKKRSPLTTGVYVKMPPTEPECEKQFQPYFIPIN

SEQ ID NO: 14 - protein, 35 aa, synthetic construct
MLKKRSPLTTGVYVKMPPTEPECEKQFQPYFIPIN

SEQ ID NO: 15 - protein, 34 aa, synthetic construct
LKKRSPLTTGVYVKMPPTEPECEKQFQPYFIPIN

SEQ ID NO: 16 - protein, 33 aa, synthetic construct
KKRSPLTTGVYVKMPPTEPECEKQFQPYFIPIN

SEQ ID NO: 17 - protein, 32 aa, synthetic construct
KRSPLTTGVYVKMPPTEPECEKQFQPYFIPIN

SEQ ID NO: 18 - protein, 31 aa, synthetic construct
RSPLTTGVYVKMPPTEPECEKQFQPYFIPIN

SEQ ID NO: 19 - protein, 30 aa, synthetic construct
SPLTTGVYVKMPPTEPECEKQFQPYFIPIN

SEQ ID NO: 20 - protein, 29 aa, synthetic construct
PLTTGVYVKMPPTEPECEKQFQPYFIPIN

SEQ ID NO: 21 - protein, 28 aa, synthetic construct
LTTGVYVKMPPTEPECEKQFQPYFIPIN

SEQ ID NO: 22 - protein, 27 aa, synthetic construct
TTGVYVKMPPTEPECEKQFQPYFIPIN

SEQ ID NO: 23 - protein, 26 aa, synthetic construct
TGVYVKMPPTEPECEKQFQPYFIPIN

SEQ ID NO: 24 - protein, 8 aa, synthetic construct
GVYVKMPP

SEQ ID NO: 25 - protein, 24 aa, synthetic construct
VYVKMPPTEPECEKQFQPYFIPIN

SEQ ID NO: 26 - protein, 23 aa, synthetic construct
YVKMPPTEPECEKQFQPYFIPIN

SEQ ID NO: 27 - protein, 39 aa, synthetic construct
VSLSKMLKKRSPLTTGVYVKMPPTEPECEKQFQPYFIPI

SEQ ID NO: 28 - protein, 38 aa, synthetic construct
VSLSKMLKKRSPLTTGVYVKMPPTEPECEKQFQPYFIP

SEQ ID NO: 29 - protein, 37 aa, synthetic construct
VSLSKMLKKRSPLTTGVYVKMPPTEPECEKQFQPYFI

SEQ ID NO: 30 - protein, 36 aa, synthetic construct
VSLSKMLKKRSPLTTGVYVKMPPTEPECEKQFQPYF

SEQ ID NO: 31 - protein, 35 aa, synthetic construct
VSLSKMLKKRSPLTTGVYVKMPPTEPECEKQFQPY

SEQ ID NO: 32 - protein, 34 aa, synthetic construct
VSLSKMLKKRSPLTTGVYVKMPPTEPECEKQFQP

SEQ ID NO: 33 - protein, 33 aa, synthetic construct
VSLSKMLKKRSPLTTGVYVKMPPTEPECEKQFQ

SEQ ID NO: 34 - protein, 32 aa, synthetic construct
VSLSKMLKKRSPLTTGVYVKMPPTEPECEKQF

SEQ ID NO: 35 - protein, 31 aa, synthetic construct
VSLSKMLKKRSPLTTGVYVKMPPTEPECEKQ

SEQ ID NO: 36 - protein, 30 aa, synthetic construct
VSLSKMLKKRSPLTTGVYVKMPPTEPECEK

SEQ ID NO: 37 - protein, 29 aa, synthetic construct
VSLSKMLKKRSPLTTGVYVKMPPTEPECE

SEQ ID NO: 38 - protein, 28 aa, synthetic construct
VSLSKMLKKRSPLTTGVYVKMPPTEPEC

SEQ ID NO: 39 - protein, 27 aa, synthetic construct
VSLSKMLKKRSPLTTGVYVKMPPTEPE

SEQ ID NO: 40 - protein, 26 aa, synthetic construct
VSLSKMLKKRSPLTTGVYVKMPPTEP

SEQ ID NO: 41 - protein, 25 aa, synthetic construct
VSLSKMLKKRSPLTTGVYVKMPPTE

SEQ ID NO: 42 - protein, 24 aa, synthetic construct
VSLSKMLKKRSPLTTGVYVKMPPT

SEQ ID NO: 43 - protein, 23 aa, synthetic construct
VSLSKMLKKRSPLTTGVYVKMPP

SEQ ID NO: 44 - protein, 22 aa, synthetic construct
VSLSKMLKKRSPLTTGVYVKMP

SEQ ID NO: 45 - protein, 38 aa, synthetic construct
SLSKMLKKRSPLTTGVYVKMPPTEPECEKQFQPYFIPI

SEQ ID NO: 46 - protein, 37 aa, synthetic construct
LSKMLKKRSPLTTGVYVKMPPTEPECEKQFQPYFIPI

SEQ ID NO: 47 - protein, 35 aa, synthetic construct
SKMLKKRSPLTTGVYVKMPPTEPECEKQFQPYFIP

SEQ ID NO: 48 - protein, 33 aa, synthetic construct
KMLKKRSPLTTGVYVKMPPTEPECEKQFQPYFI

SEQ ID NO: 49 - protein, 31 aa, synthetic construct
MLKKRSPLTTGVYVKMPPTEPECEKQFQPYF

SEQ ID NO: 50 - protein, 29 aa, synthetic construct
LKKRSPLTTGVYVKMPPTEPECEKQFQPY

SEQ ID NO: 51 - protein, 91 aa, synthetic construct
AVSLSKMLKKRSPLTTGVYVKMPPTEPECEKQFQPYFIPINGVYVKMPPTEPECEKQFQPYFIPINGVYVKMPPTEPECEKQFQPYFIPIN

SEQ ID NO: 52 - DNA, 273 bp, synthetic construct
  source 1..273: mol_type = other DNA
gctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaacggggtctatgttaaaatgccgcctactgagcctgagtgtgaaaaacaattccagccatattttatacccataaat

SEQ ID NO: 53 - protein, 116 aa, synthetic construct
AVSLSKMLKKRSPLTTGVYVKMPPTEPECEKQFQPYFIPINGVYVKMPPTEPECEKQFQPYFIPINGVYVKMPPTEPECEKQFQPYFIPINGVYVKMPPTEPECEKQFQPYFIPIN

SEQ ID NO: 54 - DNA, 348 bp, synthetic construct
  source 1..348: mol_type = other DNA
gctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaacggggtctatgttaaaatgccgcctactgagcctgagtgtgaaaaacaattccagccatattttatacccataaatggcgtctacgttaagatgccacccacggagccagaatgcgaaaaacagtttcaaccatatttcataccaataaac

SEQ ID NO: 55 - protein, 505 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAGTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAVSLSKMLKKRSPLTTGVYVKMPPTEPECEKQFQPYFIPIN

SEQ ID NO: 56 - DNA, 1515 bp, synthetic construct
  source 1..1515: mol_type = other DNA
caggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaggtaccaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctgtttctttgagcaaaatgctaaagaaaagaagccctcttacaacaggggtctatgtgaaaatgcccccaacagagccagaatgtgaaaagcaatttcagccttattttattcccatcaat

SEQ ID NO: 57 - protein, 530 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAGTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAVSLSKMLKKRSPLTTGVYVKMPPTEPECEKQFQPYFIPINGVYVKMPPTEPECEKQFQPYFIPIN

SEQ ID NO: 58 - DNA, 1590 bp, synthetic construct
  source 1..1590: mol_type = other DNA
caggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaggtaccaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaac

SEQ ID NO: 59 - protein, 575 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAGTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAVSLSKMLKKRSPLTTGVYVKMPPTEPECEKQFQPYFIPINGVYVKMPPTEPECEKQFQPYFIPINGVYVKMPPTEPECEKQFQPYFIPINCTEGRGSLLTCGDVEENPGP

SEQ ID NO: 60 - DNA, 1725 bp, synthetic construct
  source 1..1725: mol_type = other DNA
caggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaggtaccaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaacggggtctatgttaaaatgccgcctactgagcctgagtgtgaaaaacaattccagccatattttatacccataaattgtacagagggcagaggaagtctgctaacatgcggtgacgtcgaggagaatcctggccca

SEQ ID NO: 61 - protein, 506 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAVSLSKMLKKRSPLTTGVYVKMPPTEPECEKQFQPYFIPIN

SEQ ID NO: 62 - DNA, 1518 bp, synthetic construct
  source 1..1518: mol_type = other DNA
gacatccagatgacacagactacatcctccctgtctgcctctctgggagacagagtcaccatcagttgcagggcaagtcaggacattagtaagtatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtcacctcaaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctgtttctttgagcaaaatgctaaagaaaagaagccctcttacaacaggggtctatgtgaaaatgcccccaacagagccagaatgtgaaaagcaatttcagccttattttattcccatcaat

SEQ ID NO: 63 - protein, 531 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAVSLSKMLKKRSPLTTGVYVKMPPTEPECEKQFQPYFIPINGVYVKMPPTEPECEKQFQPYFIPIN

SEQ ID NO: 64 - DNA, 1593 bp, synthetic construct
  source 1..1593: mol_type = other DNA
gacatccagatgacacagactacatcctccctgtctgcctctctgggagacagagtcaccatcagttgcagggcaagtcaggacattagtaagtatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtcacctcaaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaac

SEQ ID NO: 65 - protein, 556 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAVSLSKMLKKRSPLTTGVYVKMPPTEPECEKQFQPYFIPINGVYVKMPPTEPECEKQFQPYFIPINGVYVKMPPTEPECEKQFQPYFIPIN

SEQ ID NO: 66 - DNA, 1668 bp, synthetic construct
  source 1..1668: mol_type = other DNA
gacatccagatgacacagactacatcctccctgtctgcctctctgggagacagagtcaccatcagttgcagggcaagtcaggacattagtaagtatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtcacctcaaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaacggggtctatgttaaaatgccgcctactgagcctgagtgtgaaaaacaattccagccatattttatacccataaat

SEQ ID NO: 67 - protein, 23 aa, synthetic construct
GSGSGEGRGSLLTCGDVEENPGP

SEQ ID NO: 68 - protein, 64 aa, synthetic construct
AVSLSKMLKKRSPLTTGVYVKMPPTEPECEKQFQPYFIPINGSGSGEGRGSLLTCGDVEENPGP

SEQ ID NO: 69 - DNA, 192 bp, synthetic construct
  source 1..192: mol_type = other DNA
gctgtttctttgagcaaaatgctaaagaaaagaagccctcttacaacaggggtctatgtgaaaatgcccccaacagagccagaatgtgaaaagcaatttcagccttattttattcccatcaatggatccggcagtggagagggcagaggaagtctgctaacatgcggtgacgtcgaggagaatcctggccca

SEQ ID NO: 70 - protein, 89 aa, synthetic construct
AVSLSKMLKKRSPLTTGVYVKMPPTEPECEKQFQPYFIPINGVYVKMPPTEPECEKQFQPYFIPINGSGSGEGRGSLLTCGDVEENPGP

SEQ ID NO: 71 - DNA, 267 bp, synthetic construct
  source 1..267: mol_type = other DNA
attgatgggaataaaataaggctgaaattgcttttcacattctggctctgttgggggcattttcacatagacccctgttgtaagagggcttcttttctttagcattttgctcaaagaaacagcggcgtctacgttaagatgccacccacggagccagaatgcgaaaaacagtttcaaccatatttcataccaataaacggatccggcagtggagagggcagaggaagtctgctaacatgcggtgacgtcgaggagaatcctggccca

SEQ ID NO: 72 - protein, 114 aa, synthetic construct
AVSLSKMLKKRSPLTTGVYVKMPPTEPECEKQFQPYFIPINGVYVKMPPTEPECEKQFQPYFIPINGVYVKMPPTEPECEKQFQPYFIPINGSGSGEGRGSLLTCGDVEENPGP

SEQ ID NO: 73 - DNA, 342 bp, synthetic construct
  source 1..342: mol_type = other DNA
gctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaacggggtctatgttaaaatgccgcctactgagcctgagtgtgaaaaacaattccagccatattttatacccataaatggatccggcagtggagagggcagaggaagtctgctaacatgcggtgacgtcgaggagaatcctggccca

SEQ ID NO: 74 - protein, 209 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIAVSLSKMLKKRSPLTTGVYVKMPPTEPECEKQFQPYFIPIN

SEQ ID NO: 75 - DNA, 627 bp, synthetic construct
  source 1..627: mol_type = other DNA
ttcttagactccccagacaggccctggaacccccccaccttctccccagccctgctcgtggtgaccgaaggggacaacgccaccttcacctgcagcttctccaacacatcggagagcttcgtgctcaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacctaggccagccggccagttccaaaccctggtggttggtgtcgtgggcggcctgctgggcagcctggtgctgctagtctgggtcctggccgtcatcgctgtttctttgagcaaaatgctaaagaaaagaagccctcttacaacaggggtctatgtgaaaatgcccccaacagagccagaatgtgaaaagcaatttcagccttattttattcccatcaat

SEQ ID NO: 76 - protein, 234 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIAVSLSKMLKKRSPLTTGVYVKMPPTEPECEKQFQPYFIPINGVYVKMPPTEPECEKQFQPYFIPIN

SEQ ID NO: 77 - DNA, 702 bp, synthetic construct
  source 1..702: mol_type = other DNA
ttcttagactccccagacaggccctggaacccccccaccttctccccagccctgctcgtggtgaccgaaggggacaacgccaccttcacctgcagcttctccaacacatcggagagcttcgtgctcaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacctaggccagccggccagttccaaaccctggtggttggtgtcgtgggcggcctgctgggcagcctggtgctgctagtctgggtcctggccgtcatcgctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaac

SEQ ID NO: 78 - DNA, 777 bp, synthetic construct
  source 1..777: mol_type = other DNA
ttcttagactccccagacaggccctggaacccccccaccttctccccagccctgctcgtggtgaccgaaggggacaacgccaccttcacctgcagcttctccaacacatcggagagcttcgtgctcaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacctaggccagccggccagttccaaaccctggtggttggtgtcgtgggcggcctgctgggcagcctggtgctgctagtctgggtcctggccgtcatcgctgtttctttgagcaaaatgctaaagaaaagaagccctcttacaacaggggtctatgtgaaaatgcccccaacagagccagaatgtgaaaagcaatttcagccttattttattcccatcaatggcgtctacgttaagatgccacccacggagccagaatgcgaaaaacagtttcaaccatatttcataccaataaacggggtctatgttaaaatgccgcctactgagcctgagtgtgaaaaacaattccagccatattttatacccataaat

SEQ ID NO: 79 - protein, 7 aa, synthetic construct
GQSSRSS

SEQ ID NO: 80 - protein, 6 aa, synthetic construct
GSGSGS

SEQ ID NO: 81 - protein, 6 aa, synthetic construct
ASGGGS

SEQ ID NO: 82 - protein, 10 aa, synthetic construct
GGGGSGGGGS

SEQ ID NO: 83 - protein, 20 aa, synthetic construct
GGGGSGGGGSGGGGSGGGGS

SEQ ID NO: 84 - protein, 8 aa, synthetic construct
GGGSGGGS

SEQ ID NO: 85 - protein, 15 aa, synthetic construct
GGGGSGGGGSGGGGS

SEQ ID NO: 86 - protein, 4 aa, synthetic construct
YVKM

SEQ ID NO: 87 - protein, 9 aa, synthetic construct
GDVEXNPGP

SEQ ID NO: 88 - protein, 82 aa, synthetic construct
AVSLSKMLKKRSPLTTGVYVKMPPTEPECEKQFQPYFIPINAVSLSKMLKKRSPLTTGVYVKMPPTEPECEKQFQPYFIPIN

SEQ ID NO: 89 - protein, 41 aa, Mus musculus; identical to SEQ ID NO: 3

SEQ ID NO: 90 - protein, 41 aa, Canis lupus
AVSLSKMLKKRSPLTTGVYVKMPPTGPECEKQFQPYFIPIN

SEQ ID NO: 91 - protein, 41 aa, Sus scrofa; identical to SEQ ID NO: 3

SEQ ID NO: 92 - protein, 41 aa, Rattus norvegicus
AVSLNRTLKKRSPLTTGVYVKMPPTEPECEKQFQPYFIPIN

SEQ ID NO: 93 - protein, 41 aa, Bos taurus; identical to SEQ ID NO: 3

SEQ ID NO: 94 - protein, 41 aa, Oryctolagus cuniculus; identical to SEQ ID NO: 3

SEQ ID NO: 95 - protein, 41 aa, Canis lupus; identical to SEQ ID NO: 3

SEQ ID NO: 96 - protein, 41 aa, Rattus norvegicus; identical to SEQ ID NO: 92

SEQ ID NO: 97 - protein, 41 aa, Ovis aries; identical to SEQ ID NO: 3

SEQ ID NO: 98 - protein, 41 aa, Macaca mulatta; identical to SEQ ID NO: 3

SEQ ID NO: 99 - protein, 41 aa, Ornithorhynchus anatinus
MVALSKMIKKRSLLTTGVYVKMPPPEPEHEKQFQPYFIPIN

SEQ ID NO: 100 - skipped

SEQ ID NO: 101 - protein, 41 aa, Equus caballus
AVSLSRMLKKRSPLTTGVYVKMPPTEPECEKQFQPYFIPIN

SEQ ID NO: 102 - protein, 41 aa, Papio anubis; identical to SEQ ID NO: 3

SEQ ID NO: 103 - protein, 41 aa, Nomascus leucogenys; identical to SEQ ID NO: 3

SEQ ID NO: 104 - protein, 41 aa, Myotis lucifugus; identical to SEQ ID NO: 3

SEQ ID NO: 105 - protein, 41 aa, Mustela putorius
AISLSKMLKKRSPLTTGVYVKMPPTEPECEKQFQPYFIPIN

SEQ ID NO: 106 - protein, 41 aa, Macaca nemestrina; identical to SEQ ID NO: 3

SEQ ID NO: 107 - protein, 41 aa, Cercocebus atys; identical to SEQ ID NO: 3

SEQ ID NO: 108 - protein, 24 aa, synthetic construct
GVYVKMPPTEPECEKQFQPYFIPI

SEQ ID NO: 109 - protein, 23 aa, synthetic construct
GVYVKMPPTEPECEKQFQPYFIP

SEQ ID NO: 110 - protein, 22 aa, synthetic construct
GVYVKMPPTEPECEKQFQPYFI

SEQ ID NO: 111 - protein, 21 aa, synthetic construct
GVYVKMPPTEPECEKQFQPYF

SEQ ID NO: 112 - protein, 20 aa, synthetic construct
GVYVKMPPTEPECEKQFQPY

SEQ ID NO: 113 - protein, 19 aa, synthetic construct
GVYVKMPPTEPECEKQFQP

SEQ ID NO: 114 - protein, 18 aa, synthetic construct
GVYVKMPPTEPECEKQFQ

SEQ ID NO: 115 - protein, 17 aa, synthetic construct
GVYVKMPPTEPECEKQF

SEQ ID NO: 116 - protein, 16 aa, synthetic construct
GVYVKMPPTEPECEKQ

SEQ ID NO: 117 - protein, 15 aa, synthetic construct
GVYVKMPPTEPECEK

SEQ ID NO: 118 - protein, 14 aa, synthetic construct
GVYVKMPPTEPECE

SEQ ID NO: 119 - protein, 13 aa, synthetic construct
GVYVKMPPTEPEC

SEQ ID NO: 120 - protein, 12 aa, synthetic construct
GVYVKMPPTEPE

SEQ ID NO: 121 - protein, 11 aa, synthetic construct
GVYVKMPPTEP

SEQ ID NO: 122 - protein, 10 aa, synthetic construct
GVYVKMPPTE

SEQ ID NO: 123 - protein, 9 aa, synthetic construct
GVYVKMPPT

SEQ ID NO: 124 - protein, 41 aa, synthetic construct; identical to SEQ ID NO: 3

SEQ ID NO: 125 - protein, 5 aa, synthetic construct
GGGGS

SEQ ID NO: 126 - protein, 259 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIAVSLSKMLKKRSPLTTGVYVKMPPTEPECEKQFQPYFIPINGVYVKMPPTEPECEKQFQPYFIPINGVYVKMPPTEPECEKQFQPYFIPIN